SEQUENCE LISTING (134 sequences)

SEQ ID NO: 1 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 2 - protein, 2531 aa, Mus musculus
MPRLLTPLLCLTLLPALAARGLRCSQPSGTCLNGGRCEVANGTEACVCSGAFVGQRCQDSNPCLSTPCKNAGTCHVVDHGGTVDYACSCPLGFSGPLCLTPLDNACLANPCRNGGTCDLLTLTEYKCRCPPGWSGKSCQQADPCASNPCANGGQCLPFESSYICRCPPGFHGPTCRQDVNECSQNPGLCRHGGTCHNEIGSYRCACRATHTGPHCELPYVPCSPSPCQNGGTCRPTGDTTHECACLPGFAGQNCEENVDDCPGNNCKNGGACVDGVNTYNCRCPPEWTGQYCTEDVDECQLMPNACQNGGTCHNTHGGYNCVCVNGWTGEDCSENIDDCASAACFQGATCHDRVASFYCECPHGRTGLLCHLNDACISNPCNEGSNCDTNPVNGKAICTCPSGYTGPACSQDVDECALGANPCEHAGKCLNTLGSFECQCLQGYTGPRCEIDVNECISNPCQNDATCLDQIGEFQCICMPGYEGVYCEINTDECASSPCLHNGHCMDKINEFQCQCPKGFNGHLCQYDVDECASTPCKNGAKCLDGPNTYTCVCTEGYTGTHCEVDIDECDPDPCHYGSCKDGVATFTCLCQPGYTGHHCETNINECHSQPCRHGGTCQDRDNSYLCLCLKGTTGPNCEINLDDCASNPCDSGTCLDKIDGYECACEPGYTGSMCNVNIDECAGSPCHNGGTCEDGIAGFTCRCPEGYHDPTCLSEVNECNSNPCIHGACRDGLNGYKCDCAPGWSGTNCDINNNECESNPCVNGGTCKDMTSGYVCTCREGFSGPNCQTNINECASNPCLNQGTCIDDVAGYKCNCPLPYTGATCEVVLAPCATSPCKNSGVCKESEDYESFSCVCPTGWQGQTCEVDINECVKSPCRHGASCQNTNGSYRCLCQAGYTGRNCESDIDDCRPNPCHNGGSCTDGINTAFCDCLPGFQGAFCEEDINECASNPCQNGANCTDCVDSYTCTCPVGFNGIHCENNTPDCTESSCFNGGTCVDGINSFTCLCPPGFTGSYCQYDVNECDSRPCLHGGTCQDSYGTYKCTCPQGYTGLNCQNLVRWCDSAPCKNGGRCWQTNTQYHCECRSGWTGVNCDVLSVSCEVAAQKRGIDVTLLCQHGGLCVDEGDKHYCHCQAGYTGSYCEDEVDECSPNPCQNGATCTDYLGGFSCKCVAGYHGSNCSEEINECLSQPCQNGGTCIDLTNSYKCSCPRGTQGVHCEINVDDCHPPLDPASRSPKCFNNGTCVDQVGGYTCTCPPGFVGERCEGDVNECLSNPCDPRGTQNCVQRVNDFHCECRAGHTGRRCESVINGCRGKPCKNGGVCAVASNTARGFICRCPAGFEGATCENDARTCGSLRCLNGGTCISGPRSPTCLCLGSFTGPECQFPASSPCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLSDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLAVEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACGSKEAKDLKARRKKSQDGKGCLLDSSSMLSPVDSLESPHGYLSDVASPPLLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHSMMGPLHSSLSTNTLSPIIYQGLPNTRLATQPHLVQTQQVQPQNLQLQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSPHSNISDWSEGISSPPTTMPSQITHIPEAFK

SEQ ID NO: 3 - protein, 1429 aa, Caenorhabditis elegans
MRIPTICFLFLLISLSKSLHIGSCLGLICGRNGHCHAGPVNGTQTSYWCRCDEGFGGEYCEQQCDVSKCGADEKCVFDKDYRMETCVCKDCDINGNSLLKPSCPSGYGGDDCKTQGWCYPSVCMNGGQCIGAGNRAKCACPDGFKGERCELDVNECEENKNACGNRSTCMNTLGTYICVCPQGFLPPDCLKPGNTSTVEFKQPVCFLEISADHPDGRSMYCQNGGFCDKASSKCQCPPGYHGSTCELLEKEDSCASNPCSHGVCISFSGGFQCICDDGYSGSYCQEGKDNCVNNKCEAGSKCINGVNSYFCDCPPERTGPYCEKMDCSAIPDICNHGTCIDSPLSEKAFECQCEPGYEGILCEQDKNECLSENMCLNNGTCVNLPGSFRCDCARGFGGKWCDEPLNMCQDFHCENDGTCMHTSDHSPVCQCKNGFIGKRCEKECPIGFGGVRCDLRLEIGICSRQGGKCFNGGKCLSGFCVCPPDFTGNQCEVNRKNGKSSLSENLCLSDPCMNNATCIDVDAHIGYACICKQGFEGDICERHKDLCLENPCSNGGVCHQHRESFSCDCPPGFYGNGCEQEKMFRCLKSTCQNGGVCINEEEKGRKCECSYGFSGARCEEKINLTGFTEKDSLLRSVCEKRKCSERANDGNCDADCNYAACKFDGGDCSGKREPFSKCRYGNMCADFFANGVCNQACNNEECLYDGMDCLPAVVRCPVKIREHCASRFANGICDPECNTNGCGFDGGDCDNETNATIITNIRITVQMDPKEFQVTGGQSLMEISSALRVTVRIQRDEEGPLVFQWNGESEMDRVKMNERQLTEQHVLSTSISRKIKRSATNIGVVVYLEVQENCDTGKCLYKDAQSVVDSISARLAKKGIDSFGIPISEALVAEPRKSGNNTGFLSWNALLLIGAGCLIVMVVLMLGALPGNRTRKRRMINASVWMPPMENEEKNRKNHQSITSSQHSLLEASYDGYIKRQRNELQHYSLYPNPQGYGNGNDFLGDFNHTNLQIPTEPEPESPIKLHTEAAGSYAITEPITRESVNIIDPRHNRTVLHWIASNSSAEKSEDLIVHEAKECIAAGADVNAMDCDENTPLMLAVLARRRRLVAYLMKAGADPTIYNKSERSALHQAAANRDFGMMVYMLNSTKLKGDIEELDRNGMTALMIVAHNEGRDQVASAKLLVEKGAKVDYDGAARKDSEKYKGRTALHYAAQVSNMPIVKYLVGEKGSNKDKQDEDGKTPIMLAAQEGRIEVVMYLIQQGASVEAVDATDHTARQLAQANNHHNIVDIFDRCRPEREYSMDLHIQHTHQPQPSRKVTRAPKKQTSRSKKESASNSRDSTHLTPPPSDGSTSTPSPQHFMNTTHTTPTSLNYLSPEYQTEAGSSEAFQPQCGAFGNGEMWYTRASTSYTQMQNEPMTRYSEPAHYF

SEQ ID NO: 4 - protein, 2703 aa, Drosophila melanogaster
MQSQRSRRRSRAPNTWICFWINKMHAVASLPASLPLLLLTLAFANLPNTVRGTDTALVAASCTSVGCQNGGTCVTQLNGKTYCACDSHYVGDYCEHRNPCNSMRCQNGGTCQVTFRNGRPGISCKCPLGFDESLCEIAVPNACDHVTCLNGGTCQLKTLEEYTCACANGYTGERCETKNLCASSPCRNGATCTALAGSSSFTCSCPPGFTGDTCSYDIEECQSNPCKYGGTCVNTHGSYQCMCPTGYTGKDCDTKYKPCSPSPCQNGGICRSNGLSYECKCPKGFEGKNCEQNYDDCLGHLCQNGGTCIDGISDYTCRCPPNFTGRFCQDDVDECAQRDHPVCQNGATCTNTHGSYSCICVNGWAGLDCSNNTDDCKQAACFYGATCIDGVGSFYCQCTKGKTGLLCHLDDACTSNPCHADAICDTSPINGSYACSCATGYKGVDCSEDIDECDQGSPCEHNGICVNTPGSYRCNCSQGFTGPRCETNINECESHPCQNEGSCLDDPGTFRCVCMPGFTGTQCEIDIDECQSNPCLNDGTCHDKINGFKCSCALGFTGARCQINIDDCQSQPCRNRGICHDSIAGYSCECPPGYTGTSCEININDCDSNPCHRGKCIDDVNSFKCLCDPGYTGYICQKQINECESNPCQFDGHCQDRVGSYYCQCQAGTSGKNCEVNVNECHSNPCNNGATCIDGINSYKCQCVPGFTGQHCEKNVDECISSPCANNGVCIDQVNGYKCECPRGFYDAHCLSDVDECASNPCVNEGRCEDGINEFICHCPPGYTGKRCELDIDECSSNPCQHGGTCYDKLNAFSCQCMPGYTGQKCETNIDDCVTNPCGNGGTCIDKVNGYKCVCKVPFTGRDCESKMDPCASNRCKNEAKCTPSSNFLDFSCTCKLGYTGRYCDEDIDECSLSSPCRNGASCLNVPGSYRCLCTKGYEGRDCAINTDDCASFPCQNGGTCLDGIGDYSCLCVDGFDGKHCETDINECLSQPCQNGATCSQYVNSYTCTCPLGFSGINCQTNDEDCTESSCLNGGSCIDGINGYNCSCLAGYSGANCQYKLNKCDSNPCLNGATCHEQNNEYTCHCPSGFTGKQCSEYVDWCGQSPCENGATCSQMKHQFSCKCSAGWTGKLCDVQTISCQDAADRKGLSLRQLCNNGTCKDYGNSHVCYCSQGYAGSYCQKEIDECQSQPCQNGGTCRDLIGAYECQCRQGFQGQNCELNIDDCAPNPCQNGGTCHDRVMNFSCSCPPGTMGIICEINKDDCKPGACHNNGSCIDRVGGFECVCQPGFVGARCEGDINECLSNPCSNAGTLDCVQLVNNYHCNCRPGHMGRHCEHKVDFCAQSPCQNGGNCNIRQSGHHCICNNGFYGKNCELSGQDCDSNPCRVGNCVVADEGFGYRCECPRGTLGEHCEIDTLDECSPNPCAQGAACEDLLGDYECLCPSKWKGKRCDIYDANYPGWNGGSGSGNDRYAADLEQQRAMCDKRGCTEKQGNGICDSDCNTYACNFDGNDCSLGINPWANCTANECWNKFKNGKCNEECNNAACHYDGHDCERKLKSCDSLFDAYCQKHYGDGFCDYGCNNAECSWDGLDCENKTQSPVLAEGAMSVVMLMNVEAFREIQAQFLRNMSHMLRTTVRLKKDALGHDIIINWKDNVRVPEIEDTDFARKNKILYTQQVHQTGIQIYLEIDNRKCTECFTHAVEAAEFLAATAAKHQLRNDFQIHSVRGIKNPGDEDNGEPPANVKYVITGIILVIIALAFFGMVLSTQRKRAHGVTWFPEGFRAPAAVMSRRRRDPHGQEMRNLNKQVAMQSQGVGQPGAHWSDDESDMPLPKRQRSDPVSGVGLGNNGGYASDHTMVSEYEEADQRVWSQAHLDVVDVRAIMTPPAHQDGGKHDVDARGPCGLTPLMIAAVRGGGLDTGEDIENNEDSTAQVISDLLAQGAELNATMDKTGETSLHLAARFARADAAKRLLDAGADANCQDNTGRTPLHAAVAADAMGVFQILLRNRATNLNARMHDGTTPLILAARLAIEGMVEDLITADADINAADNSGKTALHWAAAVNNTEAVNILLMHHANRDAQDDKDETPLFLAAREGSYEACKALLDNFANREITDHMDRLPRDVASERLHHDIVRLLDEHVPRSPQMLSMTPQAMIGSPPPGQQQPQLITQPTVISAGNGGNNGNGNASGKQSNQTAKQKAAKKAKLIEGSPDNGLDATGSLRRKASSKKTSAASKKAANLNGLNPGQLTGGVSGVPGVPPTNSAAQAAAAAAAAVAAMSHELEGSPVGVGMGGNLPSPYDTSSMYSNAMAAPLANGNPNTGAKQPPSYEDCIKNAQSMQSLQGNGLDMIKLDNYAYSMGSPFQQELLNGQGLGMNGNGQRNGVGPGVLPGGLCGMGGLSGAGNGNSHEQGLSPPYSNQSPPHSVQSSLALSPHAYLGSPSPAKSRPSLPTSPTHIQAMRHATQQKQFGGSNLNSLLGGANGGGVVGGGGGGGGGVGQGPQNSPVSLGIISPTGSDMGIMLAPPQSSKNSAIMQTISPQQQQQQQQQQQQQHQQQQQQQQQQQQQQQQQLGGLEFGSAGLDLNGFCGSPDSFHSGQMNPPSIQSSMSGSSPSTNMLSPSSQHNQQAFYQYLTPSSQHSGGHTPQHLVQTLDSYPTPSPESPGHWSSSSPRSNSDWSEGVQSPAANNLYISGGHQANKGSEAIYI

SEQ ID NO: 5 - protein, 2900 aa, Bos taurus
MTPVCTPTRPGPCAHPALPRSTPHSITDSSRAEPIESFLVLSPALELRLLLAVVGQDTPLGDVWAGGKASGGGDTEGPLSEGSKEGEAATGPQAPGAEWHAPPRSTCLSSTPRPEAVPPSLPCRSPGWGACGGRRPGPALEPAHMGSVPSQQRPPGLDRSREDNGPPQPLPSPHGGASLAPAPPPACRGWQPPLRWPGAAAARVPGHRRTCSPAALCPCCRCLIYWARFSSHCNSRSLPGQDALGPGLWVPSATQAGTRQPVTGSTLSGGHATFPRLQGMALPEPEGEGPPTTSAQGCGPSVRAAFPGRCQLGAVGAFHPRGSAAGKREAWLVPEPLLGFPSSSRLRGDPGGHVPPRLKKPRQQVAKGGPGAGVAGAEPFPVGSAGDQAWGWAGGKAPPTPGSPATVAAREPAQGLPDCGGAFAGQQCQAPNPCLSAPCKNGGTCHTTEREGLVDYVCGCRLGFSGPLCLTPRDHACLASPCLNGGTCDLLTLTEYKCLCTPGWSGKTCQQADPCASNPCANGGQCLPFEASYICHCPPGFHGPTCRQDVNECSQSPGLCHHGGTCLNEVGSYRCVCRPTHTGPHCELPYVPCSPSPCQNGGTCRPTGDTTHECACLPGFTGQNCEENIDDCPGNSCKNGGACVDGVNTYNCRCPPEWTGQYCTEDVDECQLMPNACQNGGTCHNTHGGYNCVCVNGWTGEDCSENIDDCASASCFQGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGLHCEVNTDECASSPCLQNGRCLDKINEFVCECPTGFTGHLCQYDVDECASTPCKNGAKCLDGPNTYTCVCTEGYTGPHCEVDIDECDPDPCHYGSCKDGVATFTCLCQPGYTGHHCESNINECHSQPCRHGGTCQDRDNAYLCFCLKGTTGPNCEINLDDCASNPCDSGTCLDKIDGYECACEPGYTGSMCNINIDECADSPCHNGGTCEDGINGFTCRCPEGYHDPTCLSEVNECSSNPCIHGACRDSLNGYKCDCDPGWSGANCDVNNDECESNPCINGGTCKDMTSGYVCACREGFSGPNCQTNINECASNPCLNQGTCIDDVAGYKCNCLLPYTGATCEVVLAPCAPGPCRNGGECRESEDYESFSCACPAGWQGQTCEIDINECVKSPCRAGASCQNTNGSYRCHCQAGYTGRNCETDIDDCRPNPCHNGGSCTDGINTAFCDCLPGFQGAFCEEDINECASSPCRNGANCTDCVDSYTCTCPTGFSGIHCENNTPDCTESSCFNGGTCVDGINSFTCLCPPGFTGSYCQHDVNECDSRPCLHGGTCHDSYGTYTCTCPQGYTGLNCQTLVRWCDSSPCKNDGRCWQTNALYRCECHSGWTGLYCDVPSVSCEVAARQQGVNVTHLCRNGGLCMNAGNTHRCHCQAGYTGSYCEEQVDECSPSPCQNGATCTDYPGGYSCECVAGYHGVNCSEEVNECLSQPCRNGGTCIDLTNTYKCSCPRGTQGVHCEINVDDCNPPIDPVSRGPKCFNNGTCVDQVGGYSCSCPPGFVGERCEGDVNECLSNPCDARGTQNCVQHVNAFHCECRAGHTGRRCESVINGCKDRPCKNGGSCAVASNTARGFICKCPAGFEGATCENDARSCGSLRCLNGGTCIAGPRSPTCLCLGPFTGPECQFPASSPCVGGNPCYNQGVCEPTAESPFYRCRCPAKFNGLLCHILDYSFGGGVGLDIPPPQIEETCELPGCREEAGNKVCSLQCNSHACGWDGGDCSLDFDDPWQNCTQSLQCWKYFSNGRCDSQCNSAGCLFDGFDCQRAEGQCNPLYDQYCKDHFRDGHCDQGCNSAECEWDGLDCAEHVPERLAAGTLVLVVLMPPEQLRNRSLHFLRELSRLLHTNVVFKRDASGQQMIFPYYGRAPLPAGERSEECRCEHHACPAGAGQGEPSGPLCTSRSIVYLEIDNRQCVQSSSQCFQSATDVAAFLGALASLGSLNIPYKIEAVQSETVEPPPPPPLHFMYVAVVAFVLLFFVGCGVLLSRKRRRQHGQLWFPEGFKVSEASKKKRREPLGEDSVGLKPLKNSSDGALMDDNQNEWGDEGLEAKKFRFEEPVVLPDLDDQTDHRQWTQQHLDAADLRVSAMAPTPPQGEADADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVEAAVVLLKNGANKDMQNNKEETPLFLAAREGSYETAKVLLDHFANRDITDHMDRLPRDIAQERMHHDIVRLLDEYSLVRSPPLHGATLGGTPTLSPPLCSPNGYLGNLKPPMQGKKARKPSTKGLACGGKEPKDLKARRKKSQDGKGCLLDSGSVMSPVDSLESPHGYLSDVASPPLLPSPFQPSPSVPLNHLPGMPETHLGVSHLSVAAKPEMAVLSGGSRLAFEAGPPRLSHLPVASSTSTILGSGGSGGSGAVNFTVGGAAGLNGQCEWLSRLQNGLVPNQYNPLRGGVTPGTLSTQAAGLQHGTVGPLHAPALSQVMTYQALPSTRLASQPHLVQPQQNLQMQPPSMPPQPNLQPHLGVSSAASGHLGRSFLGGELSQADMQPLGPGNLAAHTVLPQDGQVLPTSLPSTLAPPTMAPPMTTAQFLTPPSQHSYSSSPVDNTPSHQLQVPEHPFLTPSPESPDQWSSSSPHSNISDWSEGISSPPTSVPSQIAHVPEAFK

SEQ ID NO: 6 - protein, 2561 aa, Gallus gallus
MGRCSAAHPRGGVHCPGLCAVPDALLLFPGVRCTQLAESCLNGGKCETFLNGTEVCQCSSAHMGERCQLPNPCLSSPCKNAGTCIPLLRGSTADYTCVCRLGFTDELCLTPLDNACLNNPCRNGGTCDLVTLSEYKCRCPPGWSGKTCQQADPCASNPCANGGQCVPFEAHYICRCTAGFHGANCKQDVNECNISPPVCKNGGSCTNEVGTYQCSCKPAYTGQNCEHLYVPCNPSPCQNGGTCRQTGDTTYDCTCLPGFTGQNCEENIDDCPGNNCRNGGTCVDGVNTYNCQCPPEWTGQYCTEDVDECQLMPNACQNGGTCHNNHGGYNCVCVNGWTGEDCSENIDDCAMAACFQGATCHDRVASFYCECPHGRTGLLCHLDDACISNPCNEGSNCDTNPVNGKAICTCPSGYMGPACNQDVDECSLGANPCEHAGKCINTQGSFQCQCLQGYSGPRCEIDVNECLSNPCQNDATCLDQIGEFQCICMPGYEGVYCEINTDECASSPCLHNGNCLDKINEFHCECPTGFNGHLCQFDIDECASTPCKNGAKCVDGPNTYSCECTEGFSGVHCEIDIDECNPDPCHYGTCKDSIAAFTCLCQPGYTGHRCDININECQSQPCRNGGTCQDRDNAYNCLCLKGTTGPNCEINLDDCASNPCDYGKCIDKINGYECTCEPGYTGRMCNINIDECASNPCHNGGTCKDGINGFTCLCPEGFHDPKCLSEVNECNSNPCIHGRCHDGLNGYRCDCDPGWSGTNCDINNNECESNPCMNGGTCKDMTSGYICTCREGFSGPNCQTNINECASNPCLNQGTCIDDVAGYTCNCLLPYTGATCEDVLAPCAGGPCKNGGECRESEDYKRFSCSCPPGWQGQTCEIDINECVKSPCRNGATCQNTNGSYRCLCRVGFAGRNCDTDIDDCQPNPCHNGGSCSDGIGTFFCECLAGFRGLKCEEDINECASNPCKNGANCTDCVNSYTCTCPSGFSGIHCENNTPDCTESSCFNGGTCVDGINTFTCLCPSGFTGSYCEHNINECDSKPCLNGGTCQDSYGTYKCTCPQGYTGLNCQNLVRWCDSSPCKNGGKCWQTNNLYRCECNSGWTGLYCDVPSVSCEVAAKQQGIDVAHLCRNSGLCVDSGNTHFCRCQAGYTGSYCEEQVDECSPNPCQNGATCTDYLGGYSCECVAGYHGVNCSEEINECLSHPCQNGGTCIDLINTYKCSCPRGTQGVHCEINVDDCSPFFDPVTLGPKCFNNGKCTDRVGGYSCICPPGFVGERCEGDVNECLSNPCDARGTQNCVQRVNDYKCECRPGYAGRRCDTVVDGCKGKPCRNGGTCAVASNTGRGFICKCPPGFVGATCENDSHTCGTLHCLNGGTCISMHKSSKCVCAAAFTGPECQYPASSPCISNPCYNGGTCEFLSDASPYYHCNCPANFNGLNCHILDFDFQGGFGQDIIPPKIEEKCEIAVCASYAGNKICDGKCNNHACGWDGGDCSLNFNDPWKNCSQSLQCWKYFNDGKCDSQCNNAGCLYDGFDCQKYEGQCNPLYDQYCKDHFSDGHCDQGCNNFECEWDGLDCANNMPEKLADGTLVVVVLITPENLKNNSFNFLRELSRVLHTNVVFKKNAKGEYMIFPYYGNEEELKKHYIKRSTEDWADMSSAVINKVKSSLYSRAGRRQKRELDQMDIRGSIVYLEIDNRQCIQSSSQCFQSATDVAAFLGALASLGNLNIPYKIEAVKSETAEPARNSQLYPMYVVVAALVLLAFIGVGVLVSRKRRREHGQLWFPEGFKVTESSKKKRREPLGEDSVGLKPLKNASDGTLMDDNQNEWGDEETLDTKKFRFEEQAMLPDTDDQTDHRQWTQQHLDAADLRISSMAPTPPQGEIDADCMDVNVRGPDGFTPLMIASCSGGGLETGNSEEEDDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINCHADVNAVDDLGKSALHWAAAVNNVEAAVVLLKNGANKDMQNNKEETPLFLAAREGSYETAKVLLDHFANRDITDHMDRLPRDIAQERMHHDIVRLLDEYNLVRSPPLHSGPLGAPTLSPPLCSPSSYIGNLKPAVQGKKARKPSTKGLSCNGKDSKDLKARRKKSQDGKGCLLDNSSVLSPVDSLESPHGYLSDVASPPLMTSPFQQSPSMPLNHLPGMPDAHMSINHLNMAGKQEMALGGSGRMAFEAVPPRLSHLPVSSPSTAMSNAPMNFSVGGAAGLSGQCDWLSRLQSGMVQNQYGAMRGGMQPGTHQQAQNLQHGMMSSLHNGLPSTSLSQMMSYQAMPSTRLASQPHLLQNQQMQQMQQPGMQPQPGMQPQPGMQQPQQQPQQQPQPQQHHNPGSNASGHMGQNFLGTELSQPDMQPVSSSAMAVHTILPQDSQLLPTSLPSSLAQPMTTTQFLTPPSQHSYSSPLDNTPSHQLQVPDHPFLTPSPESPDQWSSSSPHSNVSDWSEGISSPPTSMQSQMGHIPEAFK

SEQ ID NO: 7 - protein, 2531 aa, Rattus rattus
MPRLLAPLLCLTLLPALAARGLRCSQPSGTCLNGGRCEVANGTEACVCSGAFVGQRCQDPSPCLSTPCKNAGTCYVVDHGGIVDYACSCPLGFSGPLCLTPLANACLANPCRNGGTCDLLTLTEYKCRCPPGWSGKSCQQADPCASNPCANGGQCLPFESSYICGCPPGFHGPTCRQDVNECSQNPGLCRHGGTCHNEIGSYRCACRATHTGPHCELPYVPCSPSPCQNGGTCRPTGDTTHECACLPGFAGQNCEENVDDCPGNNCKNGGACVDGVNTYNCRCPPEWTGQYCTEDVDECQLMPNACQNAGTCHNSHGGYNCVCVNGWTGEDCSDNIDDCASAACFQGATCHDRVASFYCECPHGRTGLLCHLNDACISNPCNEGSNCDTNPVNGKAICTCPRGYTGPACSQDVDECALGANPCEHAGKCLNTLGSFECQCLQGYTGPRCEIDVNECISNPCQNDATCLDQIGEFQCICMPGYEGVYCEINTDECASSPCLHNGRCVDKINEFLCQCPKGFSGHLCQYDVDECASTPCKNGAKCLDGPNTYTCVCTEGYTGTHCEVDIDECDPDPCHIGLCKDGVATFTCLCQPGYTGHHCETNINECHSQPCRHGGTCQDRDNYYLCLCLKGTTGPNCEINLDDCASNPCDSGTCLDKIDGYECACEPGYTGSMCNVNIDECAGSPCHNGGTCEDGIAGFTCRCPEGYHDPTCLSEVNECNSNPCIHGACRDGLNGYKCDCAPGWSGTNCDINNNECESNPCVNGGTCKDMTSGYVCTCREGFSGPNCQTNINECASNPCLNQGTCIDDVAGYKCNCPLPYTGATCEVVLAPCATSPCKNSGVCKESEDYESFSCVCPTGWQGQTCEIDINECVKSPCRHGASCQNTNGSYRCLCQAGYTGRNCESDIDDCRPNPCHNGGSCTDGVNAAFCDCLPGFQGAFCEEDINECATNPCQNGANCTDCVDSYTCTCPTGFNGIHCENNTPDCTESSCFNGGTCVDGINSFTCLCPPGFTGSYCQYDVNECDSRPCLHGGTCQDSYGTYKCTCPQGYTGLNCQNLVRWCDSAPCKNGGKCWQTNTQYHCECRSGWTGFNCDVLSVSCEVAAQKRGIDVTLLCQHGGLCVDEEDKHYCHCQAGYTGSYCEDEVDECSPNPCQNGATCTDYLGGFSCKCVAGYHGSNCSEEINECLSQPCQNGGTCIDLTNTYKCSCPRGTQGVHCEINVDDCHPPLDPASRSPKCFNNGTCVDQVGGYTCTCPPGFVGERCEGDVNECLSNPCDPRGTQNCVQRVNDFHCECRAGHTGRRCESVINGCRGKPCRNGGVCAVASNTARGFICRCPARFEGATCENDARTCGSLRCLNGGTCISGPRSPTCLCLGSFTGPECQFPASSPCVGSNPCYNQGTCEPTSESPFYRCLCPAKFNGLLCHILDYSFTGAAGRDIPPPQIEEACELPECQEDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRDVSHVLHTNVVFKRDAQGQQMIFPYYGREEELRKHPIKRSAVGWATTSLLPGTNGGRQRRELDPMDIHGSIVYLEIDNRQCVQSSSQCFQSATDVAAFLGALASLGSLNIPYKIEAVKSETVEPPLPSQLHLMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRVSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILLRNRATDLDARMHDGTTPLILAARLAVEGMLEDLINSHADVNAVDDLGKSALHWAAAVNNVDAAVVLLKNGANKDMQNNKEETPLFLAAREGSYETAKVLLDHFANRDITDHMDRLPRDIAQERMHHDIVRLLDEYNLVRSPQLHGTALGGTPTLSPTLCSPNGYLGNLKSATQGKKARKPSTKGLACSSKEAKDLKARRKKSQDGKGCLLDSSSMLSPVDSLESPHGYLSDVASPPLLPSPFQQSPSMPLSHLPGMPDTHLGISHLNVAAKPEMAALAGGSRLAFEPPPPRLSHLPVASSASTVLSTNGTGAMNFTVGAPASLNGQCEWLPRLQNGMVPSQYNPLRPGVTPGTLSTQAAGLQHGMMGPIHSSLSTNTLSPIIYQGLPNTRLATQPHLVQTQQVQPQNLQIQPQNLQPPSQPHLSVSSAANGHLGRSFLSGEPSQADVQPLGPSSLPVHTILPQESQALPTSLPSSMVPPMTTTQFLTPPSQHSYSSSPVDNTPSHQLQVPEHPFLTPSPESPDQWSSSSRHSNISDWSEGISSPPTSMPSQITHIPEAFK

SEQ ID NO: 8 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 9 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 10 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 11 - protein, 2470 aa, Mus musculus
MPDLRPAALRALLWLWLCGAGPAHALQCRGGQEPCVNEGTCVTYHNGTGFCRCPEGFLGEYCQHRDPCEKNRCQNGGTCVPQGMLGKATCRCAPGFTGEDCQYSTSHPCFVSRPCQNGGTCHMLSRDTYECTCQVGFTGKQCQWTDACLSHPCENGSTCTSVASQFSCKCPAGLTGQKCEADINECDIPGRCQHGGTCLNLPGSYRCQCGQGFTGQHCDSPYVRGLPCVNGGTCRQTGDFTLECNCLPGFEGSTCERNIDDCPNHKCQNGGVCVDGVNTYNCRCPPQWTGQFCTEDVDECLLQPNACQNGGTCTNRNGGYGCVCVNGWSGDDCSENIDDCAYASCTPGSTCIDRVASFSCLCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEVNECQSNPCVNNGQCVDKVNRFQCLCPPGFTGPVCQIDIDDCSSTPCLNGAKCIDHPNGYECQCATGFTGILCDENIDNCDPDPCHHGQCQDGIDSYTCICNPGYMGAICSDQIDECYSSPCLNDGRCIDLVNGYQCNCQPGTSGLNCEINFDDCASNPCMHGVCVDGINRYSCVCSPGFTGQRCNIDIDECASNPCRKGATCINDVNGFRCICPEGPHHPSCYSQVNECLSNPCIHGNCTGGLSGYKCLCDAGWVGVNCEVDKNECLSNPCQNGGTCNNLVNGYRCTCKKGFKGYNCQVNIDECASNPCLNQGTCFDDVSGYTCHCMLPYTGKNCQTVLAPCSPNPCENAAVCKEAPNFESFSCLCAPGWQGKRCTVDVDECISKPCMNNGVCHNTQGSYVCECPPGFSGMDCEEDINDCLANPCQNGGSCVDHVNTFSCQCHPGFIGDKCQTDMNECLSEPCKNGGTCSDYVNSYTCTCPAGFHGVHCENNIDECTESSCFNGGTCVDGINSFSCLCPVGFTGPFCLHDINECSSNPCLNAGTCVDGLGTYRCICPLGYTGKNCQTLVNLCSRSPCKNKGTCVQEKARPHCLCPPGWDGAYCDVLNVSCKAAALQKGVPVEHLCQHSGICINAGNTHHCQCPLGYTGSYCEEQLDECASNPCQHGATCNDFIGGYRCECVPGYQGVNCEYEVDECQNQPCQNGGTCIDLVNHFKCSCPPGTRGLLCEENIDECAGGPHCLNGGQCVDRIGGYTCRCLPGFAGERCEGDINECLSNPCSSEGSLDCVQLKNNYNCICRSAFTGRHCETFLDVCPQKPCLNGGTCAVASNMPDGFICRCPPGFSGARLQSSCGQVKCRRGEQCIHTDSGPRCFCLNPKDCESGCASNPCQHGGTCYPQRQPPHYSCRCPPSFGGSHCELYTAPTSTPPATCQSQYCADKARDGICDEACNSHACQWDGGDCSLTMEDPWANCTSTLRCWEYINNQCDEQCNTAECLFDNFECQRNSKTCKYDKYCADHFKDNHCDQGCNSEECGWDGLDCASDQPENLAEGTLIIVVLLPPEQLLQDSRSFLRALGTLLHTNLRIKQDSQGALMVYPYFGEKSAAMKKQKMTRRSLPEEQEQEQEVIGSKIFLEIDNRQCVQDSDQCFKNTDAAAALLASHAIQGTLSYPLVSVFSELESPRNAQLLYLLAVAVVIILFFILLGVIMAKRKQAWLPLAAGRFTLRRDSSNHKRREPVGQDAVGLKNLSVQVSEANLIGSGTSEHWVDDEGPQPKKAKAEDEALLSEDDPIDRRPWTQQHLEAADISHTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADRNAQDNMGRCPLHAAVAGDAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVLCGPNRSFLSLKHTPMGKKARRPNTKSTMPTSLPNLAKEAKDAKGSRRKKCLNEKVQLSESSVTLSPVDSLESPHTYVSDATSSPMITSPGILQASPTPLLAAAAPAAPVHTQHALSFSNLHDMQPLAPGASTVLPSVSQLLSHHHIAPPGSSSAGSLGRLHPVPVPADWMNRVEMNETQYSEMFGMVLAPAEGAHPGIAAPQSRPPEGKHMSTQREPLPPIVTFQLIPKRSIAQAAGAPQTQSSCPPAVAGPLPSMYQIPEMPRLPSVAFPPTMMPQQEGQVAQTIVPTYHPFPASVGKYPTPPSQHSYASSNAAERTPSHGGHLQGEHPYLTPSPESPDQWSSSSPHSASDWSDVTTSPTPGGGGGGQRGPGTHMSEPPHSNMQVYA

SEQ ID NO: 12 - protein, 1964 aa, Mus musculus
MQPQLLLLLLLPLNFPVILTRELLCGGSPEPCANGGTCLRLSQGQGICQCAPGFLGETCQFPDPCRDTQLCKNGGSCQALLPTPPSSRSPTSPLTPHFSCTCPSGFTGDRCQTHLEELCPPSFCSNGGHCYVQASGRPQCSCEPGWTGEQCQLRDFCSANPCANGGVCLATYPQIQCRCPPGFEGHTCERDINECFLEPGPCPQGTSCHNTLGSYQCLCPVGQEGPQCKLRKGACPPGSCLNGGTCQLVPEGHSTFHLCLCPPGFTGLDCEMNPDDCVRHQCQNGATCLDGLDTYTCLCPKTWKGWDCSEDIDECEARGPPRCRNGGTCQNTAGSFHCVCVSGWGGAGCEENLDDCAAATCAPGSTCIDRVGSFSCLCPPGRTGLLCHLEDMCLSQPCHVNAQCSTNPLTGSTLCICQPGYSGSTCHQDLDECQMAQQGPSPCEHGGSCINTPGSFNCLCLPGYTGSRCEADHNECLSQPCHPGSTCLDLLATFHCLCPPGLEGRLCEVEVNECTSNPCLNQAACHDLLNGFQCLCLPGFTGARCEKDMDECSSTPCANGGRCRDQPGAFYCECLPGFEGPHCEKEVDECLSDPCPVGASCLDLPGAFFCLCRPGFTGQLCEVPLCTPNMCQPGQQCQGQEHRAPCLCPDGSPGCVPAEDNCPCHHGHCQRSLCVCDEGWTGPECETELGGCISTPCAHGGTCHPQPSGYNCTCPAGYMGLTCSEEVTACHSGPCLNGGSCSIRPEGYSCTCLPSHTGRHCQTAVDHCVSASCLNGGTCVNKPGTFFCLCATGFQGLHCEEKTNPSCADSPCRNKATCQDTPRGARCLCSPGYTGSSCQTLIDLCARKPCPHTARCLQSGPSFQCLCLQGWTGALCDFPLSCQMAAMSQGIEISGLCQNGGLCIDTGSSYFCRCPPGFQGKLCQDNMNPCEPNPCHHGSTCVPQPSGYVCQCAPGYEGQNCSKVLEACQSQPCHNHGTCTSRPGGFHCACPPGFVGLRCEGDVDECLDRPCHPSGTAACHSLANAFYCQCLPGHTGQRCEVEMDLCQSQPCSNGGSCEITTGPPPGFTCHCPKGFEGPTCSHKALSCGIHHCHNGGLCLPSPKPGSPPLCACLSGFGGPDCLTPPAPPGCGPPSPCLHNGTCTETPGLGNPGFQCTCPPDSPGPRCQRPGASGCEGRGGDGTCDAGCSGPGGDWDGGDCSLGVPDPWKGCPPHSQCWLLFRDGRCHPQCDSEECLFDGYDCEIPLTCIPAYDQYCRDHFHNGHCEKGCNNAECGWDGGDCRPEGEDSEGRPSLALLVVLRPPALDQQLLALARVLSLTLRVGLWVRKDSEGRNMVFPYPGTRAKEELSGARDSSSWERQAPPTQPLGKETESLGAGFVVVMGVDLSRCGPEHPASRCPWDSGLLLRFLAAMAAVGALEPLLPGPLLAAHPQAGTRPPANQLPWPILCSPVVGVLLLALGALLVLQLIRRRRREHGALWLPPGFIRRPQAQQAPHRRRPPLGEDNIGLKALKPEAEVDEDGVAMCSGPEEGEAEETASASRCQLWPLNSSCGELPQAAMLTPPQECESEVLDVDTCGPDGVTPLMSAVFCGGVQSTTGASPQRLGLGNLEPWEPLLDRGACPQAHTVGTGETPLHLAARFSRPTAARRLLEAGANPNQPDRAGRTPLHTAVAADAREVCQLLLASRQTSVDARTEDGTTPLMLAARLAVEDLVEELIAARADVGARDKRGKTALHWAAAVNNARAARSLLQAGADKDAQDSREQTPLFLAAREGAVEVAQLLLELGAARGLRDQAGLAPGDVARQRSHWDLLTLLEGAGPTTQEARAHARTTPGGGAAARCRTLSAGARPRGGGACLQARTWSVDLGARGGKVYARCRSRSGSCGGPTTRGRRFSAGSRGRRGARASQDDWPRDWVALEACGSACSAPIPPPSLTPSPERGSPQVAWGLPVHQEIPLNSVVRNLN

SEQ ID NO: 13 - protein, 2318 aa, Mus musculus
MGLGARGRRRRRRLMALPPPPPPMRALPLLLLLAGLGAAAPPCLDGSPCANGGRCTHQQPSLEAACLCLPGWVGERCQLEDPCHSGPCAGRGVCQSSVVAGTARFSCRCLRGFQGPDCSQPDPCVSRPCVHGAPCSVGPDGRFACACPPGYQGQSCQSDIDECRSGTTCRHGGTCLNTPGSFRCQCPLGYTGLLCENPVVPCAPSPCRNGGTCRQSSDVTYDCACLPGFEGQNCEVNVDDCPGHRCLNGGTCVDGVNTYNCQCPPEWTGQFCTEDVDECQLQPNACHNGGTCFNLLGGHSCVCVNGWTGESCSQNIDDCATAVCFHGATCHDRVASFYCACPMGKTGLLCHLDDACVSNPCHEDAICDTNPVSGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSMCQLDVDECASTPCRNGAKCVDQPDGYECRCAEGFEGTLCERNVDDCSPDPCHHGRCVDGIASFSCACAPGYTGIRCESQVDECRSQPCRYGGKCLDLVDKYLCRCPPGTTGVNCEVNIDDCASNPCTFGVCRDGINRYDCVCQPGFTGPLCNVEINECASSPCGEGGSCVDGENGFHCLCPPGSLPPLCLPANHPCAHKPCSHGVCHDAPGGFRCVCEPGWSGPRCSQSLAPDACESQPCQAGGTCTSDGIGFRCTCAPGFQGHQCEVLSPCTPSLCEHGGHCESDPDRLTVCSCPPGWQGPRCQQDVDECAGASPCGPHGTCTNLPGNFRCICHRGYTGPFCDQDIDDCDPNPCLHGGSCQDGVGSFSCSCLDGFAGPRCARDVDECLSSPCGPGTCTDHVASFTCACPPGYGGFHCEIDLPDCSPSSCFNGGTCVDGVSSFSCLCRPGYTGTHCQYEADPCFSRPCLHGGICNPTHPGFECTCREGFTGSQCQNPVDWCSQAPCQNGGRCVQTGAYCICPPGWSGRLCDIQSLPCTEAAAQMGVRLEQLCQEGGKCIDKGRSHYCVCPEGRTGSHCEHEVDPCTAQPCQHGGTCRGYMGGYVCECPAGYAGDSCEDNIDECASQPCQNGGSCIDLVARYLCSCPPGTLGVLCEINEDDCDLGPSLDSGVQCLHNGTCVDLVGGFRCNCPPGYTGLHCEADINECRPGACHAAHTRDCLQDPGGHFRCVCHPGFTGPRCQIALSPCESQPCQHGGQCRHSLGRGGGLTFTCHCVPPFWGLRCERVARSCRELQCPVGIPCQQTARGPRCACPPGLSGPSCRVSRASPSGATNASCASAPCLHGGSCLPVQSVPFFRCVCAPGWGGPRCETPSAAPEVPEEPRCPRAACQAKRGDQNCDRECNTPGCGWDGGDCSLNVDDPWRQCEALQCWRLFNNSRCDPACSSPACLYDNFDCYSGGRDRTCNPVYEKYCADHFADGRCDQGCNTEECGWDGLDCASEVPALLARGVLVLTVLLPPEELLRSSADFLQRLSAILRTSLRFRLDARGQAMVFPYHRPSPGSESRVRRELGPEVIGSVVMLEIDNRLCLQSAENDHCFPDAQSAADYLGALSAVERLDFPYPLRDVRGEPLEAPEQSVPLLPLLVAGAVFLLIIFILGVMVARRKREHSTLWFPEGFALHKDIAAGHKGRREPVGQDALGMKNMAKGESLMGEVVTDLNDSECPEAKRLKVEEPGMGAEEPEDCRQWTQHHLVAADIRVAPATALTPPQGDADADGVDVNVRGPDGFTPLMLASFCGGALEPMPAEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHLANREITDHLDRLPRDVAQERLHQDIVRLLDQPSGPRSPSGPHGLGPLLCPPGAFLPGLKAVQSGTKKSRRPPGKTGLGPQGTRGRGKKLTLACPGPLADSSVTLSPVDSLDSPRPFSGPPASPGGFPLEGPYATTATAVSLAQLGASRAGPLGRQPPGGCVLSFGLLNPVAVPLDWARLPPPAPPGPSFLLPLAPGPQLLNPGAPVSPQERPPPYLAAPGHGEEYPAAGTRSSPTKARFLRVPSEHPYLTPSPESPEHWASPSPPSLSDWSDSTPSPATATNATASGALPAQPHPISVPSLPQSQTQLGPQPEVTPKRQVMA

SEQ ID NO: 14 - protein, 2471 aa, Rattus norvegicus
MPALRPAALRALLWLWLCGAGPAHALQCRGGQEPCVNEGTCVTYHNGTGYCRCPEGFLGEYCQHRDPCEKNRCQNGGTCVTQAMLGKATCRCAPGFTGEDCQYSTSHPCFVSRPCQNGGTCHMLSWDTYECTCQVGFTGKQCQWTDVCLSHPCENGSTCSSVANQFSCRCPAGITGQKCDADINECDIPGRCQHGGTCLNLPGSYRCQCPQRFTGQHCDSPYVPCAPSPCVNGGTCRQTGDFTSECHCLPGFEGSNCERNIDDCPNHKCQNGGVCVDGVNTYNCRCPPQWTGQFCTEDVDECLLQPNACQNGGTCTNRNGGYGCVCVNGWSGDDCSENIDDCAFASCTPGSTCIDRVASFSCLCPEGKAGLLCHLDDACISNPCHKGALCDTNPLNGQYICTCPQAYKGADCTEDVDECAMANSNPCEHAGKCVNTDGAFHCECLKGYAGPRCEMDINECHSDPCQNDATCLDKIGGFTCLCMPGFKGVHCELEVNECQSNPCVNNGQCVDKVNRFQCLCPPGFTGPVCQIDIDDCSSTPCLNGAKCIDHPNGYECQCATGFTGTLCDENIDNCDPDPCHHGQCQDGIDSYTCICNPGYMGAICSDQIDECYSSPCLNDGRCIDLVNGYQCNCQPGTSGLNCEINFDDCASNPCLHGACVDGINRYSCVCSPGFTGQRCNIDIDECASNPCRKDATCINDVNGFRCMCPEGPHHPSCYSQVNECLSSPCIHGNCTGGLSGYKCLCDAGWVGINCEVDKNECLSNPCQNGGTCNNLVNGYRCTCKKGFKGYNCQVNIDECASNPCLNQGTCLDDVSGYTCHCMLPYTGKNCQTVLAPCSPNPCENAAVCKEAPNFESFTCLCAPGWQGQRCTVDVDECVSKPCMNNGICHNTQGSYMCECPPGFSGMDCEEDINDCLANPCQNGGSCVDKVNTFSCLCLPGFVGDKCQTDMNECLSEPCKNGGTCSDYVNSYTCTCPAGFHGVHCENNIDECTESSCFNGGTCVDGINSFSCLCPVGFTGPFCLHDINECSSNPCLNSGTCVDGLGTYRCTCPLGYTGKNCQTLVNLCSPSPCKNKGTCAQEKARPRCLCPPGWDGAYCDVLNVSCKAAALQKGVPVEHLCQHSGICINAGNTHHCQCPLGYTGSYCEEQLDECASNPCQHGATCSDFIGGYRCECVPGYQGVNCEYEVDECQNQPCQNGGTCIDLVNHFKCSCPPGTRGLLCEENIDDCAGAPHCLNGGQCVDRIGGYSCRCLPGFAGERCEGDINECLSNPCSSEGSLDCIQLKNNYQCVCRSAFTGRHCETFLDVCPQKPCLNGGTCAVASNVPDGFICRCPPGFSGARCQSSCGQVKCRRGEQCVHTASGPHCFCPNHKDCESGCASNPCQHGGTCYPQRQPPYYSCRCSPPFWGSHCESYTAPTSTPPATCLSQYCADKARDGICDEACNSHACQWDGGDCSLTMEDPWANCTSSLRCWEYINNQCDELCNTAECLFDNFECQRNSKTCKYDKYCADHFKDNHCDKGCNNEECGWDGLDCAADQPENLAEGILVIVVLLPPEQLLQDSRSFLRALGTLLHTNLRIKQDSQGALMVYPYYGEKSAAMKKQKVARRSLPDEQEQEIIGSKVFLEIDNRQCVQDSDQCFKNTDAAAALLASHAIQGTLSYPLVSVVSESEDPRNTPLLYLLAVAVVIILFLILLGVIMAKRKRKHGFLWLPEGFTLRRDSSNHKRREPVGQDAVGLKNLSVQVSEANLIGSTTSEHWGDDEGPQPKKAKAEDDEALLSEDDPVDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVLCGPNRSFLSLKHTPMGKKARRPNTKSTMPTSLPNLAKEAKDVKGSRRKKCLNEKVQLSESSVTLSPVDSLESPHTYVSDATSSPMITSPGILQASPTPLLAAAPAAPVHAQHALSFSNLHEMQPLRPGASTVLPSVSQLLSHHHIVPPGSGSAGSLGRLHSVPVPSDWMNRVEMSETQYSEMFGMVLAPAEGTHPGMAAPQSRAPEGKPIPTQREPLPPIVTFQLIPKGSLAQAAGAPQTQSGCPPAVAGPLPSMYQIPEMARLPSVAFPPTMMPQQEGQVAQTIVPTYHPFPASVGKYPTPPSQHSYASSNAAERTPNHGGHLQGEHPYLTPSPESPDQWSSSSPHSASDWSDVTTSPTPGGGGGGQRGPGTHMSEPPHSNMQVYA

SEQ ID NO: 15 - protein, 2319 aa, Rattus norvegicus
MGPGARGRRRRRRLMALPPPPPPMRALPLLLLLLAGLGAAAPPCLDGSPCANGGRCTHQQPSREAACLCLPGWVGERCQLEDPCHSGPCAGRGVCQSSVVAGVARFSCRCLRGFRGPDCSLPDPCFSSPCAHGAPCSVGSDGRYACACPPGYQGRNCRSDIDECRAGASCRHGGTCINTPGSFHCLCPLGYTGLLCENPIVPCAPSPCRNGGTCRQSSDVTYDCACLPGFEGQNCEVNVDDCPGHRCLNGGTCVDGVNTYNCQCPPEWTGQFCTEDVDECQLQPNACHNGGTCFNLLGGHSCVCVNGWTGESCSQNIDDCATAVCFHGATCHDRVASFYCACPMGKTGLLCHLDDACVSNPCHEDAICDTNPVSGRAICTCPPGFTGGACDQDVDECSIGANPCEHLGRCVNTQGSFLCQCGRGYTGPRCETDVNECLSGPCRNQATCLDRIGQFTCICMAGFTGTFCEVDIDECQSSPCVNGGVCKDRVNGFSCTCPSGFSGSTCQLDVDECASTPCRNGAKCVDQPDGYECRCAEGFEGTLCERNVDDCSPDPCHHGRCVDGIASFSCACAPGYTGIRCESQVDECRSQPCRYGGKCLDLVDKYLCRCPPGTTGVNCEVNIDDCASNPCTFGVCRDGINRYDCVCQPGFTGPLCNVEINECASSPCGEGGSCVDGENGFHCLCPPGSLPPLCLPANHPCAHKPCSHGVCHDAPGGFQCVCDPGWSGPRCSQSLAPDACESQPCQAGGTCTSDGIGFHCTCAPGFQGHQCEVLSPCTPSLCEHGGHCESDPDQLTVCSCPPGWQGPRCQQDVDECAGASPCGPHGTCTNLPGSFRCICHGGYTGPFCDQDIDDCDPNPCLNGGSCQDGVGSFSCSCLSGFAGPRCARDVDECLSSPCGPGTCTDHVASFTCTCPPGYGGFHCETDLLDCSPSSCFNGGTCVDGVNSFSCLCRPGYTGTHCQYKVDPCFSRPCLHGGICNPTHSGFECTCREGFTGNQCQNPVDWCSQAPCQNGGRCVQTGAYCICPPEWSGPLCDIPSLPCTEAAAHMGVRLEQLCQAGGQCIDKDHSHYCVCPEGRMGSHCEQEVDPCTAQPCQHGGTCRGYMGGYVCECPTGYSGDSCEDDVDECASQPCQNGGSCIDLVAHYLCSCPPGTLGVLCEINEDDCGPGPSLDSGLRCLHNGTCVDLVGGFRCNCPPGYTGLHCEADINECRPGTCHAAHTRDCLQDPGGHFRCICLPGFTGPRCQTALFPCESQPCQHGGQCRPSLGRGGGLTFTCHCVQPFWGLRCERVARSCRELQCPVGIPCQQTARGPRCACPPGLSGPSCRVSRASPSGATNTSCAATPCLHGGSCLPVQSVPFFRCVCAPGWGGPRCETPSAAPEVPEEPRCPRAACQAKRGDQNCDRECNSPGCGWDGGDCSLNVDDPWRQCEALQCWRLFNNSRCDPACSSPACLYDNFDCYSGGRDRTCNPVYKKYCADHFADGRCDQGCNTEECGWDGLDCASEVPALLARGVLVLTVLLPPEELLRSSADFLQRLSAILRTSLRFRLDARGQAMVFPYHRPSPGSESRVRRELGPEVIGSVVMLEIDNRLCLKSAENDHCFPDAQSAADYLGALSAVERLDFPYPLRDVRGEPLEPPEQSVPLLPLLVAGAVFLLVIFVLGVMVARRKREHSTLWFPEGFALHKDIAAGHKGRREPVGQDALGMKNMTKGESLMGEVATDWNDSECPEAKRLKVEEPGMGAEEPVDCRQWTQHHLVAADIRVAPAMALTPPQGDADADGMDVNVRGPDGFTPLMLASFCGGALEPMPAEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPSGPHGLGPLLCPPGAFLPGLKAVQSGTKKSRRPPGKTGLGPQGTRGRGKKLTLACPGPLADSSVTLSPVDSLDSPRPFGGPPASPGGFPLEGPYATTATTVSLAQLGASRAGPLGRQPPGGCVLSLGLLNPVAVPLDWARLPPPAPPGPSFLLPLAPGSQLLNPATPVSPHERPPPYLAAPGHGEEYPAAGTHSSPTKARFLRVPSEHPYLTPSPESPEHWASPSPPSLSDWSDSTPSPATATSATAAGALPAQPHPISVPSLPQSQTQLGPQPEVTPKRQVMA

SEQ ID NO: 16 - protein, 333 aa, synthetic construct
  REGION 1..333: note = synthetic amino acid sequence
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKL

SEQ ID NO: 17 - protein, 370 aa, synthetic construct
  REGION 1..370: note = synthetic amino acid sequence
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRRQLCIQKL

SEQ ID NO: 18 - protein, 9 aa, synthetic construct
  REGION 1..9: note = synthetic amino acid sequence
RMFPNAPYL

SEQ ID NO: 19 - protein, 10 aa, synthetic construct
  REGION 1..10: note = synthetic amino acid sequence
KLVVVGAGGV

SEQ ID NO: 20 - protein, 10 aa, synthetic construct
  REGION 1..10: note = synthetic amino acid sequence
KLVVVGAVGV

SEQ ID NO: 21 - protein, 10 aa, synthetic construct
  REGION 1..10: note = synthetic amino acid sequence
KLVVVGACGV

SEQ ID NO: 22 - protein, 10 aa, synthetic construct
  REGION 1..10: note = synthetic amino acid sequence
KLVVVGADGV

SEQ ID NO: 23 - protein, 9 aa, synthetic construct
  REGION 1..9: note = synthetic amino acid sequence
VVGAVGVGK

SEQ ID NO: 24 - protein, 9 aa, synthetic construct
  REGION 1..9: note = synthetic amino acid sequence
VVGACGVGK

SEQ ID NO: 25 - protein, 9 aa, synthetic construct
  REGION 1..9: note = synthetic amino acid sequence
VVGAGGVGK

SEQ ID NO: 26 - protein, 9 aa, synthetic construct
  REGION 1..9: note = synthetic amino acid sequence
KITDFGLAK

SEQ ID NO: 27 - protein, 9 aa, synthetic construct
  REGION 1..9: note = synthetic amino acid sequence
KITDFGRAK

SEQ ID NO: 28 - protein, 9 aa, synthetic construct
  REGION 1..9: note = synthetic amino acid sequence
VLQELNVTV

SEQ ID NO: 29 - protein, 9 aa, synthetic construct
  REGION 1..9: note = synthetic amino acid sequence
EADPTGHSY

SEQ ID NO: 30 - protein, 9 aa, synthetic construct
  REGION 1..9: note = synthetic amino acid sequence
FLWGPRALV

SEQ ID NO: 31 - protein, 9 aa, synthetic construct
  REGION 1..9: note = synthetic amino acid sequence
LLGRNSFEV

SEQ ID NO: 32 - protein, 10 aa, synthetic construct
  REGION 1..10: note = synthetic amino acid sequence
STTPPPGTRV

SEQ ID NO: 33 - protein, 9 aa, synthetic construct
  REGION 1..9: note = synthetic amino acid sequence
RMPEAAPPV

SEQ ID NO: 34 - protein, 11 aa, synthetic construct
  REGION 1..11: note = synthetic amino acid sequence
GLAPPQHLIRV

SEQ ID NO: 35 - protein, 10 aa, synthetic construct
  REGION 1..10: note = synthetic amino acid sequence
ELAGIGILTV

SEQ ID NO: 36 - protein, 10 aa, synthetic construct
  REGION 1..10: note = synthetic amino acid sequence
EAAGIGILTV

SEQ ID NO: 37 - protein, 9 aa, synthetic construct
  REGION 1..9: note = synthetic amino acid sequence
IMDQVPFSV

SEQ ID NO: 38 - protein, 9 aa, synthetic construct
  REGION 1..9: note = synthetic amino acid sequence
KTWGQYWQV

SEQ ID NO: 39 - protein, 9 aa, synthetic construct
  REGION 1..9: note = synthetic amino acid sequence
YLEPGPVTV

SEQ ID NO: 40 - protein, 9 aa, synthetic construct
  REGION 1..9: note = synthetic amino acid sequence
YLEPGPVTA

SEQ ID NO: 41 - protein, 9 aa, synthetic construct
  REGION 1..9: note = synthetic amino acid sequence
ITDQVPFSV

SEQ ID NO: 42 - protein, 9 aa, synthetic construct
  REGION 1..9: note = synthetic amino acid sequence
NLVPMVATV

SEQ ID NO: 43 - protein, 9 aa, synthetic construct
  REGION 1..9: note = synthetic amino acid sequence
AIIRILQQL

SEQ ID NO: 44 - protein, 9 aa, synthetic construct
  REGION 1..9: note = synthetic amino acid sequence
VLHDDLLEA

SEQ ID NO: 45 - protein, 9 aa, synthetic construct
  REGION 1..9: note = synthetic amino acid sequence
VLRDDLLEA

SEQ ID NO: 46 - protein, 9 aa, synthetic construct
  REGION 1..9: note = synthetic amino acid sequence
SLLMWITQV

SEQ ID NO: 47 - protein, 10 aa, synthetic construct
  REGION 1..10: note = synthetic amino acid sequence
LLDFVRFMGV

SEQ ID NO: 48 - protein, 9 aa, synthetic construct
  REGION 1..9: note = synthetic amino acid sequence
FMNKFIYEI

SEQ ID NO: 49 - protein, 9 aa, synthetic construct
  REGION 1..9: note = synthetic amino acid sequence
KIFGSLAFL

SEQ ID NO: 50 - protein, 9 aa, synthetic construct
  REGION 1..9: note = synthetic amino acid sequence
GVLPALPQV

SEQ ID NO: 51 - protein, 9 aa, synthetic construct
  REGION 1..9: note = synthetic amino acid sequence
TMTRVLQGV

SEQ ID NO: 52 - protein, 9 aa, synthetic construct
  REGION 1..9: note = synthetic amino acid sequence
FLLTRILTI

SEQ ID NO: 53 - protein, 9 aa, synthetic construct
  REGION 1..9: note = synthetic amino acid sequence
ILAKFLHWL

SEQ ID NO: 54 - protein, 9 aa, synthetic construct
  REGION 1..9: note = synthetic amino acid sequence
RLVDDFLLV

SEQ ID NO: 55 - protein, 9 aa, synthetic construct
  REGION 1..9: note = synthetic amino acid sequence
LLLTVLTVV

SEQ ID NO: 56 - protein, 9 aa, synthetic construct
  REGION 1..9: note = synthetic amino acid sequence
FLRNFSLML

SEQ ID NO: 57 - protein, 9 aa, synthetic construct
  REGION 1..9: note = synthetic amino acid sequence
YMDGTMSQV

SEQ ID NO: 58 - protein, 9 aa, synthetic construct
  REGION 1..9: note = synthetic amino acid sequence
YLLPAIVHI

SEQ ID NO: 59 - protein, 9 aa, synthetic construct
  REGION 1..9: note = synthetic amino acid sequence
FLSELTQQL

SEQ ID NO: 60 - protein, 10 aa, synthetic construct
  REGION 1..10: note = synthetic amino acid sequence
ALYVDSLFFL

SEQ ID NO: 61 - protein, 5 aa, synthetic construct
  REGION 1..5: note = synthetic amino acid sequence
SYAMS

SEQ ID NO: 62 - protein, 17 aa, synthetic construct
  REGION 1..17: note = synthetic amino acid sequence
QIDPWGQETLYADSVKG

SEQ ID NO: 63 - protein, 7 aa, synthetic construct
  REGION 1..7: note = synthetic amino acid sequence
LTGRFDY

SEQ ID NO: 64 - protein, 11 aa, synthetic construct
  REGION 1..11: note = synthetic amino acid sequence
RASQSISSYLN

SEQ ID NO: 65 - protein, 7 aa, synthetic construct
  REGION 1..7: note = synthetic amino acid sequence
SASQLQS

SEQ ID NO: 66 - protein, 9 aa, synthetic construct
  REGION 1..9: note = synthetic amino acid sequence
QQGPGTPNT

SEQ ID NO: 67 - protein, 241 aa, synthetic construct
  REGION 1..241: note = synthetic amino acid sequence
EVQLLESGGGLVQPGGSLRLSCAASGFTFSSYAMSWVRQAPGKGLEWVSQIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASQLQSGVPSRFSGSGSGTDFTLTISSLQPEDFATYYCQQGPGTPNTFGQGTKVEIKRA

SEQ ID NO: 68 - protein, 241 aa, synthetic construct
  REGION 1..241: note = synthetic amino acid sequence
DIQMTQSPSSLSASVGDRVTITCRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGGGASEVQLVESGGGLVQPGGSLRLSCAASGFNINGSYIHWVRQAPGKGLEWVAYIDPETGYSRYADSVKGRFTISADTSKNTAYLQMNSLRAEDTAVYYCSRDSASDAMDVWGQGTLVTVSS

SEQ ID NO: 69 - protein, 241 aa, synthetic construct
  REGION 1..241: note = synthetic amino acid sequence
DIQMTQSPSSLSASVGDRVTIACRASQDVNTAVAWYQQKPGKAPKLLIYSASFLYSGVPSRFSGSRSGTDFTLTISSLQPEDFATYYCQQYYYYPPTFGQGTKVEIKRTGGGSGGGGSGGGASEVQLVESGGGLVQPGGSLRLSCAASGFHINGSYIHWVRQAPGKGLKWVAYIDPETGYSRYADSVKGRFAISADMSKNTAYLQMNSLRAEDTAVYYCSRDSASDAMDVWGQGTLVTVSS

SEQ ID NO: 70 - protein, 243 aa, synthetic construct
  REGION 1..243: note = synthetic amino acid sequence
DIQMTQSPSSLSASVGDRVTITCRASQDVNTAVAWYQQKPGKAPKLLIYSASFLYSGVPSRFSGSRSGTDFTLTISSLQPEDFATYYCQQYYSYPPTFGQGTKVEIKRTGGGSGGGGSGGGASEVQLVESGGGLVQPGGSLRLSCAASGFNITSSYIHWVRQAPGKGLEWVAYISPEDGYARHADSVKGRFTISADTSKNTAYLQMNSLRAEDTAVYYCSRDDTYYYSAMDVWGQGTLVTVSS

SEQ ID NO: 71 - protein, 9 aa, synthetic construct
  REGION 1..9: note = synthetic amino acid sequence
HLSPIDCEV

SEQ ID NO: 72 - protein, 9 aa, synthetic construct
  REGION 1..9: note = synthetic amino acid sequence
LTSMWSPAV

SEQ ID NO: 73 - protein, 9 aa, synthetic construct
  REGION 1..9: note = synthetic amino acid sequence
HLDHPHPAV

SEQ ID NO: 74 - protein, 9 aa, synthetic construct
  REGION 1..9: note = synthetic amino acid sequence
SLSVMSSNV

SEQ ID NO: 75 - protein, 9 aa, synthetic construct
  REGION 1..9: note = synthetic amino acid sequence
MMAWSDNKI

SEQ ID NO: 76 - protein, 9 aa, synthetic construct
  REGION 1..9: note = synthetic amino acid sequence
TQIGIEWNL

SEQ ID NO: 77 - protein, 9 aa, synthetic construct
  REGION 1..9: note = synthetic amino acid sequence
CLFEMQDPA

SEQ ID NO: 78 - protein, 9 aa, synthetic construct
  REGION 1..9: note = synthetic amino acid sequence
YLSHPSCNV

SEQ ID NO: 79 - protein, 9 aa, synthetic construct
  REGION 1..9: note = synthetic amino acid sequence
GIEWNLSPV

SEQ ID NO: 80 - protein, 9 aa, synthetic construct
  REGION 1..9: note = synthetic amino acid sequence
GLGCSPASI

SEQ ID NO: 81 - protein, 9 aa, synthetic construct
  REGION 1..9: note = synthetic amino acid sequence
RQAFEFPQI

SEQ ID NO: 82 - protein, 9 aa, synthetic construct
  REGION 1..9: note = synthetic amino acid sequence
GMWTDTFEF

SEQ ID NO: 83 - protein, 9 aa, synthetic construct
  REGION 1..9: note = synthetic amino acid sequence
YLNWQDTAV

SEQ ID NO: 84 - protein, 9 aa, synthetic construct
  REGION 1..9: note = synthetic amino acid sequence
VLWGPITQI

SEQ ID NO: 85 - protein, 9 aa, synthetic construct
  REGION 1..9: note = synthetic amino acid sequence
TLDHTGVVV

SEQ ID NO: 86 - protein, 9 aa, synthetic construct
  REGION 1..9: note = synthetic amino acid sequence
TMGVWLHIA

SEQ ID NO: 87 - protein, 9 aa, synthetic construct
  REGION 1..9: note = synthetic amino acid sequence
KVWVQGHYL

SEQ ID NO: 88 - protein, 9 aa, synthetic construct
  REGION 1..9: note = synthetic amino acid sequence
KINCNNFRL

SEQ ID NO: 89 - protein, 56 aa, synthetic construct
  REGION 1..56: note = synthetic amino acid sequence
IPYKIEAVKSEPVEPPLPSQLHLMYVAAAAFVLLFFVGCGVLLSRKRRRQLCIQKL

SEQ ID NO: 90 - protein, 120 aa, synthetic construct
  REGION 1..120: note = synthetic amino acid sequence
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDC

SEQ ID NO: 91 - protein, 152 aa, synthetic construct
  REGION 1..152: note = synthetic amino acid sequence
AAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLP

SEQ ID NO: 92 - protein, 23 aa, synthetic construct
  REGION 1..23: note = synthetic amino acid sequence
HLMYVAAAAFVLLFFVGCGVLLS

SEQ ID NO: 93 - protein, 306 aa, synthetic construct
  REGION 1..306: note = synthetic amino acid sequence
PPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 94 - protein, 358 aa, synthetic construct
  REGION 1..358: note = synthetic amino acid sequence
PCVGSNPCYNQGTCEPTSENPFYRCLCPAKFNGLLCHILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLS

SEQ ID NO: 95 - protein, 37 aa, synthetic construct
  REGION 1..37: note = synthetic amino acid sequence
PCVGSNPCYNQGTCEPTSENPFYRCLCPAKFNGLLCH

SEQ ID NO: 96 - protein, 37 aa, synthetic construct
  REGION 1..37: note = synthetic amino acid sequence
DINECVLSPCRHGASCQNTHGGYRCHCQAGYSGRNCE

SEQ ID NO: 97 - protein, 37 aa, synthetic construct
  REGION 1..37: note = synthetic amino acid sequence
DIDDCRPNPCHNGGSCTDGINTAFCDCLPGFRGTFCE

SEQ ID NO: 98 - protein, 37 aa, synthetic construct
  REGION 1..37: note = synthetic amino acid sequence
DINECASDPCRNGANCTDCVDSYTCTCPAGFSGIHCE

SEQ ID NO: 99 - protein, 32 aa, synthetic construct
  REGION 1..32: note = synthetic amino acid sequence
TESSCFNGGTCVDGINSFTCLCPPGFTGSYCQ

SEQ ID NO: 100 - protein, 37 aa, synthetic construct
  REGION 1..37: note = synthetic amino acid sequence
DVNECDSQPCLHGGTCQDGCGSYRCTCPQGYTGPNCQ

SEQ ID NO: 101 - protein, 27 aa, synthetic construct
  REGION 1..27: note = synthetic amino acid sequence
DSSPCKNGGKCWQTHTQYRCECPSGWT

SEQ ID NO: 102 - protein, 36 aa, synthetic construct
  REGION 1..36: note = synthetic amino acid sequence
LVDECSPSPCQNGATCTDYLGGYSCKCVAGYHGVNC

SEQ ID NO: 103 - protein, 36 aa, synthetic construct
  REGION 1..36: note = synthetic amino acid sequence
IDECLSHPCQNGGTCLDLPNTYKCSCPRGTQGVHCE

SEQ ID NO: 104 - protein, 28 aa, synthetic construct
  REGION 1..28: note = synthetic amino acid sequence
CFNNGTCVDQVGGYSCTCPPGFVGERCE

SEQ ID NO: 105 - protein, 39 aa, synthetic construct
  REGION 1..39: note = synthetic amino acid sequence
DVNECLSNPCDARGTQNCVQRVNDFHCECRAGHTGRRCE

SEQ ID NO: 106 - protein, 10 aa, synthetic construct
  REGION 1..10: note = synthetic amino acid sequence
GRRRRELDPM

SEQ ID NO: 107 - protein, 9 aa, synthetic construct
  REGION 1..9: note = synthetic amino acid sequence
RQRRELDPM

SEQ ID NO: 108 - protein, 8 aa, synthetic construct
  REGION 1..8: note = synthetic amino acid sequence
KIEAVKSE

SEQ ID NO: 109 - protein, 8 aa, synthetic construct
  REGION 1..8: note = synthetic amino acid sequence
KIEAVQSE

SEQ ID NO: 110 - protein, 8 aa, synthetic construct
  REGION 1..8: note = synthetic amino acid sequence
VGCGVLLS

SEQ ID NO: 111 - protein, 7 aa, synthetic construct
  REGION 1..7: note = synthetic amino acid sequence
GCGVLLS

SEQ ID NO: 112 - protein, 248 aa, synthetic construct
  REGION 1..248: note = synthetic amino acid sequence
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGGPADALDDFDLDMLPADALDDFDLDMLPADALDDFDLDMLPG

SEQ ID NO: 113 - protein, 211 aa, synthetic construct
  REGION 1..211: note = synthetic amino acid sequence
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 114 - protein, 115 aa, synthetic construct
  REGION 1..115: note = synthetic amino acid sequence
PKKKRKVDALDDFDLDMLGSDALDDFDLDMLGSDALDDFDLDMLGSDALDDFDLDMLGSGGSGGSGGSLEIEAAFLERENTALETRVAELRQRVQRLRNRVSQYRTRYGPLGGGK

SEQ ID NO: 115 - protein, 5 aa, synthetic construct
  REGION 1..5: note = synthetic amino acid sequence
GGGGS

SEQ ID NO: 116 - protein, 282 aa, synthetic construct
  REGION 1..282: note = synthetic amino acid sequence
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLLDGTYIPTFGRGTSLIVHPGSADDAKKDAAKKDGKSMSIGLLCCAALSLLWAGPVNAGVTQTPKFQVLKTGQSMTLQCAQDMNHEYMSWYRQDPGMGLRLIHYSVGAGTTDRGEVPNGYNVSRSTIEDFPLRLLSAAPSQTSVYFCASSYVGDTGELFFGEGSRLTVL

SEQ ID NO: 117 - protein, 9 aa, synthetic construct
  REGION 1..9: note = synthetic amino acid sequence
YPYDVPDYA

SEQ ID NO: 118 - protein, 8 aa, synthetic construct
  REGION 1..8: note = synthetic amino acid sequence
DYKDDDDK

SEQ ID NO: 119 - protein, 10 aa, synthetic construct
  REGION 1..10: note = synthetic amino acid sequence
EQKLISEEDL

SEQ ID NO: 120 - protein, 5 aa, synthetic construct
  REGION 1..5: note = synthetic amino acid sequence
HHHHH

SEQ ID NO: 121 - protein, 6 aa, synthetic construct
  REGION 1..6: note = synthetic amino acid sequence
HHHHHH

SEQ ID NO: 122 - protein, 8 aa, synthetic construct
  REGION 1..8: note = synthetic amino acid sequence
WSHPQFEK

SEQ ID NO: 123 - protein, 5 aa, synthetic construct
  REGION 1..5: note = synthetic amino acid sequence
RYIRS

SEQ ID NO: 124 - protein, 4 aa, synthetic construct
  REGION 1..4: note = synthetic amino acid sequence
FHHT

SEQ ID NO: 125 - protein, 17 aa, synthetic construct
  REGION 1..17: note = synthetic amino acid sequence
WEAAAREACCRECCARA

SEQ ID NO: 126 - protein, 7 aa, synthetic construct
  REGION 1..7: note = synthetic amino acid sequence
PKKKRKV

SEQ ID NO: 127 - protein, 16 aa, synthetic construct
  REGION 1..16: note = synthetic amino acid sequence
KRPAATKKAGQAKKKK

SEQ ID NO: 128 - protein, 8 aa, synthetic construct
  REGION 1..8: note = synthetic amino acid sequence
MVPKKKRK

SEQ ID NO: 129 - protein, 17 aa, synthetic construct
  REGION 1..17: note = synthetic amino acid sequence
MAPKKKRKVGIHGVPAA

SEQ ID NO: 130 - skipped

SEQ ID NO: 131 - protein, 142 aa, synthetic construct
  REGION 1..142: note = synthetic amino acid sequence
PNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 132 - protein, 138 aa, synthetic construct
  REGION 1..138: note = synthetic amino acid sequence
PYIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSS

SEQ ID NO: 133 - protein, 177 aa, synthetic construct
  REGION 1..177: note = synthetic amino acid sequence
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 134 - protein, 173 aa, synthetic construct
  REGION 1..173: note = synthetic amino acid sequence
EDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNS

(19) United States
(12) Patent Application Publication
(10) Pub. No.: US 2025/0304698 A1
(43) Pub. Date: Oct. 2, 2025

(54) PROTEOLYTICALLY CLEAVABLE CHIMERIC POLYPEPTIDES AND METHODS OF USE THEREOF

(71) Applicant: The Regents of the University of California, Oakland, CA (US)

(72) Inventors: Wendell A. Lim, San Francisco, CA (US); Kole T. Roybal, San Francisco, CA (US); Jasper Z. Williams, San Francisco, CA (US)

(21) Appl. No.: 19/234,169

(22) Filed: Jun. 10, 2025

Related U.S. Application Data
(62) Division of application No. 17/841,595, filed on Jun. 15, 2022, now Pat. No. 12,358,988.
Application No. 17/841,595 is a division of application No. 16/325,657, filed on Feb. 14, 2019, now Pat. No. 11,401,332.
(60) Provisional application No. 62/378,614, filed on Aug. 23, 2016.

(51) Int. Cl. C07K 16/28; A61K 39/00; A61K 39/395; A61K 40/11; A61K 40/31; A61K 40/32; A61K 40/42; A61P 35/00; C07K 14/705; C07K 14/725; C07K 14/74; C07K 16/30; C12N 15/63

(52) U.S. Cl. CPC C07K 16/2833; A61K 39/39558; A61K 40/11; A61K 40/31; A61K 40/32; A61K 40/4202; A61K 40/4205; A61K 40/421; A61K 40/4211; A61K 40/4213; A61K 40/4224; A61K 40/4243; A61K 40/4269; A61K 40/4272; A61P 35/00; C07K 14/705; C07K 14/7051; C07K 14/70539; C07K 16/2803; C07K 16/2809; C07K 16/30; C12N 15/63; A61K 2039/505; A61K 2239/28; C07K 2317/14; C07K 2317/31; C07K 2317/32; C07K 2317/622; C07K 2319/00; C07K 2319/50; C07K 2319/60; C07K 2319/715; C07K 2319/80

(57) ABSTRACT

The instant disclosure provides chimeric polypeptides which modulate various cellular processes following a cleavage event induced upon binding of a specific binding member of the polypeptide with its binding partner. Methods of using chimeric polypeptides to modulate cellular functions, including e.g., induction of gene expression, are also provided. Nucleic acids encoding the subject chimeric polypeptides and associated expression cassettes and vectors as well as cells that contain such nucleic acids and/or expression cassettes and vectors are provided. Also provided, are methods of treating a subject using the described components and methods as well as kits for practicing the subject methods.

### CROSS-REFERENCE

This application is a divisional of U.S. application Ser. No. 17/841,595, filed on Jun. 15, 2022, which is a divisional of U.S. application Ser. No. 16/325,657, filed on Feb. 14, 2019, now issued as U.S. Pat. No. 11,401,332, which is a § 371 national phase of International Application No. PCT/US2017/048040, filed on Aug. 22, 2017, which claims the benefit of U.S. Provisional Patent Application No. 62/378,614, filed Aug. 23, 2016, which applications are incorporated herein by reference in their entireties.

### STATEMENT REGARDING FEDERALLY SPONSORED RESEARCH

This invention was made with government support under grant no. R01 CA196277, awarded by the National Institutes of Health. The government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, “UCSF-544DIV2_SEQLIST.xml” created on Jun. 10, 2025, and having a size of 201,798 bytes. The contents of the Sequence Listing XML are incorporated by reference herein in their entirety.

### INTRODUCTION

Conventionally, control of cellular behaviors and activities has been achieved through the use of inducible expression constructs driving expression of a protein that, when expressed, alters cellular behavior and/or activity. In the research setting, inducible expression systems have greatly advanced our understanding of many areas of the life sciences, including cell biology, molecular biology, genetics, biochemistry and others. Well-studied inducible cell systems (e.g., chemically inducible, optically inducible, etc.) generally affect cell behaviors and activities globally and/or require a user-provided input to restrict a change in activity to particular cells of a population or control the system, e.g., toggling the system “on” or “off”. Cellular engineering has recently provided the ability to attempt to reprogram cells to detect signals in their environments, e.g., as provided by neighboring cells, and autonomously transduce such signaling inputs into desired behavioral or activity outputs.

### SUMMARY

The instant disclosure provides chimeric polypeptides which modulate various cellular processes following a cleavage event induced upon binding of a specific binding member of the polypeptide with its binding partner. Methods of using chimeric polypeptides to modulate cellular functions, including e.g., induction of gene expression, are also provided. Nucleic acids encoding the subject chimeric polypeptides and associated expression cassettes and vectors as well as cells that contain such nucleic acids and/or expression cassettes and vectors are provided. Also provided, are methods of treating a subject using the described components and methods as well as kits for practicing the subject methods.

Aspects of the instant disclosure include a chimeric polypeptide comprising, from N-terminal to C-terminal and in covalent linkage: a) an extracellular domain comprising a specific binding member that specifically binds to a peptide-major histocompatibility complex (peptide-MHC); b) a proteolytically cleavable Notch receptor polypeptide comprising one or more proteolytic cleavage sites; and c) an intracellular domain comprising a transcriptional activator or repressor, wherein binding of the specific binding member to the peptide-MHC induces cleavage of the Notch receptor polypeptide at the one or more proteolytic cleavage sites, thereby releasing the intracellular domain.

In some embodiments, the specific binding member comprises an antibody. In some embodiments, the antibody is a nanobody, a diabody, a triabody, or a minibody, a F(ab′)₂ fragment, a Fab fragment, a single chain variable fragment (scFv) or a single domain antibody (sdAb).

In some embodiments, the specific binding member specifically binds a peptide-MHC comprising an intracellular cancer antigen peptide. In some embodiments, the intracellular cancer antigen peptide is a WT1 peptide or a NY-ESO peptide.

In some embodiments, the Notch receptor polypeptide comprises, at its N-terminus, one or more epidermal growth factor (EGF) repeats. In some embodiments, the Notch receptor polypeptide comprises, at its N-terminus, 2 to 11 EGF repeats. In some embodiments, the Notch receptor polypeptide comprises a synthetic linker. In some embodiments, the Notch receptor polypeptide comprises a synthetic linker between the one or more EGF repeats and the one or more proteolytic cleavage sites. In some embodiments, the Notch receptor polypeptide has a length from 50 amino acids to 1000 amino acids. In some embodiments, the Notch receptor polypeptide has a length from 300 amino acids to 400 amino acids. In some embodiments, the one or more proteolytic cleavage sites comprises an S2 proteolytic cleavage site, an S3 proteolytic cleavage site or a combination thereof. In some embodiments, the one or more proteolytic cleavage sites comprises an S2 proteolytic cleavage site that is an ADAM family type protease cleavage site, such as e.g., an ADAM-17-type protease cleavage site comprising an Ala-Val dipeptide sequence. In some embodiments, the one or more proteolytic cleavage sites comprises an S3 proteolytic cleavage site that is a gamma-secretase (γ-secretase) cleavage site comprising a Gly-Val dipeptide sequence. In some embodiments, the one or more proteolytic cleavage sites further comprises an S1 proteolytic cleavage site. In some embodiments, the S1 proteolytic cleavage site is a furin-like protease cleavage site comprising the amino acid sequence Arg-X-(Arg/Lys)-Arg (SEQ ID NO: 130), where X is any amino acid. In some embodiments, the Notch receptor polypeptide lacks an S1 proteolytic cleavage site. In some embodiments, the Notch receptor polypeptide has at least 85% amino acid sequence identity to a sequence provided in FIG. 31A-32B. In some embodiments, the Notch receptor polypeptide has at least 85% amino acid sequence identity to the sequence provided in FIG. 31A or the sequence provided in FIG. 31B.

Aspects of the instant disclosure include a nucleic acid encoding any of the above described chimeric polypeptides.

In some embodiments, the nucleic acid further comprises a transcriptional control element responsive to the transcriptional activator or repressor operably linked to a nucleic acid sequence encoding a polypeptide of interest (POI). In some embodiments, the POI is a heterologous polypeptide selected from the group consisting of: a reporter protein, a chimeric antigen receptor (CAR), an antibody, a chimeric bispecific binding member, an engineered T cell receptor (TCR) and an innate-immune response inducer.

Aspects of the instant disclosure include a recombinant expression vector comprising any of the above described nucleic acids.

Aspects of the instant disclosure include a method of inducing expression of a heterologous polypeptide in a cell, the method comprising: contacting a cell with a peptide-major histocompatibility complex (peptide-MHC), wherein the cell expresses any of the chimeric polypeptides described above and comprises a sequence encoding the heterologous polypeptide operably linked to a transcriptional control element responsive to a transcriptional activator of the chimeric polypeptide, thereby releasing the intracellular domain of the chimeric polypeptide and inducing expression of the heterologous polypeptide.

In some embodiments, the heterologous polypeptide is a reporter protein, a chimeric antigen receptor (CAR), an antibody, a chimeric bispecific binding member, an engineered T cell receptor (TCR) and an innate-immune response inducer.

Aspects of the instant disclosure include a host cell comprising: a) a nucleic acid encoding any of the chimeric polypeptides described above that specifically binds to a first peptide-major histocompatibility complex (peptide-MHC); and b) a transcriptional control element responsive to a transcriptional activator of the chimeric polypeptide operably linked to a nucleic acid encoding a polypeptide of interest (POI).

In some embodiments, the host cell is genetically modified and the nucleic acid and the transcriptional control element are present within the genome of the host cell. In some embodiments, the nucleic acid and the transcriptional control element are present extrachromosomally within the host cell. In some embodiments, the POI is a heterologous polypeptide. In some embodiments, the heterologous polypeptide is selected from the group consisting of: a reporter protein, a chimeric antigen receptor (CAR), an antibody, a chimeric bispecific binding member, an engineered T cell receptor (TCR) and an innate-immune response inducer. In some embodiments, the heterologous polypeptide is a CAR that specifically binds to a second peptide-MHC. In some embodiments, the specific binding member of the chimeric polypeptide specifically binds to a first peptide-MHC comprising a first intracellular cancer antigen peptide and the CAR specifically binds to a second peptide-MHC comprising a second intracellular cancer antigen peptide. In some embodiments, first intracellular cancer antigen peptide is a WT1 peptide and the second intracellular cancer antigen peptide is a NY-ESO peptide. In some embodiments, the first intracellular cancer antigen peptide is a NY-ESO peptide and the second intracellular cancer antigen peptide is a WT1 peptide. In some embodiments, the heterologous polypeptide is an engineered TCR that specifically binds to a second peptide-MHC. In some embodiments, the specific binding member of the chimeric polypeptide specifically binds to a first peptide-MHC comprising a first intracellular cancer antigen peptide and the engineered TCR specifically binds to a second peptide-MHC comprising a second intracellular cancer antigen peptide. In some embodiments, the first intracellular cancer antigen peptide is a WT1 peptide and the second intracellular cancer antigen peptide is a NY-ESO peptide. In some embodiments, the first intracellular cancer antigen peptide is a NY-ESO peptide and the second intracellular cancer antigen peptide is a WT1 peptide.

Aspects of the instant disclosure include a host cell comprising: a) a nucleic acid encoding a chimeric polypeptide comprising, from N-terminal to C-terminal and in covalent linkage: i) an extracellular domain comprising a specific binding member that specifically binds to a target molecule present on the surface of a cancer cell; ii) a proteolytically cleavable Notch receptor polypeptide comprising one or more proteolytic cleavage sites; and iii) an intracellular domain comprising a transcriptional activator; b) a nucleic acid encoding a chimeric bispecific binding member operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the chimeric bispecific binding member to be expressed.

In some embodiments, the chimeric bispecific binding member comprises a binding domain specific for a cancer antigen and a binding domain specific for a protein expressed on the surface of an immune cell. In some embodiments, the chimeric bispecific binding member comprises at least one antibody derived antigen-binding domains. In some embodiments, the chimeric bispecific binding member is a bispecific antibody or a fragment thereof. In some embodiments, the chimeric bispecific binding member comprises at least one receptor or ligand binding domain of a ligand-receptor binding pair. In some embodiments, the chimeric bispecific binding member comprises at least one antibody derived antigen-binding domain and at least one receptor or ligand binding domain of a ligand-receptor binding pair. In some embodiments, the protein expressed on the surface of an immune cell is CD3. In some embodiments, the protein expressed on the surface of an immune cell is Natural Killer Group 2D (NKG2D) receptor. In some embodiments, the target molecule is a cancer antigen. In some embodiments, the target molecule is a tissue specific molecule. In some embodiments, the target molecule is an organ specific molecule. In some embodiments, the target molecule is a cell type specific molecule.

Aspects of the instant disclosure include a method of treating a subject for a neoplasia comprising administering to the subject an effective amount of host cells according to any of those described above, wherein the neoplasia expresses the target molecule and the cancer antigen.

Aspects of the instant disclosure include a host cell comprising: a) a nucleic acid encoding a chimeric polypeptide comprising, from N-terminal to C-terminal and in covalent linkage: i) an extracellular domain comprising a specific binding member that specifically binds to a target molecule present on the surface of a cancer cell; ii) a proteolytically cleavable Notch receptor polypeptide comprising one or more proteolytic cleavage sites; and iii) an intracellular domain comprising a transcriptional activator; b) a nucleic acid encoding an anti-Fc chimeric antigen receptor (CAR) operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the anti-Fc CAR to be expressed.

In some embodiments, the target molecule is a cancer antigen. In some embodiments, the target molecule is a tissue specific molecule. In some embodiments, the target molecule is an organ specific molecule. In some embodiments, the target molecule is a cell type specific molecule. In some embodiments, the host cell further comprises a nucleic acid encoding an antibody specific for a cancer antigen present on the surface of a cancer cell and comprising an Fc region that is bound by the anti-Fc CAR. In some embodiments, the nucleic acid encoding the antibody is operably linked to the transcriptional control element.

Aspects of the instant disclosure include, a method of treating a subject for a neoplasia comprising administering to the subject an effective amount of any of the host cells described above, wherein the neoplasia expresses the target molecule.

In some embodiments, the method further comprises administering to the subject an antibody specific for a cancer antigen present on the surface of a cancer cell and comprising an Fc region that is bound by the anti-Fc CAR.

Aspects of the instant disclosure include a host cell comprising: a) a nucleic acid encoding a chimeric polypeptide comprising, from N-terminal to C-terminal and in covalent linkage: i) an extracellular domain comprising a specific binding member that specifically binds to a target molecule present on the surface of a cancer cell; ii) a proteolytically cleavable Notch receptor polypeptide comprising one or more proteolytic cleavage sites; and iii) an intracellular domain comprising a transcriptional activator; b) a nucleic acid encoding an innate-immune response inducer operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the innate-immune response inducer to be expressed.

In some embodiments, the target molecule is a tissue specific molecule. In some embodiments, the target molecule is an organ specific molecule. In some embodiments, the target molecule is a cell type specific molecule. In some embodiments, the target molecule is a cancer antigen. In some embodiments, the innate-immune response inducer is bacterial protein or fragment thereof. In some embodiments, the innate-immune response inducer is viral protein or fragment thereof. In some embodiments, the innate-immune response inducer is fungal protein or fragment thereof. In some embodiments, the innate-immune response inducer is a protein or fragment thereof expressed by a mammalian parasite. In some embodiments, the mammalian parasite is a human parasite.

Aspects of the instant disclosure include a method of inducing a local innate immune response in an area of a subject, the method comprising administering to the subject an effective amount of those host cells described above, wherein the area expresses the target molecule. In some embodiments, the area of the subject comprises a neoplasia.

Aspects of the instant disclosure include a host cell comprising: a) a nucleic acid encoding a chimeric polypeptide comprising, from N-terminal to C-terminal and in covalent linkage: i) an extracellular domain comprising a specific binding member that specifically binds to a target molecule present on the surface of a cancer cell; ii) a proteolytically cleavable Notch receptor polypeptide comprising one or more proteolytic cleavage sites; and iii) an intracellular domain comprising a transcriptional activator; b) a nucleic acid encoding an immune suppression factor operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the immune suppression factor to be expressed.

In some embodiments, the target molecule is a tissue specific molecule. In some embodiments, the target molecule is an organ specific molecule. In some embodiments, the target molecule is a cell type specific molecule. In some embodiments, the target molecule is an autoantigen. In some embodiments, the immune suppression factor is an immunosuppressive cytokine. In some embodiments, the immunosuppressive cytokine is IL-10. In some embodiments, the immune suppression factor is a cell-to-cell signaling immunosuppressive ligand. In some embodiments, the cell-to-cell signaling immunosuppressive ligand is PD-L1.

Aspects of the instant disclosure include a method of suppressing an immune response in a subject, the method comprising administering to the subject an effective amount of any of the host cells described above, wherein the subject expresses the target molecule. In some embodiments, the subject has an autoimmune disease.

Aspects of the instant disclosure include a method of killing a heterogeneous tumor, the method comprising: contacting a heterogeneous tumor comprising a first cell expressing a killing antigen and a second cell expressing the killing antigen and a priming antigen with an engineered immune cell comprising: a proteolytically cleavable chimeric polypeptide that specifically binds the priming antigen; a nucleic acid sequence encoding a therapeutic polypeptide that specifically binds the killing antigen; and a transcriptional control element operably linked to the nucleic acid that is responsive to the proteolytically cleavable chimeric polypeptide, wherein binding of the proteolytically cleavable chimeric polypeptide to the priming antigen activates the transcriptional control element to induce expression of the therapeutic polypeptide which, when bound to the killing antigen, kills the first and second cells of the heterogeneous tumor.

In some embodiments, the therapeutic polypeptide is a chimeric antigen receptor (CAR). In some embodiments, the therapeutic polypeptide is a T cell Receptor (TCR). In some embodiments, the therapeutic polypeptide is a therapeutic antibody. In some embodiments, the therapeutic polypeptide is a chimeric bispecific binding member. In some embodiments, at least one of the priming antigen or the killing antigen is an intracellular antigen presented in the context of MHC. In some embodiments, both the priming antigen and the killing antigen are intracellular antigens presented in the context of MHC.

### BRIEF DESCRIPTION OF THE DRAWINGS

FIG. 1 schematically depicts specific binding of an embodiment of a proteolytically cleavable chimeric polypeptide (synNotch) expressed on a receiver cell with a major histocompatibility complex (MHC)-presented antigen of a sender cell to induce expression of a gene or coding sequence (“X”) in the receiver cell.

FIG. 2 demonstrates the specific expression of blue fluorescent protein (BFP) reporter in receiver cells expressing a proteolytically cleavable chimeric polypeptide according to one embodiment described herein, as generally depicted in FIG. 1, specific for MHC-presented WT1 antigen. The reporter is expressed when receiver cells were contacted with sender cells expressing the MHC-presented WT1 antigen but not when contacted with sender cells that do not express the antigen.

FIG. 3 provides quantification related to FIG. 2 showing WT1 intracellular antigen dependent activation of the proteolytically cleavable chimeric polypeptide.

FIG. 4 demonstrates the sensitivity and antigen concentration dependent reporter activation of an anti-MHC-presented WT1 proteolytically cleavable chimeric polypeptide according to one embodiment described herein.

FIG. 5 demonstrates the sensitivity and antigen concentration dependent reporter activation of an anti-MHC-presented WT1 proteolytically cleavable chimeric polypeptide in CD8+ T cells and CD4+ T cells according to one embodiment.

FIG. 6 schematically depicts a proteolytically cleavable chimeric polypeptide-gated circuit utilizing an MHC-presented WT1 specific chimeric polypeptide driving expression of a T cell receptor (TCR) specific for MHC-presented NY-ESO1 antigen. In the depicted embodiment, the T cell becomes activated only when contacted with a target cell expressing both WT1 and NY-ESO1 intracellular antigens.

FIG. 7 depicts proteolytically cleavable chimeric polypeptide-gated T cell activation (as shown by CD69 activation marker expression), according to the system depicted in FIG. 6, only when contacted with target cells expressing both WT1 and NY-ESO1 intracellular antigens.

FIG. 8 depicts significant proteolytically cleavable chimeric polypeptide-gated target cell killing, according to the system depicted in FIG. 6, only when the engineered T cell is contacted with target cells expressing both WT1 and NY-ESO1 intracellular antigens.

FIG. 9 schematically depicts a proteolytically cleavable chimeric polypeptide-gated circuit utilizing an anti-GFP specific chimeric polypeptide driving expression of a T cell receptor (TCR) specific for MHC-presented NY-ESO1 antigen.

FIG. 10 schematically depicts the activation of the T cell depicted in FIG. 9 when contacted with a target expressing both surface-GFP and NY-ESO1 intracellular antigen.

FIG. 11 depicts significant proteolytically cleavable chimeric polypeptide-gated T cell activation (as shown by CD69 activation marker expression), according to the system depicted in FIG. 9 and FIG. 10, only when contacted with target cells expressing both surface-GFP and NY-ESO1 intracellular antigens.

FIG. 12 depicts proteolytically cleavable chimeric polypeptide-gated T cell proliferation, according to the system depicted in FIG. 9 and FIG. 10, when contacted with target cells expressing both surface-GFP and NY-ESO1 intracellular antigens.

FIG. 13 depicts significant proteolytically cleavable chimeric polypeptide-gated T2 target cell killing, according to the system depicted in FIG. 9 and FIG. 10, only when contacted with target cells expressing both surface-GFP and NY-ESO1 intracellular antigens.

FIG. 14 depicts significant proteolytically cleavable chimeric polypeptide-gated A375 target cell killing, according to the system depicted in FIG. 9 and FIG. 10, only when contacted with target cells expressing both surface-GFP and NY-ESO1 intracellular antigens. Comparison with target cell killing by CD8 T cells constitutively expressing an ant-NY-ESO1 TCR is shown to demonstrate enhanced specificity with gated TCR expression.

FIG. 15 provides a general schematic depiction of a dual-intracellular-antigen circuit utilizing an embodiment of a proteolytically cleavable chimeric polypeptide (synNotch) specific for a first intracellular antigen (“antigen A”) to drive expression of a TCR specific for a second intracellular antigen (“antigen B”).

FIG. 16 depicts CD4+ T cells engineered with an α-GFP synNotch receptor controlling the expression of Blinatumomab, an α-CD19/CD3 bi-specific antibody that retargets T cells to CD19+ tumors.

FIG. 17 provides histogram data, related to FIG. 16, showing CD69 (activation marker) expression on the synNotch T cells after co-culture with surface GFP+ only, CD19+ only, or surface GFP/CD19+K562s. The T cells strongly activate in the presence of the surface GFP/CD19 K562s and a small percentage of the T cells activate when incubated with CD19+ only K562s due to low levels of basal leakage of Blinatumomab expression.

FIG. 18 depicts NSG mice subcutaneously injected with CD19-non-target K562s and target CD19+ in the left and right flank, respectively. α-GFP synNotch T cells in control of Blinatumomab (α-CD19/CD3 BiTE) expression were injected i.v. into the mice 4 days after tumor implantation. The tumors were measured by caliper over for 25 days.

FIG. 19 provides the bilateral CD19+ and GFP/CD19+K562 tumor growth curves in mice treated with CD4+ and CD8+ T cells engineered with the α-GFP synNotch receptor controlling Blinatumomab (α-CD19/CD3 BiTE) expression (as depicted in FIG. 18). The dual antigen GFP/CD19+ tumor is selectively cleared (n=5 mice, error=SEM, significance determined by Student's t-test ** p≤0.01).

FIG. 20 depicts CD4+ T cells engineered with the α-GFP synNotch receptor controlling the expression of Flagellin. In the reporter assay, supernatant was harvested from the T cells after co-culture with surface GFP+ or GFP-K562s and added to hTLR5 HEK-blue secreted alkaline phosphatase (SEAP) reporter cells. After 24 hrs. SEAP activity was monitored and the level of Flagellin in the supernatant was measured.

FIG. 21 provides quantification of the induced innate immune mediator reporter assay depicted in FIG. 20.

FIG. 22 schematically depicts programming a T cell to recognize and treat a heterogeneous tumor according to an embodiment described herein.

FIG. 23 schematically depicts a model system designed to demonstrate programming a T cell to recognize and treat a heterogeneous tumor expressing a GFP “priming” antigen and a CD19 “killing” antigen according to an embodiment described herein.

FIG. 24 demonstrates the effective targeting and killing of an in vitro modeled dual-antigen heterogeneous tumor (“heterogeneous mixture”) according to FIG. 23.

FIG. 25 provides further quantification of the model of heterogeneous tumor killing depicted in FIG. 23 using model tumors of various ratios of priming antigen-expressing cells and killing antigen-expressing cells. The “CD19+ target” bars are on top and “GFP/CD19+ target” bars are on bottom.

FIG. 26 provides a time-course of heterogeneous tumor killing according to the model system depicted in FIG. 23 for a tumor having a 1:3 ratio of priming antigen-expressing cells to killing antigen-expressing cells.

FIG. 27 provides a time-course of heterogeneous tumor killing according to the model system depicted in FIG. 23 for a tumor having a 1:19 ratio of priming antigen-expressing cells to killing antigen-expressing cells.

FIG. 28 schematically depicts CD4+ T cells engineered with an α-CD19 synNotch receptor controlling the expression of immunosuppressive agents PD-L1 and IL-10.

FIG. 29 provides quantification of the percentage of synNotch T cells that express PD-L1 and intracellular IL-10 after co-culture with CD19+ or CD19-K562s for 24 hrs as depicted in FIG. 28. The amount of IL-10 in the supernatant was also determined by ELISA.

FIG. 30A-30G provide schematic depictions of exemplary Notch regulatory regions of Notch receptor polypeptides of the present disclosure.

FIG. 31A-31G provide amino acid sequences of Notch receptor polypeptides of various species (SEQ ID NOs: 1-7).

FIG. 32A-32B depict examples of Notch receptor polypeptide Notch regulatory regions and the components therein (SEQ ID NOs: 16-17).

FIG. 33 schematically depicts a proteolytically cleavable chimeric polypeptide-gated circuit driving expression of either a wild-type affinity or an enhanced affinity TCR.

FIG. 34 depicts dual-antigen gated cytokine secretion according to the system depicted in FIG. 33.

FIG. 35 schematically depicts a proteolytically cleavable chimeric polypeptide-gated circuit driving expression of a TCR in an engineered CD4 T cell.

FIG. 36 depicts high level cytokine secretion in response to target cells expressing the targeted dual-antigens according to the system depicted in FIG. 35.

FIG. 37 schematically depicts a proteolytically cleavable chimeric polypeptide-gated circuit targeting a clinically relevant antigen pair using engineered CD4 or CD8 T cells.

FIG. 38 depicts specific killing of target cells expressing the clinically relevant antigen pair by both engineered CD8 and CD4 T cells according to the system depicted in FIG. 37.

FIG. 39 schematically depicts a proteolytically cleavable chimeric polypeptide-gated circuit utilizing an anti-GFP specific chimeric polypeptide driving expression of a TCR specific for HLA-A2/Mart1.

FIG. 40 depicts the specific activation of T cells expressing the circuit schematically depicted in FIG. 39 only in the presence of target cells expressing both the surface GFP and HLA-A2/Mart1 antigens.

FIG. 41 schematically depicts an inside-outside proteolytically cleavable chimeric polypeptide-gated circuit utilizing an anti-HLA-A2/WT1 specific chimeric polypeptide driving expression of an anti-HER2 CAR.

FIG. 42 depicts the specific activation of T cells expressing the circuit schematically depicted in FIG. 41 only in the presence of target cells expressing both the inside antigen (HLA-A2/WT1) and the outside antigen (HER2).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242:423-426 (1988)), regarding single chain antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and—binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. In some cases, a specific binding member present in the extracellular domain of a chimeric polypeptide of the present disclosure binds specifically to a peptide-major histocompatibility complex (peptide-MHC). “Specific binding” refers to binding with an affinity of at least about 10⁻⁷ M or greater, e.g., 5×10⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215): 215ra172; Glienke et al. Front Pharmacol (2015) 6:21; Kakarla & Gottschalk 52 Cancer J (2014) 20(2): 151-5; Riddell et al. Cancer J (2014) 20(2): 141-4; Pegram et al. Cancer J (2014) 20(2): 127-33; Cheadle et al. Immunol Rev (2014) 257(1): 91-106; Barrett et al. Annu Rev Med (2014) 65:333-47; Sadelain et al. Cancer Discov (2013) 3(4): 388-98; Cartellieri et al., J Biomed Biotechnol (2010) 956304; the disclosures of which are incorporated herein by reference in their entirety. Useful CARs also include the anti-CD19-4-1BB-CD3ζ CAR expressed by lentivirus loaded CTL019 (Tisagenlecleucel-T) CAR-T cells as commercialized by Novartis (Basel, Switzerland).

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4+ cells), cytotoxic T-cells (CD8+ cells), T-regulatory cells (Treg) and gamma-delta T cells.

A “cytotoxic cell” includes CD8+ T cells, natural-killer (NK) cells, and neutrophils, which cells are capable of mediating cytotoxicity responses.

The term “synthetic” as used herein generally refers to an artificially derived polypeptide or polypeptide encoding nucleic acid that is not naturally occurring. Such synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The instant disclosure provides chimeric polypeptides which modulate various cellular processes following a cleavage event induced upon binding of a specific binding member of the chimeric polypeptide with its binding partner. Methods of using chimeric polypeptides to modulate cellular functions, including e.g., induction of gene expression, are also provided. Nucleic acids encoding the subject chimeric polypeptides and associated expression cassettes and vectors as well as cells that contain such nucleic acids and/or expression cassettes and vectors are provided. Also provided, are methods of treating a subject using the described components and methods as well as kits for practicing the subject methods.

### Methods

Methods are provided for modulating one or more cellular processes and/or activities and/or functions using chimeric polypeptides that undergo a binding-induced cleavage event to release an intracellular domain from the chimeric polypeptide. As described in more detail below, chimeric polypeptides of the instant disclosure may generally include: a) an extracellular domain comprising a specific binding member; b) a proteolytically cleavable Notch receptor polypeptide comprising one or more proteolytic cleavage sites; and c) an intracellular domain. Methods of the instant disclosure include using such chimeric polypeptides to modulate one or more cellular processes and/or activities and/or functions upon binding of the specific binding member to its binding partner.

According to the methods described herein, in some instances, chimeric polypeptides are expressed from a nucleic acid, within or introduced into a cell, which encodes the chimeric polypeptide. As such, in some instances, the instant methods may include contacting a cell with a nucleic acid encoding a chimeric polypeptide wherein such contacting is sufficient to introduce the nucleic acid into the cell. Any convenient method of introducing nucleic acids into a cell may find use herein including but not limited viral transfection, electroporation, lipofection, bombardment, chemical transformation, use of a transducible carrier (e.g., a transducible carrier protein), and the like.

Introduced nucleic acids may be maintained within the cell or transiently present. As such, in some instance, an introduced nucleic acid may be maintained within the cell, e.g., integrated into the genome. Any convenient method of nucleic acid integration may find use in the subject methods, including but not limited to e.g., viral-based integration, transposon-based integration, homologous recombination-based integration, and the like. In some instance, an introduced nucleic acid may be transiently present, e.g., extrachromosomally present within the cell. Transiently present nucleic acids may persist, e.g., as part of any convenient transiently transfected vector.

An introduced nucleic acid encoding a chimeric polypeptide of the instant disclosure may be introduced in such a manner as to be operably linked to a promoter that drives the expression of the chimeric polypeptide. The source of such promoters may vary and may include e.g., where the promoter is introduced with the nucleic acid, e.g., as part of an expression construct or where the promoter is present in the cell prior to introducing the nucleic acid or introduced after the nucleic acid. As described in more detail herein, useful promoters can include endogenous promoters and heterologous promoters. For example, in some instances, a nucleic acid may be introduced as part of an expression construct containing a heterologous promoter operably linked to the nucleic acid. In some instances, a nucleic acid may be introduced as part of an expression construct containing a copy of a promoter that is endogenous to the cell into which the nucleic acid is introduced. In some instances, a nucleic acid may be introduced without a promoter and, upon integration into the genome of the cell, the nucleic acid may be operably linked to an endogenous promoter already present in the cell. Depending on the confirmation and/or the promoter utilized, expression of the chimeric polypeptide from the nucleic acid may be configured to be constitutive, inducible, tissue-specific, cell-type specific, etc., including combinations thereof.

Chimeric polypeptides of the instant disclosure within a cell, regardless of the method of introduction, generally will reside in the plasma membrane and remain inactive when the specific binding member of such a chimeric polypeptide is not bound by its binding partner. As used herein, in relationship to chimeric polypeptides of the instant disclosure, by “inactive” is meant the intracellular domain of the chimeric polypeptide remains linked to the cleavable polypeptide (e.g., cleavable Notch polypeptide) such that the intracellular domain is sequestered and unable to modulate intracellular functions and/or cellular activities. Upon binding of the specific binding member to its binding partner the chimeric polypeptide may be said to become active, wherein the term “active” generally refers to the release of the intracellular domain from the chimeric polypeptide by a cleavage event triggered by the binding, such that the intracellular domain is freed and may influence intracellular functions and/or cellular activities.

Cellular processes and/or activities and/or functions that may be modulated according to the instant methods will vary any may include but are not limited to modulating expression of a gene or other coding sequence, e.g., inducing expression of a gene or coding sequence, repressing expression of a gene or coding sequence, etc. Accordingly, in some instances, the intracellular domain of a chimeric polypeptide used in the subject methods may include a transcriptional modulator, including e.g., a transcriptional activator or a transcriptional repressor.

In some instances, cellular processes and/or activities and/or functions that may be modulated include but are not limited to e.g., expression of a gene product of the cell, proliferation of the cell, apoptosis of the cell, non-apoptotic death of the cell, differentiation of the cell, dedifferentiation of the cell, migration of the cell, secretion of a molecule from the cell (e.g., secretion of a therapeutic polypeptide, secretion of a cytokine, etc.), cellular adhesion of the cell, immune cell activation (e.g., T cell activation, etc.), production of effector molecules (e.g., cytokines, antibodies, growth factors, etc.), transcription of a target nucleic acid, translation of a target mRNA, organelle activity, intracellular trafficking, and the like.

In some instances, the expression and/or secretion of a cytokine may be modulated. Non-limiting examples of cytokines, the expression/secretion of which may be modulated, include but are not limited to e.g., Interleukins and related (e.g., IL-1-like, IL-1a, IL-1B, IL-1RA, IL-18, IL-2, IL-4, IL-7, IL-9, IL-13, IL-15, IL-3, IL-5, GM-CSF, IL-6-like, IL-6, IL-11, G-CSF, IL-12, LIF, OSM, IL-10-like, IL-10, IL-20, IL-14, IL-16, IL-17, etc.), Interferons (e.g., IFN-α, IFN-β, IFN-γ, etc.), TNF family (e.g., CD154, LT-β, TNF-α, TNF-β, 4-1BBL, APRIL, CD70, CD153, CD178, GITRL, LIGHT, OX40L, TALL-1, TRAIL, TWEAK, TRANCE, etc.), TGF-β family (e.g., TGF-β1, TGF-β2, TGF-β3, etc.) and the like. In some instances, activation of a cell through a chimeric polypeptide of the present disclosure, or a plurality thereof, may induce an increase in cytokine expression and/or secretion relative to that of a comparable cell where the chimeric polypeptide is not present or otherwise inactive. The amount of the increase may vary and may range from a 10% or greater increase, including but not limited to e.g., 10% or greater, 25% or greater, 50% or greater, 75% or greater, 100% or greater, 150% or greater, 200% or greater, 250% or greater, 300% or greater, 350% or greater 400% or greater, etc.

In some instances, where dual-antigen recognition is employed, described in more detail below, a cell activated by binding of both antigens of a target cell may display an increase in cellular activation or expression/secretion of a cytokine as compared to a corresponding cell bound to only one antigen. In some instance, such an increase may range from a 10% or greater increase, including but not limited to e.g., 10% or greater, 25% or greater, 50% or greater, 75% or greater, 100% or greater, 150% or greater, 200% or greater, 250% or greater, 300% or greater, 350% or greater 400% or greater, etc.

In some instances, the methods described herein include methods of inducing expression of a polypeptide in a cell expressing a chimeric polypeptide of the instant disclosure by contacting the cell with a binding partner of the specific binding member of the chimeric polypeptide. Depending on the particular configuration, such methods may include inducing expression of an endogenous gene or coding sequence or a heterologous gene or coding sequence. In some instances, the binding partner of the specific binding member may be present on the surface of a cell. In some instances, the binding partner of the specific binding member may not be present on the surface of a cell and may be e.g., bound to a substrate (e.g., a solid support such as the surface of a plate or bead), unbound or freely diffusible, etc. Accordingly, where methods described herein include contacting a cell with a binding partner of a specific binding member of a chimeric polypeptide, such contacting may include but is not limited to e.g., contacting with medium containing freely diffusible binding partner, contacting with cells expressing the binding partner on their surface, contacting with a substrate with attached binding partner, etc. Unbound or freely diffusible specific binding members may, in some instances, function as a soluble adapter molecule, e.g., facilitating binding between an anchor cell and a receiver cell to generate the force necessary to activate a subject cleavable chimeric polypeptide, e.g., as described in PCT Application No. US2016/019188 (Pub. No. WO 2016/138034); the disclosure of which is incorporated herein by reference in its entirety. In some instances, an unbound or freely diffusible binding partner may be subsequently captured or anchored by any other convenient means, including but not limited to e.g., the introduction of an additional binding partner that specifically binds the unbound or freely diffusible binding partner and is bound to or otherwise associated with a substrate or the surface of a cell.

In the subject methods, any convenient pair of specific binding member and binding partner may be utilized, provided the pair specifically binds to one another sufficiently to activate the chimeric polypeptide. In some instances, a useful pair of specific binding member and binding partner may include an antigen-antibody pair, where e.g., the antibody is utilized as the specific binding member and the antigen as the binding partner or the antigen is utilized as the specific binding member and the antibody as the binding partner.

In some instances, the methods described herein include methods of modulating a cellular activity of a cell expressing a chimeric polypeptide by contacting the cell with a peptide-major histocompatibility complex (peptide-MHC) under conditions sufficient for the peptide-MHC to bind the specific binding member of the chimeric polypeptide. In some instances, the binding of the peptide-MHC to the chimeric polypeptide activates the chimeric polypeptide releasing the intracellular domain and inducing expression of a polypeptide within the cell.

Where methods of the instant disclosure include contacting a cell expressing a chimeric polypeptide with a binding partner to induce expression of a gene or coding sequence, essentially any polypeptide, natural or recombinant, may be induced to be expressed. In some instances, an expressed polypeptide may be referred to as a polypeptide of interest (POI). A POI may be essentially any polypeptide and may include but is not limited to polypeptides of research interest (e.g., reporter polypeptides, mutated polypeptides, novel synthetic polypeptides, etc.), polypeptides of therapeutic interest (e.g., naturally occurring therapeutic proteins, recombinant therapeutic polypeptides, etc.), polypeptides of industrial interest (e.g., polypeptides used in industrial applications such as e.g., manufacturing), and the like.

In some instances, polypeptides induced to be expressed may include but are not limited to e.g., reporter proteins, chimeric antigen receptors (CAR), antibodies, chimeric bispecific binding members, engineered T cell receptors (TCR), innate-immune response inducers, etc.

“Contacting” of the instant methods may vary depending on the context and may include in vitro contacting, ex vivo contacting, and in vivo contacting. For example, in some instances, e.g., where a cell expressing a chimeric polypeptide is cultured in vitro, the contacting may include adding the binding partner or a cell expressing the binding partner or a substrate, with the binding partner attached, to the in vitro culture. In some instances, e.g., where the binding partner is present in an individual in vivo, including e.g., present on a cell present in the individual in vivo, the contacting may include administering a cell expressing the chimeric polypeptide to the individual. In some instances, e.g., where the cell expressing the chimeric polypeptide is present in an individual in vivo the contacting may include administering the binding partner to the individual, removing the cell from the individual and contacting the cell with the binding partner ex vivo, causing or allowing both the chimeric polypeptide and the binding partner to be simultaneously expressed in vivo, etc.

Methods of the present disclosure for modulating the activity of a cell can be carried out in a single cell, or in a multicellular environment (e.g., a naturally-occurring tissue; an artificial tissue; etc.). Methods of the present disclosure for modulating the activity of a cell can be carried out in parallel or in series.

Methods of the instant disclosure may further include culturing a cell expressing a chimeric polypeptide of the instant disclosure including but not limited to e.g., culturing the cell prior to contacting the cell with the binding partner, culturing the cell while contacting the cell with the binding partner, culturing the cell following contacting the cell with the binding partner. Any convenient method of cell culture may be employed whereas such methods will vary based on various factors including but not limited to e.g., the type of cell being cultured, the intended use of the cell (e.g., whether the cell is cultured for research or therapeutic purposes), etc. In some instances, methods of the instant disclosure may further include common processes of cell culture including but not limited to e.g., seeding cell cultures, feeding cell cultures, passaging cell cultures, splitting cell cultures, analyzing cell cultures, treating cell cultures with a drug, harvesting cell cultures, etc.

Methods of the instant disclosure may, in some instances, further include receiving and/or collecting cells that are used in the subject methods. In some instances, cells are collected from a subject. Collecting cells from a subject may include obtaining a tissue sample from the subject and enriching, isolating and/or propagating the cells from the tissue sample. Isolation and/or enrichment of cells may be performed using any convenient method including e.g., isolation/enrichment by culture (e.g., adherent culture, suspension culture, etc.), cell sorting (e.g., FACS), and the like. Cells may be collected from any convenient cellular tissue sample including but not limited to e.g., blood (including e.g., peripheral blood, cord blood, etc.), bone marrow, a biopsy, a skin sample, a cheek swab, etc. In some instances, cells are received from a source including e.g., a blood bank, tissue bank, etc. Received cells may have been previously isolated or may be received as part of a tissue sample thus isolation/enrichment may be performed after receiving the cells and prior to use. In certain instances, received cells may be non-primary cells including e.g., cells of a cultured cell line. Suitable cells for use in the herein described methods are further detailed herein.

### Methods of Treatment

Methods of the present disclosure include methods of treating a subject using one or more proteolytically cleavable chimeric polypeptides as described herein. Any convenient method of delivering the chimeric polypeptide may find use in the subject methods. In some instances, the subject chimeric polypeptides may be delivered by administering to the subject a cell expressing the chimeric polypeptide. In some instances, the subject chimeric polypeptides may be delivered by administering to the subject a nucleic acid comprising a nucleotide sequence encoding the chimeric polypeptide. Administering to a subject a nucleic acid encoding the chimeric polypeptide may include administering to the subject a cell containing the nucleic acid where the nucleic acid may or may not yet be expressed. In some instances, administering to a subject a nucleic acid encoding the chimeric polypeptide may include administering to the subject a vector designed to deliver the nucleic acid to a cell.

Accordingly, in the subject methods of treatment, nucleic acids encoding chimeric polypeptides may be administered in vitro, ex vivo or in vivo. In some instances, cells may be collected from a subject and transfected with nucleic acid and the transfected cells may be administered to the subject, with or without further manipulation including but not limited to e.g., in vitro expansion. In some instances, the nucleic acid, e.g., with or without a delivery vector, may be administered directly to the subject.

Given the diversity of cellular activities that may be modulated through the use of the subject proteolytically cleavable chimeric polypeptides, the instant methods of treatment may be utilized for a variety of applications. As non-limiting examples, the instant methods may find use in a treatment directed to a variety of diseases including but not limited to e.g., Acanthamoeba infection, Acinetobacter infection, Adenovirus infection, ADHD (Attention Deficit/Hyperactivity Disorder), AIDS (Acquired Immune Deficiency Syndrome), ALS (Amyotrophic Lateral Sclerosis), Alzheimer's Disease, Amebiasis, Intestinal (Entamoeba histolytica infection), Anaplasmosis, Human, Anemia, Angiostrongylus Infection, Animal-Related Diseases, Anisakis Infection (Anisakiasis), Anthrax, Aortic Aneurysm, Aortic Dissection, Arenavirus Infection, Arthritis (e.g., Childhood Arthritis, Fibromyalgia, Gout, Lupus (SLE) (Systemic lupus erythematosus), Osteoarthritis, Rheumatoid Arthritis, etc.), Ascaris Infection (Ascariasis), Aspergillus Infection (Aspergillosis), Asthma, Attention Deficit/Hyperactivity Disorder, Autism, Avian Influenza, B virus Infection (Herpes B virus), B. cepacia infection (Burkholderia cepacia Infection), Babesiosis (Babesia Infection), Bacterial Meningitis, Bacterial Vaginosis (BV), Balamuthia infection (Balamuthia mandrillaris infection), Balamuthia mandrillaris infection, Balantidiasis, Balantidium Infection (Balantidiasis), Baylisascaris Infection, Bilharzia, Birth Defects, Black Lung (Coal Workers' Pneumoconioses), Blastocystis hominis Infection, Blastocystis Infection, Blastomycosis, Bleeding Disorders, Blood Disorders, Body Lice (Pediculus humanus corporis), Borrelia burgdorferi Infection, Botulism (Clostridium botulinim), Bovine Spongiform Encephalopathy (BSE), Brainerd Diarrhea, Breast Cancer, Bronchiolitis, Bronchitis, Brucella Infection (Brucellosis), Brucellosis, Burkholderia cepacia Infection (B. cepacia infection), Burkholderia mallei, Burkholderia pseudomallei Infection, Campylobacter Infection (Campylobacteriosis), Campylobacteriosis, Cancer (e.g., Colorectal (Colon) Cancer, Gynecologic Cancers, Lung Cancer, Prostate Cancer, Skin Cancer, etc.), Candida Infection (Candidiasis), Candidiasis, Canine Flu, Capillaria Infection (Capillariasis), Capillariasis, Carbapenem resistant Klebsiella pneumonia (CRKP), Cat Flea Tapeworm, Cercarial Dermatitis, Cerebral Palsy, Cervical Cancer, Chagas Disease (Trypanosoma cruzi Infection), Chickenpox (Varicella Disease), Chikungunya Fever (CHIKV), Childhood Arthritis, German Measles (Rubella Virus), Measles, Mumps, Rotavirus Infection, Chlamydia (Chlamydia trachomatis Disease), Chlamydia pneumoniae Infection, Chlamydia trachomatis Disease, Cholera (Vibrio cholerae Infection), Chronic Fatigue Syndrome (CFS), Chronic Obstructive Pulmonary Disease (COPD), Ciguatera Fish Poisoning, Ciguatoxin, Classic Creutzfeldt-Jakob Disease, Clonorchiasis, Clonorchis Infection (Clonorchiasis), Clostridium botulinim, Clostridium difficile Infection, Clostridium perfringens infection, Clostridium tetani Infection, Clotting Disorders, CMV (Cytomegalovirus Infection), Coal Workers' Pneumoconioses, Coccidioidomycosis, Colorectal (Colon) Cancer, Common Cold, Conjunctivitis, Cooleys Anemia, COPD (Chronic Obstructive Pulmonary Disease), Corynebacterium diphtheriae Infection, Coxiella burnetii Infection, Creutzfeldt-Jakob Disease, CRKP (Carbapenem resistant Klebsiella pneumonia), Crohn's Disease, Cryptococcosis, Cryptosporidiosis, Cryptosporidium Infection (Cryptosporidiosis), Cyclospora Infection (Cyclosporiasis), Cyclosporiasis, Cysticercosis, Cystoisospora Infection (Cystoisosporaiasis), Cystoisosporaiasis, Cytomegalovirus Infection (CMV), Dengue Fever (DF), Dengue Hemorrhagic Fever (DHF), Dermatophytes, Dermopathy, Diabetes, Diamond Blackfan Anemia (DBA), Dientamoeba fragilis Infection, Diphtheria (Corynebacterium diphtheriae Infection), Diphyllobothriasis, Diphyllobothrium Infection (Diphyllobothriasis), Dipylidium Infection, Dog Flea Tapeworm, Down Syndrome (Trisomy 21), Dracunculiasis, Dwarf Tapeworm (Hymenolepis Infection), E. coli Infection (Escherichia coli Infection), Ear Infection (Otitis Media), Eastern Equine Encephalitis (EEE), Ebola Hemorrhagic Fever, Echinococcosis, Ehrlichiosis, Elephantiasis, Encephalitis (Mosquito-Borne and Tick-Borne), Entamoeba histolytica infection, Enterobius vermicularis Infection, Enterovirus Infections (Non-Polio), Epidemic Typhus, Epilepsy, Epstein-Barr Virus Infection (EBV Infection), Escherichia coli Infection, Extensively Drug-Resistant TB (XDR TB), Fasciola Infection (Fascioliasis), Fasciolopsis Infection (Fasciolopsiasis), Fibromyalgia, Fifth Disease (Parvovirus B19 Infection), Flavorings-Related Lung Disease, Folliculitis, Food-Related Diseases, Clostridium perfringens infection, Fragile X Syndrome, Francisella tularensis Infection, Genital Candidiasis (Vulvovaginal Candidiasis (VVC)), Genital Herpes (Herpes Simplex Virus Infection), Genital Warts, German Measles (Rubella Virus), Giardia Infection (Giardiasis), Glanders (Burkholderia mallei), Gnathostoma Infection, Gnathostomiasis (Gnathostoma Infection), Gonorrhea (Neisseria gonorrhoeae Infection), Gout, Granulomatous amebic encephalitis (GAE), Group A Strep Infection (GAS) (Group A Streptococcal Infection), Group B Strep Infection (GBS) (Group B Streptococcal Infection), Guinea Worm Disease (Dracunculiasis), Gynecologic Cancers (e.g., Cervical Cancer, Ovarian Cancer, Uterine Cancer, Vaginal and Vulvar Cancers, etc.), H1N1 Flu, Haemophilus influenzae Infection (Hib Infection), Hand, Foot, and Mouth Disease (HFMD), Hansen's Disease, Hantavirus Pulmonary Syndrome (HPS), Head Lice (Pediculus humanus capitis), Heart Disease (Cardiovascular Health), Heat Stress, Hemochromatosis, Hemophilia, Hendra Virus Infection, Herpes B virus, Herpes Simplex Virus Infection, Heterophyes Infection (Heterophyiasis), Hib Infection (Haemophilus influenzae Infection), High Blood Pressure, Histoplasma capsulatum Disease, Histoplasmosis (Histoplasma capsulatum Disease), Hot Tub Rash (Pseudomonas dermatitis Infection), HPV Infection (Human Papillomavirus Infection), Human Ehrlichiosis, Human Immunodeficiency Virus, Human Papillomavirus Infection (HPV Infection), Hymenolepis Infection, Hypertension, Hyperthermia, Hypothermia, Impetigo, Infectious Mononucleosis, Inflammatory Bowel Disease (IBD), Influenza, Avian Influenza, H1N1 Flu, Pandemic Flu, Seasonal Flu, Swine Influenza, Invasive Candidiasis, Iron Overload (Hemochromatosis), Isospora Infection (Isosporiasis), Japanese Encephalitis, Jaundice, K. pneumoniae (Klebsiella pneumoniae), Kala-Azar, Kawasaki Syndrome (KS), Kernicterus, Klebsiella pneumoniae (K. pneumoniae), La Crosse Encephalitis (LAC), La Crosse Encephalitis virus (LACV), Lassa Fever, Latex Allergies, Lead Poisoning, Legionnaires' Disease (Legionellosis), Leishmania Infection (Leishmaniasis), Leprosy, Leptospira Infection (Leptospirosis), Leptospirosis, Leukemia, Lice, Listeria Infection (Listeriosis), Listeriosis, Liver Disease and Hepatitis, Loa loa Infection, Lockjaw, Lou Gehrig's Disease, Lung Cancer, Lupus (SLE) (Systemic lupus erythematosus), Lyme Disease (Borrelia burgdorferi Infection), Lymphatic Filariasis, Lymphedema, Lymphocytic Choriomeningitis (LCMV), Lymphogranuloma venereum Infection (LGV), Malaria, Marburg Hemorrhagic Fever, Measles, Melioidosis (Burkholderia pseudomallei Infection), Meningitis (Meningococcal Disease), Meningococcal Disease, Methicillin Resistant Staphylococcus aureus (MRSA), Micronutrient Malnutrition, Microsporidia Infection, Molluscum Contagiosum, Monkey B virus, Monkeypox, Morgellons, Mosquito-Borne Diseases, Mucormycosis, Multidrug-Resistant TB (MDR TB), Mumps, Mycobacterium abscessus Infection, Mycobacterium avium Complex (MAC), Mycoplasma pneumoniae Infection, Myiasis, Naegleria Infection (Primary Amebic Meningoencephalitis (PAM)), Necrotizing Fasciitis, Neglected Tropical Diseases (NTD), Neisseria gonorrhoeae Infection, Neurocysticercosis, New Variant Creutzfeldt-Jakob Disease, Newborn Jaundice (Kernicterus), Nipah Virus Encephalitis, Nocardiosis, Non-Polio Enterovirus Infections, Nonpathogenic (Harmless) Intestinal Protozoa, Norovirus Infection, Norwalk-like Viruses (NLV), Novel H1N1 Flu, Onchocerciasis, Opisthorchis Infection, Oral Cancer, Orf Virus, Oropharyngeal Candidiasis (OPC), Osteoarthritis (OA), Osteoporosis, Otitis Media, Ovarian Cancer, Pandemic Flu, Paragonimiasis, Paragonimus Infection (Paragonimiasis), Parasitic Diseases, Parvovirus B19 Infection, Pediculus humanus capitis, Pediculus humanus corporis, Pelvic Inflammatory Disease (PID), Peripheral Arterial Disease (PAD), Pertussis, Phthiriasis, Pink Eye (Conjunctivitis), Pinworm Infection (Enterobius vermicularis Infection), Plague (Yersinia pestis Infection), Pneumocystis jirovecii Pneumonia, Pneumonia, Polio Infection (Poliomyelitis Infection), Pontiac Fever, Prion Diseases (Transmissible spongiform encephalopathies (TSEs)), Prostate Cancer, Pseudomonas dermatitis Infection, Psittacosis, Pubic Lice (Phthiriasis), Pulmonary Hypertension, Q Fever (Coxiella burnetii Infection), Rabies, Raccoon Roundworm Infection (Baylisascaris Infection), Rat-Bite Fever (RBF) (Streptobacillus moniliformis Infection), Recreational Water Illness (RWI), Relapsing Fever, Respiratory Syncytial Virus Infection (RSV), Rheumatoid Arthritis (RA), Rickettsia rickettsii Infection, Rift Valley Fever (RVF), Ringworm (Dermatophytes), Ringworm in Animals, River Blindness (Onchocerciasis), Rocky Mountain Spotted Fever (RMSF) (Rickettsia rickettsii Infection), Rotavirus Infection, RVF (Rift Valley Fever), RWI (Recreational Water Illness), Salmonella Infection (Salmonellosis), Scabies, Scarlet Fever, Schistosomiasis (Schistosoma Infection), Seasonal Flu, Severe Acute Respiratory Syndrome, Sexually Transmitted Diseases (STDs) (e.g., Bacterial Vaginosis (BV), Chlamydia, Genital Herpes, Gonorrhea, Human Papillomavirus Infection, Pelvic Inflammatory Disease, Syphilis, Trichomoniasis, HIV/AIDS, etc.), Shigella Infection (Shigellosis), Shingles (Varicella Zoster Virus (VZV)), Sickle Cell Disease, Single Gene Disorders, Sinus Infection (Sinusitus), Skin Cancer, Sleeping Sickness (African Trypanosomiasis), Smallpox (Variola Major and Variola Minor), Sore Mouth Infection (Orf Virus), Southern Tick-Associated Rash Illness (STARI), Spina Bifida (Myelomeningocele), Sporotrichosis, Spotted Fever Group Rickettsia (SFGR), St. Louis Encephalitis, Staphylococcus aureus Infection, Streptobacillus moniliformis Infection, Streptococcal Diseases, Streptococcus pneumoniae Infection, Stroke, Strongyloides Infection (Strongyloidiasis), Sudden Infant Death Syndrome (SIDS), Swimmer's Itch (Cercarial Dermatitis), Swine Influenza, Syphilis (Treponema pallidum Infection), Systemic lupus erythematosus, Tapeworm Infection (Taenia Infection), Testicular Cancer, Tetanus Disease (Clostridium tetani Infection), Thrush (Oropharyngeal Candidiasis (OPC)), Tick-borne Relapsing Fever, Tickborne Diseases (e.g., Anaplasmosis, Babesiosis, Ehrlichiosis, Lyme Disease, Tourette Syndrome (TS), Toxic Shock Syndrome (TSS), Toxocariasis (Toxocara Infection), Toxoplasmosis (Toxoplasma Infection), Trachoma Infection, Transmissible spongiform encephalopathies (TSEs), Traumatic Brain Injury (TBI), Trichinellosis (Trichinosis), Trichomoniasis (Trichomonas Infection), Tuberculosis (TB) (Mycobacterium tuberculosis Infection), Tularemia (Francisella tularensis Infection), Typhoid Fever (Salmonella typhi Infection), Uterine Cancer, Vaginal and Vulvar Cancers, Vancomycin-Intermediate/Resistant Staphylococcus aureus Infections (VISA/VRSA), Vancomycin-resistant Enterococci Infection (VRE), Variant Creutzfeldt-Jakob Disease (vCJD), Varicella-Zoster Virus Infection, Variola Major and Variola Minor, Vibrio cholerae Infection, Vibrio parahaemolyticus Infection, Vibrio vulnificus Infection, Viral Gastroenteritis, Viral Hemorrhagic Fevers (VHF), Viral Hepatitis, Viral Meningitis (Aseptic Meningitis), Von Willebrand Disease, Vulvovaginal Candidiasis (VVC), West Nile Virus Infection, Western Equine Encephalitis Infection, Whipworm Infection (Trichuriasis), Whitmore's Disease, Whooping Cough, Xenotropic Murine Leukemia Virus-related Virus Infection, Yellow Fever, Yersinia pestis Infection, Yersiniosis (Yersinia enterocolitica Infection), Zoonotic Hookworm, Zygomycosis, and the like.

In some instances, methods of treatment utilizing one or more proteolytically cleavable polypeptides of the instant disclosure may find use in treating a cancer. Cancers, the treatment of which may include the use of one or more proteolytically cleavable polypeptides of the instant disclosure, will vary and may include but are not limited to e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenstrom, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

In some instances, a method of the instant disclosure will include treating a neoplasia by administering to a subject having the neoplasia a cell expressing a chimeric polypeptide of the instant disclosure or a nucleic acid encoding a chimeric polypeptide of the instant disclosure. In some instances, such a method may further include administering to the subject a nucleic acid operably linked to a transcriptional control element that is regulated by the intracellular domain of the chimeric polypeptide. As used herein, the term “neoplasia” generally refers to an abnormal growth of tissue or an abnormally proliferating cell or population of cells, including but not limited to solid tumors, blood cancers, etc., including e.g., those of any cancer, including e.g., those cancers listed herein. A neoplasia may be benign or malignant.

In some instances, the instant methods may be applied to the treatment of heterogeneous tumors. As used herein, the term “heterogeneous tumors” generally refers to a tumor having at least two different types of tumor cells differentially expressing at least one antigen. For example, a heterogeneous tumor may include one type of tumor cell expressing a first antigen and a second type of tumor cell that does not express the antigen. In some instances, a heterogeneous tumor may include one type of tumor cell highly expressing a first antigen and a second type of tumor cell having low expression of the antigen. By “low expression” is meant that the antigen is expressed at a level that makes directly targeting the antigen with a therapeutic impractical. Methods of targeting a heterogeneous tumor as described herein will generally include therapeutically targeting at least two different cell types of the tumor, including e.g., two cell types that differentially express an antigen. Accordingly, the herein described method of targeting a heterogeneous tumor may allow for a therapeutic effect on a cell type of the tumor that does not express or shows low expression of an antigen of a cell type targeted in the method.

Differentially expressed antigens useful in the described methods of treating a heterogeneous tumor may essentially include any antigen that may be targeted with a specific binding member as described herein, including but not limited to e.g., cancer cell antigens (e.g., surface expressed cancer antigens, intracellular cancer antigens, etc.), tissue specific antigens, cell type specific antigens, and the like. In some instances, antigens are endogenously expressed by the cell. In some instances, an antigen may be heterologous to the cell from which it is expressed, including e.g., where an expressed heterologous protein serves as an antigen.

In some instances, a method of treating a heterogeneous tumor may include contacting the tumor with an immune cell engineered to express a proteolytically cleavable chimeric polypeptide specific for a priming antigen. As used herein, the term “priming antigen” generally refers to an antigen sufficient to activate the chimeric polypeptide in the proximity of the heterogeneous tumor. In some instances, a priming antigen may be an antigen present in a subset of cells of the heterogeneous tumor, e.g., present on some cells of the heterogeneous tumor but not present in all cells of the heterogeneous tumor. In some instances, upon activation of a chimeric polypeptide by a priming antigen the freed intracellular domain of the chimeric polypeptide may induce expression of a second antigen-specific polypeptide. In some instances, the antigen of the second antigen-specific polypeptide may be referred to herein as a “therapeutic antigen” or a “killing antigen”. As used herein, the term “therapeutic antigen” may generally refer to the antigen to which a therapeutic construct is directed, e.g., an antigen that is directly targeted by a therapeutic construct including but not limited to e.g., an antibody, a CAR, a TCR, a chimeric bispecific binding member, and the like. As used herein, the term “killing antigen” may generally refer to the antigen to which a construct designed to target a cell for killing is directed, e.g., an antigen that is targeted by a construct that results in killing of the cell expressing the killing antigen by an immune cell including but not limited to e.g., an antibody, a CAR, a TCR, a chimeric bispecific binding member, and the like. In some instances, the second antigen-specific polypeptide may be directed to a therapeutic antigen that is present in all or nearly all or most cells of the heterogeneous tumor.

In some instances, the methods described herein include inducing an innate immune response in a subject. In some instances, a chimeric polypeptide of the instant disclosure may induce the expression of a polypeptide that, when expressed, induces an innate immune response in a subject. As the specific binding member of a chimeric polypeptide of the instant disclosure may be engineered to activate the chimeric polypeptide in response to binding a specific antigen, in some instances, an innate immune response may be induced in response to the presence of a particular antigen. A chimeric polypeptide may be engineered to be activated by any convenient and appropriate antigen including but not limited to e.g., a cancer antigen, a cell type specific antigen, a tissue specific antigen, an infectious disease antigen (e.g., a bacterial antigen, a viral antigen, a fungal antigen, a pathogenic antigen, etc.), and the like. In some instances, the innate immune response may be locally activated e.g., based on the local presence of the antigen, e.g., an antigen locally present in a tumor, an antigen locally present in the tumor microenvironment, an antigen locally present in an infected area or tissue, etc.

In some instances, the methods described herein include controlling expression of one or more immune suppression factors in a subject. In some instances, a chimeric polypeptide of the instant disclosure may induce the expression of a polypeptide that, when expressed, induces immune suppression in a subject. As the specific binding member of a chimeric polypeptide of the instant disclosure may be engineered to activate the chimeric polypeptide in response to binding a specific antigen, in some instances, an immunosuppressive response may be induced in response to the presence of a particular antigen. A chimeric polypeptide may be engineered to be activated by any convenient and appropriate antigen including but not limited to e.g., an autoantigen (e.g., a self-antigen that induces an autoimmune response), a cell type specific antigen, a tissue specific antigen, and the like. In some instances, the immunosuppression may be locally activated e.g., based on the local presence of the antigen, e.g., an antigen locally present in a tissue, an antigen locally present in an organ, etc. In some instances, immunosuppression may be performed globally e.g., by using an antigen present globally to activate a chimeric polypeptide of the instant disclosure. In some instances, a subject in need of immunosuppression according to the herein described method may be a subject with an autoimmune disease.

As will be readily understood, the methods of treating described herein may, in some instances, be combined with one or more conventional treatments. For example, in the case of oncology, the methods described herein may, in some instances, be combined with a conventional cancer therapy including but not limited to e.g., conventional chemotherapy, conventional radiation therapy, conventional immunotherapy, surgery, etc. In some instances, the methods described herein may be used before or after a conventional therapy. For example, the methods described herein may be used as an adjuvant therapy, e.g., after a subject has seen improvement from a conventional therapy, or may be used when a subject has not responded to a conventional therapy. In some instances, the methods described herein may be used prior to an additional therapy, e.g., to prepare a subject for an additional therapy, e.g., a conventional therapy as described herein.

Conventional cancer therapies also include targeted therapies for cancer including but not limited to e.g., Ado-trastuzumab emtansine (Kadcyla) targeting HER2 (ERBB2/neu) (approved for use in Breast cancer); Afatinib (Gilotrif) targeting EGFR (HER1/ERBB1), HER2 (ERBB2/neu) (approved for use in Non-small cell lung cancer); Aldesleukin (Proleukin) targeting (approved for use in Renal cell carcinoma, Melanoma); Alectinib (Alecensa) targeting ALK (approved for use in Non-small cell lung cancer); Alemtuzumab (Campath) targeting CD52 (approved for use in B-cell chronic lymphocytic leukemia); Atezolizumab (Tecentriq) targeting PD-L1 (approved for use in Urothelial carcinoma, Non-small cell lung cancer); Avelumab (Bavencio) targeting PD-L1 (approved for use in Merkel cell carcinoma); Axitinib (Inlyta) targeting KIT, PDGFRβ, VEGFR1/2/3 (approved for use in Renal cell carcinoma); Belimumab (Benlysta) targeting BAFF (approved for use in Lupus erythematosus); Belinostat (Beleodaq) targeting HDAC (approved for use in Peripheral T-cell lymphoma); Bevacizumab (Avastin) targeting VEGF ligand (approved for use in Cervical cancer, Colorectal cancer, Fallopian tube cancer, Glioblastoma, Non-small cell lung cancer, Ovarian cancer, Peritoneal cancer, Renal cell carcinoma); Blinatumomab (Blincyto) targeting CD19/CD3 (approved for use in Acute lymphoblastic leukemia (precursor B-cell)); Bortezomib (Velcade) targeting Proteasome (approved for use in Multiple myeloma, Mantle cell lymphoma); Bosutinib (Bosulif) targeting ABL (approved for use in Chronic myelogenous leukemia); Brentuximab vedotin (Adcetris) targeting CD30 (approved for use in Hodgkin lymphoma, Anaplastic large cell lymphoma); Brigatinib (Alunbrig) targeting ALK (approved for use in Non-small cell lung cancer (ALK+)); Cabozantinib (Cabometyx, Cometriq) targeting FLT3, KIT, MET, RET, VEGFR2 (approved for use in Medullary thyroid cancer, Renal cell carcinoma); Carfilzomib (Kyprolis) targeting Proteasome (approved for use in Multiple myeloma); Ceritinib (Zykadia) targeting ALK (approved for use in Non-small cell lung cancer); Cetuximab (Erbitux) targeting EGFR (HER1/ERBB1) (approved for use in Colorectal cancer, Squamous cell cancer of the head and neck); Cobimetinib (Cotellic) targeting MEK (approved for use in Melanoma); Crizotinib (Xalkori) targeting ALK, MET, ROS1 (approved for use in Non-small cell lung cancer); Dabrafenib (Tafinlar) targeting BRAF (approved for use in Melanoma, Non-small cell lung cancer); Daratumumab (Darzalex) targeting CD38 (approved for use in Multiple myeloma); Dasatinib (Sprycel) targeting ABL (approved for use in Chronic myelogenous leukemia, Acute lymphoblastic leukemia); Denosumab (Xgeva) targeting RANKL (approved for use in Giant cell tumor of the bone); Dinutuximab (Unituxin) targeting B4GALNT1 (GD2) (approved for use in Pediatric neuroblastoma); Durvalumab (Imfinzi) targeting PD-L1 (approved for use in Urothelial carcinoma); Elotuzumab (Empliciti) targeting SLAMF7 (CS1/CD319/CRACC) (approved for use in Multiple myeloma); Enasidenib (Idhifa) targeting IDH2 (approved for use in Acute myeloid leukemia); Erlotinib (Tarceva) targeting EGFR (HER1/ERBB1) (approved for use in Non-small cell lung cancer, Pancreatic cancer); Everolimus (Afinitor) targeting mTOR (approved for use in Pancreatic, gastrointestinal, or lung origin neuroendocrine tumor, Renal cell carcinoma, Nonresectable subependymal giant cell astrocytoma, Breast cancer); Gefitinib (Iressa) targeting EGFR (HER1/ERBB1) (approved for use in Non-small cell lung cancer); Ibritumomab tiuxetan (Zevalin) targeting CD20 (approved for use in Non-Hodgkin's lymphoma); Ibrutinib (Imbruvica) targeting BTK (approved for use in Mantle cell lymphoma, Chronic lymphocytic leukemia, Waldenstrom's macroglobulinemia); Idelalisib (Zydelig) targeting PI3K8 (approved for use in Chronic lymphocytic leukemia, Follicular B-cell non-Hodgkin lymphoma, Small lymphocytic lymphoma); Imatinib (Gleevec) targeting KIT, PDGFR, ABL (approved for use in GI stromal tumor (KIT+), Dermatofibrosarcoma protuberans, Multiple hematologic malignancies); Ipilimumab (Yervoy) targeting CTLA-4 (approved for use in Melanoma); Ixazomib (Ninlaro) targeting Proteasome (approved for use in Multiple Myeloma); Lapatinib (Tykerb) targeting HER2 (ERBB2/neu), EGFR (HER1/ERBB1) (approved for use in Breast cancer (HER2+)); Lenvatinib (Lenvima) targeting VEGFR2 (approved for use in Renal cell carcinoma, Thyroid cancer); Midostaurin (Rydapt) targeting FLT3 (approved for use in acute myeloid leukemia (FLT3+)); Necitumumab (Portrazza) targeting EGFR (HER1/ERBB1) (approved for use in Squamous non-small cell lung cancer); Neratinib (Nerlynx) targeting HER2 (ERBB2/neu) (approved for use in Breast cancer); Nilotinib (Tasigna) targeting ABL (approved for use in Chronic myelogenous leukemia); Niraparib (Zejula) targeting PARP (approved for use in Ovarian cancer, Fallopian tube cancer, Peritoneal cancer); Nivolumab (Opdivo) targeting PD-1 (approved for use in Colorectal cancer, Head and neck squamous cell carcinoma, Hodgkin lymphoma, Melanoma, Non-small cell lung cancer, Renal cell carcinoma, Urothelial carcinoma); Obinutuzumab (Gazyva) targeting CD20 (approved for use in Chronic lymphocytic leukemia, Follicular lymphoma); Ofatumumab (Arzerra, HuMax-CD20) targeting CD20 (approved for use in Chronic lymphocytic leukemia); Olaparib (Lynparza) targeting PARP (approved for use in Ovarian cancer); Olaratumab (Lartruvo) targeting PDGFRα (approved for use in Soft tissue sarcoma); Osimertinib (Tagrisso) targeting EGFR (approved for use in Non-small cell lung cancer); Palbociclib (Ibrance) targeting CDK4, CDK6 (approved for use in Breast cancer); Panitumumab (Vectibix) targeting EGFR (HER1/ERBB1) (approved for use in Colorectal cancer); Panobinostat (Farydak) targeting HDAC (approved for use in Multiple myeloma); Pazopanib (Votrient) targeting VEGFR, PDGFR, KIT (approved for use in Renal cell carcinoma); Pembrolizumab (Keytruda) targeting PD-1 (approved for use in Classical Hodgkin lymphoma, Melanoma, Non-small cell lung cancer (PD-L1+), Head and neck squamous cell carcinoma, Solid tumors (MSI-H)); Pertuzumab (Perjeta) targeting HER2 (ERBB2/neu) (approved for use in Breast cancer (HER2+)); Ponatinib (Iclusig) targeting ABL, FGFR1-3, FLT3, VEGFR2 (approved for use in Chronic myelogenous leukemia, Acute lymphoblastic leukemia); Ramucirumab (Cyramza) targeting VEGFR2 (approved for use in Colorectal cancer, Gastric cancer or Gastroesophageal junction (GEJ) adenocarcinoma, Non-small cell lung cancer); Regorafenib (Stivarga) targeting KIT, PDGFRβ, RAF, RET, VEGFR1/2/3 (approved for use in Colorectal cancer, Gastrointestinal stromal tumors, Hepatocellular carcinoma); Ribociclib (Kisqali) targeting CDK4, CDK6 (approved for use in Breast cancer (HR+, HER2−)); Rituximab (Rituxan, Mabthera) targeting CD20 (approved for use in Non-Hodgkin's lymphoma, Chronic lymphocytic leukemia, Rheumatoid arthritis, Granulomatosis with polyangiitis); Rituximab/hyaluronidase human (Rituxan Hycela) targeting CD20 (approved for use in Chronic lymphocytic leukemia, Diffuse large B-cell lymphoma, Follicular lymphoma); Romidepsin (Istodax) targeting HDAC (approved for use in Cutaneous T-cell lymphoma, Peripheral T-cell lymphoma); Rucaparib (Rubraca) targeting PARP (approved for use in Ovarian cancer); Ruxolitinib (Jakafi) targeting JAK1/2 (approved for use in Myelofibrosis); Siltuximab (Sylvant) targeting IL-6 (approved for use in Multicentric Castleman's disease); Sipuleucel-T (Provenge) targeting (approved for use in Prostate cancer); Sonidegib (Odomzo) targeting Smoothened (approved for use in Basal cell carcinoma); Sorafenib (Nexavar) targeting VEGFR, PDGFR, KIT, RAF (approved for use in Hepatocellular carcinoma, Renal cell carcinoma, Thyroid carcinoma); Temsirolimus (Torisel) targeting mTOR (approved for use in Renal cell carcinoma); Tositumomab (Bexxar) targeting CD20 (approved for use in Non-Hodgkin's lymphoma); Trametinib (Mekinist) targeting MEK (approved for use in Melanoma, Non-small cell lung cancer); Trastuzumab (Herceptin) targeting HER2 (ERBB2/neu) (approved for use in Breast cancer (HER2+), Gastric cancer (HER2+)); Vandetanib (Caprelsa) targeting EGFR (HER1/ERBB1), RET, VEGFR2 (approved for use in Medullary thyroid cancer); Vemurafenib (Zelboraf) targeting BRAF (approved for use in Melanoma); Venetoclax (Venclexta) targeting BCL2 (approved for use in Chronic lymphocytic leukemia); Vismodegib (Erivedge) targeting PTCH, Smoothened (approved for use in Basal cell carcinoma); Vorinostat (Zolinza) targeting HDAC (approved for use in Cutaneous T-cell lymphoma); Ziv-aflibercept (Zaltrap) targeting PIGF, VEGFA/B (approved for use in Colorectal cancer); and the like.

In some instances, the methods of the instant disclosure may be used without any additional conventional therapy including e.g., where the method described herein is the sole method used to treat the subject. For example, in the case of oncology, the methods described herein may, in some instances, be the sole method used to treat the subject for a cancer.

### Chimeric Polypeptides

The present disclosure provides proteolytically cleavable chimeric polypeptides. The chimeric polypeptides of the instant disclosure may generally include: a) an extracellular domain comprising a specific binding member; b) a proteolytically cleavable Notch receptor polypeptide comprising one or more proteolytic cleavage sites; and c) an intracellular domain. Binding of the specific binding member by its binding partner generally induces cleavage of the proteolytically cleavable Notch receptor at the one or more proteolytic cleavage sites, thereby releasing the intracellular domain. Release of the intracellular domain may modulate an activity of a cell or generally trigger the production of a payload that is contained within the cell, expressed on the cell surface or secreted. The chimeric polypeptides of the instant disclosure will generally include at least one sequence that is heterologous to the Notch receptor polypeptide (i.e., is not derived from a Notch receptor), including e.g., where the extracellular domain is heterologous to the Notch receptor polypeptide, where the intracellular domain is heterologous to the Notch receptor polypeptide, where both the extracellular domain and the intracellular domain are heterologous to the Notch receptor polypeptide, etc.

Domains, e.g., the extracellular domain, the Notch receptor polypeptide regulatory domain, the intracellular domain, etc., may be joined directly, i.e., with no intervening amino acid residues or may include a peptide linker that joins two domains. Peptide linkers may be synthetic or naturally derived including e.g., a fragment of a naturally occurring polypeptide.

A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

### Extracellular Domains

Proteolytically cleavable chimeric polypeptides of the instant disclosure will generally include an extracellular domain that includes a specific binding member that specifically binds to a specific binding partner. Binding of the specific binding member to its specific binding partner triggers proteolytic cleavage of the Notch receptor polypeptide, releasing the intracellular domain which modulates an activity of the cell expressing the chimeric polypeptide.

The specific binding member of the extracellular domain generally determines the specificity of the chimeric polypeptide. In some instances, a chimeric polypeptide may be referred according to its specificity as determined based on its specific binding member. For example, a specific binding member having binding partner “X” may be referred to as an X chimeric polypeptide or an anti-X chimeric polypeptide.

Any convenient specific binding pair, i.e., specific binding member and specific binding partner pair, may find use in the chimeric polypeptides of the instant disclosure including but not limited to e.g., antigen-antibody pairs, ligand receptor pairs, scaffold protein pairs, etc. In some instances, the specific binding member may be an antibody and its binding partner may be an antigen to which the antibody specifically binds. In some instances, the specific binding member may be a receptor and its binding partner may be a ligand to which the receptor specifically binds. In some instances, the specific binding member may be a scaffold protein and its binding partner may be a protein to which the scaffold protein specifically binds.

In some cases, the specific binding member of the chimeric polypeptide is an antibody. The antibody can be any antigen-binding antibody-based polypeptide, a wide variety of which are known in the art. In some instances, the specific binding member is or includes a monoclonal antibody, a single chain Fv (scFv), a Fab, etc. Other antibody based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

Where the specific binding member of a chimeric polypeptide of the present disclosure is an antibody-based binding member, the chimeric polypeptide can be activated in the presence of a binding partner to the antibody-based binding member, including e.g., an antigen specifically bound by the antibody-based binding member. In some instances, antibody-based binding member may be defined, as is commonly done in the relevant art, based on the antigen bound by the antibody-based binding member, including e.g., where the antibody-based binding member is described as an “anti-” antigen antibody, e.g., an anti-CD19 antibody. Accordingly, antibody-based binding members suitable for inclusion in a chimeric polypeptide of the present disclosure can have a variety of antigen-binding specificities.

In some cases, the antigen-binding domain is specific for a cancer antigen, i.e., an antigen expressed by (synthesized by) a neoplasia or cancer cell, i.e., a cancer cell associated antigen or a cancer (or tumor) specific antigen.

A cancer cell associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

A cancer cell specific antigen can be an antigen specific for cancer and/or a particular type of cancer or cancer cell including e.g., a breast cancer cell, a B cell lymphoma, a pancreatic cancer, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer (or tumor) specific antigen is generally not expressed by non-cancerous cells (or non-tumor cells). In some instances, a cancer (or tumor) specific antigen may be minimally expressed by one or more non-cancerous cell types (or non-tumor cell types). By “minimally expressed” is meant that the level of expression, in terms of either the per-cell expression level or the number of cells expressing, minimally, insignificantly or undetectably results in binding of the specific binding member to non-cancerous cells expressing the antigen.

In some instances, a specific binding member of a chimeric polypeptide may specifically bind a target comprising a fragment of a protein (e.g., a peptide) in conjunction with a major histocompatibility complex (MHC) molecule. As MHC molecules present peptide fragments of both intracellularly expressed and extracellularly expressed proteins, specific binding members directed to MHC-peptide complexes allows for the targeting of intracellular antigens as well as extracellularly expressed antigens.

Intracellularly expressed target proteins (e.g., cytoplasmically expressed (i.e., cytoplasmic proteins), nuclearly expressed (i.e., nuclear proteins), etc.) may be referred to as intracellular antigens (e.g., cytoplasmic antigens, nuclear antigens, etc.). Accordingly, specific binding members of the subject disclosure may be specific for intracellular antigen fragments complexed with MHC, e.g., a peptide-MHC complex, also, in some instances, described as a human leukocyte antigen (HLA)-peptide complex.

All endogenous cellular proteins (host or pathogen) are processed into short peptides for display at the cell surface in association with HLA molecules. Peptide-HLA class I complexes displayed on the cell surface play an important role in the T-cell mediated immune response. The approximately 9-residue long peptides originate from proteins that are digested by the proteasome inside the cell. Depending on whether the T-cell receptor recognizes a peptide as self or non-self, an immune response may be initiated. Peptide-HLA complexes displayed specifically on the surface of cancer cells provide an excellent opportunity to develop targeted cancer therapeutics, including engineered T-cells or “TCR-like” antibodies. The advent of various technologies, including e.g., MHC based tetramer technology, have advanced the ability to develop TCR-like anti-HLA/peptide specific antibodies.

In some instances, the binding partner of a specific binding member of the subject chimeric polypeptides may include peptide-MHC or HLA/peptide complexes. In some instances, the specific binding member of the subject chimeric polypeptides is specific for a MHC class I MHC-peptide complex including e.g., a HLA-A/peptide complex, a HLA-B/peptide complex or a HLA-C/peptide complex. In some instances, the specific binding member of the subject chimeric polypeptides is specific for a MHC class II MHC-peptide complex including e.g., a HLA-DPA1/peptide complex, a HLA-DPB 1/peptide complex, a HLA-DQA1/peptide complex, a HLA-DQB1/peptide complex, a HLA-DRA/peptide complex or a HLA-DRB1/peptide complex. In some instances, the specific binding member of the subject chimeric polypeptides is specific for a MHC class III MHC-peptide complex.

Peptide-MHC Binding partners will generally include a target protein fragment peptide presented in the context of MHC. Such peptides vary in size depending on numerous factors including e.g., the class of MHC molecule to which they are bound. For example, class I MHC associated peptides are generally 9 aa in length but may vary in size including less than about 9 aa or more than about 9 aa including but not limited to e.g., 8 aa or 10 aa. Whereas, class II MHC associated peptides may also vary in size from about 13 aa to about 25 aa, including but not limited to e.g., 13 aa, 14 aa, 15 aa, 16 aa, 17 aa, 18 aa, 19 aa, 20 aa, 21 aa, 22 aa, 23 aa, 24 aa or 25 aa.

Exemplary protein targets to which a specific binding member targeting a peptide-MHC complex may be directed as well as exemplary peptides in the context of MHC for each protein target are provided in Table 1 below.

TABLE 1
anti-peptide-MHC targets

| Target | Exemplary Peptides | HLA | References |
|---|---|---|---|
| WT1 | RMFPNAPYL (SEQ ID NO: 18) | HLA-A2 | Leukemia. (2015) 29(11): 2238-47 |
| KRAS and | KLVVVGAGGV (SEQ ID NO: 19); | HLA-A2; | Proc Natl Acad Sci USA. (2015) 112(32) |
| KRAS mutants | KLVVVGAVGV (SEQ ID NO: 20); | HLA-A3 | |
| e.g., G12V & | KLVVVGACGV (SEQ ID NO: 21); | | |
| G12C) | KLVVVGADGV (SEQ ID NO: 22); | | |
| | VVGAVGVGK (SEQ ID NO: 23); | | |
| | VVGACGVGK (SEQ ID NO: 24); | | |
| | VVGAGGVGK (SEQ ID NO: 25) | | |
| EGFP and EGFP | KITDFGLAK (SEQ ID NO: 26); | HLA-A3 | Proc Natl Acad Sci USA. (2015) 112(32) |
| mutants (e.g., | KITDFGRAK (SEQ ID NO: 27); | | |
| L858R) | | | |
| PR1/Proteinase 3 | VLQELNVTV (SEQ ID NO: 28) | HLA-A2 | Cytotherapy. (2016) 18(8): 985-94 |
| MAGE-A1 | EADPTGHSY (SEQ ID NO: 29) | HLA-A1 | Blood. (2011) 117(16): 4262-4272 |
| MAGE3 | FLWGPRALV (SEQ ID NO: 30) | HLA-A2 | Eur J Immunol (2005) 35: 2864-2875 |
| P53 | LLGRNSFEV (SEQ ID NO: 31); | HLA-A2 | Gene Ther. (2001) 8(21): 1601-8 |
| | STTPPPGTRV (SEQ ID NO: 32) | | PLoS One (2017) 12: 1-16 |
| | RMPEAAPPV (SEQ ID NO: 33) | | |
| | GLAPPQHLIRV (SEQ ID NO: 34) | | |
| MART-1 | ELAGIGILTV (SEQ ID NO: 35) | HLA-A2 | Biomark Med. (2010) 4(4): 496-7 |
| | EAAGIGILTV (SEQ ID NO: 36) | | Eur J Immunol (2007) 37: 2008-2017 |
| gp100 | IMDQVPFSV (SEQ ID NO: 37) | HLA-A2 | Biomark Med. (2010) 4(4): 496-7 |
| | KTWGQYWQV (SEQ ID NO: 38) | | J Immunol (2002) 169: 4399-407 |
| | YLEPGPVTV (SEQ ID NO: 39) | | U.S. Pat. Pub. No. US20030223994 |
| | YLEPGPVTA (SEQ ID NO: 40) | | J Immunol (2003) 171: 2197-2207 |
| | ITDQVPFSV (SEQ ID NO: 41) | | |
| CMV pp65 | NLVPMVATV (SEQ ID NO: 42) | HLA-A2 | Biomark Med. (2010) 4(4): 496-7 |
| HIV Vpr | AIIRILQQL (SEQ ID NO: 43) | HLA-A2 | Biomark Med. (2010) 4(4): 496-7 |
| HA-1H | VLHDDLLEA (SEQ ID NO: 44); | HLA-A2 | Biomark Med. (2010) 4(4): 496-7 |
| | VLRDDLLEA (SEQ ID NO: 45) | | |
| NY-ESO-1 | SLLMWITQV (SEQ ID NO: 46) | HLA-A2 | Gene Ther. (2014) 21(6): 575-84 |
| EBNA3C | LLDFVRFMGV (SEQ ID NO: 47) | HLA-A2 | Proc Natl Acad Sci USA. (2009) |
| | | | 106(14): 5784-8 |
| AFP | FMNKFIYEI (SEQ ID NO: 48) | HLA-A2 | Cancer Gene Ther. (2012) 19(2): 84-100 |
| Her2 | KIFGSLAFL (SEQ ID NO: 49) | HLA-A2 | Clin Cancer Res. (2016) pii: clincanres |
| | | | 1203.2016 |
| hCG-beta | GVLPALPQV (SEQ ID NO: 50) | HLA-A2 | J Natl Cancer Inst. (2013) 105(3): 202-18 |
| | TMTRVLQGV (SEQ ID NO: 51) | | Vaccine (2008) 26: 3092-3102 |
| HBV Env183-91 | FLLTRILTI (SEQ ID NO: 52) | HLA-A2 | J Immunol. (2006) 177(6): 4187-95 |
| hTERT | ILAKFLHWL (SEQ ID NO: 53) | HLA-A2 | Cancer Res (2002) 62: 3184-3194 |
| | RLVDDFLLV (SEQ ID NO: 54) | | |
| MUC1 | LLLTVLTVV (SEQ ID NO: 55) | HLA-A2 | Cancer Res (2002) 62: 5835-5844 |
| TARP | FLRNFSLML (SEQ ID NO: 56) | HLA-A2 | Eur J Immunol (2008) 38: 1706-1720 |
| Tyrosinase | YMDGTMSQV (SEQ ID NO: 57) | HLA-A2 | J Immunol (2009) 182: 6328-41 |
| p68 | YLLPAIVHI (SEQ ID NO: 58) | HLA-A2 | Cancer Immunol Immunother (2010) |
| | | | 59: 563-573 |
| MIF | FLSELTQQL (SEQ ID NO: 59) | HLA-A2 | J Immunol (2011) 186: 6607 |
| PRAME | ALYVDSLFFL (SEQ ID NO: 60) | HLA-A2 | J Clin Invest (2017)1-14 |

In some instances, the specific binding member of a proteolytically cleavable chimeric polypeptide of the instant disclosure specifically binds a peptide-MHC having an intracellular cancer antigen peptide of Table 1. In some instances, the specific binding member of a proteolytically cleavable chimeric polypeptide of the instant disclosure specifically binds a WT1 peptide-MHC. In some instances, the specific binding member of a proteolytically cleavable chimeric polypeptide of the instant disclosure specifically binds a NY-ESO-1 peptide-MHC.

In some instances, the specific binding member of a proteolytically cleavable chimeric polypeptide of the instant disclosure is an antibody (e.g., a scFv) that specifically binds a peptide-MHC having an intracellular cancer antigen peptide of Table 1. In some instances, the specific binding member of a proteolytically cleavable chimeric polypeptide of the instant disclosure is an antibody (e.g., a scFv) that specifically binds a WT1 peptide-MHC.

In some instances, an antibody that specifically binds to a WT1 peptide-MHC includes one or more or all of the following heavy-chain (VH) CDR sequences: SYAMS (SEQ ID NO:61), QIDPWGQETLYADSVKG (SEQ ID NO:62) and LTGRFDY (SEQ ID NO:63).

In some instances, an antibody that specifically binds to a WT1 peptide-MHC includes one or more or all of the following light-chain (VL) CDR sequences: RASQSISSYLN (SEQ ID NO:64), SASQLQS (SEQ ID NO:65) and QQGPGTPNT (SEQ ID NO:66).

In some instances, an antibody that specifically binds to a WT1 peptide-MHC is an scFv having the following amino acid sequence:

(SEQ ID NO: 67)
EVQLLESGGGLVQPGGSLRLSCAASGFTFSSYAMSWVRQAPGKGLEWVSQ
IDPWGQETLYADSVKGRFTISRDNSKNTLYLQMNSLRAEDTAVYYCAKLT
GRFDYWGQGTLVTVSSGGGGSGGGGSGGGGSTDIQMTQSPSSLSASVGDR
VTITCRASQSISSYLNWYQQKPGKAPKLLIYSASQLQSGVPSRFSGSGSG
TDFTLTISSLQPEDFATYYCQQGPGTPNTFGQGTKVEIKRA.

In some instances, the specific binding member of a proteolytically cleavable chimeric polypeptide of the instant disclosure is an antibody (e.g., a scFv) that specifically binds a NY-ESO-1 peptide-MHC.

In some instances, the specific binding member of a proteolytically cleavable chimeric polypeptide of the instant disclosure is an antibody (e.g., a scFv) that specifically binds a KRAS-G12V peptide-MHC (e.g., anti-HLA-A2/KRAS-G12V scFv).

In some instances, an antibody that specifically binds to a KRAS-G12V peptide-MHC is an scFv having the following amino acid sequence:

(SEQ ID NO: 68)
DIQMTQSPSSLSASVGDRVTITCRASQDVNTAVAWYQQKPGKAPKLLIYS
ASFLYSGVPSRFSGSRSGTDFTLTISSLQPEDFATYYCQQYYYYPPTFGQ
GTKVEIKRTGGGSGGGGSGGGASEVQLVESGGGLVQPGGSLRLSCAASGF
NINGSYIHWVRQAPGKGLEWVAYIDPETGYSRYADSVKGRFTISADTSKN
TAYLQMNSLRAEDTAVYYCSRDSASDAMDVWGQGTLVTVSS.

In some instances, the specific binding member of a proteolytically cleavable chimeric polypeptide of the instant disclosure is an antibody (e.g., a scFv) that specifically binds a KRAS-G12V/C peptide-MHC (e.g., anti-HLA-A2/KRAS-G12V/C scFv).

In some instances, an antibody that specifically binds to a KRAS-G12V/C peptide-MHC is an scFv having the following amino acid sequence:

(SEQ ID NO: 69)
DIQMTQSPSSLSASVGDRVTIACRASQDVNTAVAWYQQKPGKAPKLLIYS
ASFLYSGVPSRFSGSRSGTDFTLTISSLQPEDFATYYCQQYYYYPPTFGQ
GTKVEIKRTGGGSGGGGSGGGASEVQLVESGGGLVQPGGSLRLSCAASGF
HINGSYIHWVRQAPGKGLKWVAYIDPETGYSRYADSVKGRFAISADMSKN
TAYLQMNSLRAEDTAVYYCSRDSASDAMDVWGQGTLVTVSS.

In some instances, the specific binding member of a proteolytically cleavable chimeric polypeptide of the instant disclosure is an antibody (e.g., a scFv) that specifically binds an EGFR-L858R peptide-MHC (e.g., anti-HLA-A3/EGFR-L858R scFv).

In some instances, an antibody that specifically binds to an EGFR-L858R peptide-MHC is an scFv having the following amino acid sequence:

(SEQ ID NO: 70)
DIQMTQSPSSLSASVGDRVTITCRASQDVNTAVAWYQQKPGKAPKLLIYS
ASFLYSGVPSRFSGSRSGTDFTLTISSLQPEDFATYYCQQYYSYPPTFGQ
GTKVEIKRTGGGSGGGGSGGGASEVQLVESGGGLVQPGGSLRLSCAASGF
NITSSYIHWVRQAPGKGLEWVAYISPEDGYARHADSVKGRFTISADTSKN
TAYLQMNSLRAEDTAVYYCSRDDTYYYSAMDVWGQGTLVTVSS.

In some instances, the specific binding member (e.g., an antibody, scFv, etc.) of a proteolytically cleavable chimeric polypeptide of the instant disclosure specifically binds a peptide-MHC described in Dhanik et al. BMC Bioinformatics (2016) 17:286, the disclosure of which is incorporated herein by reference in its entirety, including but not limited to e.g., a NLRP4 peptide (e.g., HLSPIDCEV (SEQ ID NO: 71))-MHC complex, a UMODL1 peptide (e.g., LTSMWSPAV (SEQ ID NO:72))-MHC complex, a NLRP4 peptide (e.g., HLDHPHPAV (SEQ ID NO:73))-MHC complex, a MAGEC2 peptide (e.g., SLSVMSSNV (SEQ ID NO:74))-MHC complex, a NLRP4 peptide (e.g., MMAWSDNKI (SEQ ID NO: 75))-MHC complex, a COX7B2 peptide (e.g., TQIGIEWNL (SEQ ID NO:76))-MHC complex, a NLRP4 peptide (e.g., CLFEMQDPA (SEQ ID NO:77))-MHC complex, a UMODL1 peptide (e.g., YLSHPSCNV (SEQ ID NO:78))-MHC complex, a COX7B2 peptide (e.g., GIEWNLSPV (SEQ ID NO: 79))-MHC complex, a MAGEA11 peptide (e.g., GLGCSPASI (SEQ ID NO:80))-MHC complex, a RPE65 peptide (e.g., RQAFEFPQI (SEQ ID NO:81))-MHC complex, a NLRP4 peptide (e.g., GMWTDTFEF (SEQ ID NO:82))-MHC complex, a TRIM51 peptide (e.g., YLNWQDTAV (SEQ ID NO: 83))-MHC complex, a MAGEA11 peptide (e.g., VLWGPITQI (SEQ ID NO:84))-MHC complex, a NLRP4 peptide (e.g., TLDHTGVVV (SEQ ID NO:85))-MHC complex, a RPE65 peptide (e.g., TMGVWLHIA (SEQ ID NO:86))-MHC complex, a MAGEC2 peptide (e.g., KVWVQGHYL (SEQ ID NO: 87))-MHC complex, a UMODL1 peptide (e.g., KINCNNFRL (SEQ ID NO:88))-MHC complex, etc.

Any suitable anti-peptide-MHC specific binding member (e.g., anti-peptide-MHC antibody) may find use as the specific binding member of an extracellular domain of a subject chimeric polypeptide as described herein, including but not limited to e.g., those suitable anti-peptide-MHC binding members and antibodies, as well as antibodies to those suitable epitopes, described in e.g.: U.S. Pat. Nos. 6,042,831; 6,252,052; 6,291,430; 6,602,510; 7,157,091; 7,622,569; 7,632,923; 7,638,124; 7,718,777; 8,119,139; 8,647,629; 8,815,528; 8,961,985; 9,023,348; 9,040,669; 9,074,000; 9,095,533; 9,334,317; U.S. Patent Application Pub. Nos: 20070092530; 20090042285; 20090226474; 20090304679; 20100062001; 20100111957; 20100158927; 20100158931; 20110020357; 20110033473; 20110293623; 20110318369; 20120141517; 20120294874; 20130101594; 20140024809; 20140065708; 20140271644; 20140294841; 20140296492; 20140363440; 20150125477; 20150125478; 20150259436; 20150320848; 20150322154; 20150368298; 20160017031; 20160168200; PCT Pub. Nos: WO2002014870; WO2003068201; WO2005120166; WO2007030451; WO2007143104; WO2008120202; WO2008120203; WO2009108372; WO2009125394; WO2009125395; WO2009138236; WO2010037514; WO2010106431; WO2011062560; WO2012007950; WO2012007951; WO2012017003; WO2012109659; WO2012135854; WO2014011489; WO2014143835; WO2015018805; WO2015063302; WO2015070061; WO2015070078; WO2015090229; WO2015130766; WO2015142675; WO2015169945; WO2015193359; WO2015199617; WO2016102272; and the like, the disclosures of which are incorporated herein by reference in their entirety.

In some instances, an anti-peptide-MHC specific binding member (e.g., an anti-peptide-MHC antibody) that may find use as the specific binding member of an extracellular domain of a subject chimeric polypeptide as described herein, may include but is not limited to e.g., those suitable anti-peptide-MHC binding members and antibodies, as well as antibodies to those suitable epitopes, described in e.g.: T Cells Expressing Cars Directed Against HLA-0201 eRmf WT-1 Peptide Complex Can Effectively Eradicate WT1+ A0201+ Tumor Cells in-Vitro. (2014) Biol Blood Marrow Transplant; A novel TCR-like CAR with specificity for PR1/HLA-A2 effectively targets myeloid leukemia in vitro when expressed in human adult peripheral blood and cord blood T cells. (2016) Cytotherapy; Functional Comparison of Engineered T Cells Carrying a Native TCR versus TCR-like Antibody-Based Chimeric Antigen Receptors Indicates Affinity/Avidity Thresholds. (2014) Journal of Immunology; Affinity maturation of T-cell receptor-like antibodies for Wilms tumor 1 peptide greatly enhances therapeutic potential. (2015) Leukemia; Construction and molecular characterization of a T-cell receptor-like antibody and CAR-T cells specific for minor histocompatibility antigen HA-1H. (2014) Gene Therapy; Targeting the Intracellular WT1 Oncogene Product with a Therapeutic Human Antibody. (2013) Science Translational Medicine; A phage display selected Fab fragment with MHC class I-restricted specificity for MAGE-A1 allows for retargeting of primary human T lymphocytes. (2001) Gene Therapy; Therapeutic bispecific T-cell engager antibody targeting the intracellular oncoprotein WT1. (2015) Nature Biotechnology; Generation of MANAbodies specific to HLA-restricted epitopes encoded by somatically mutated genes. (2015) PNAS; Antitumor Activity of a Monoclonal Antibody targeting Major Histocompatibility complex class i-Her2 Peptide complexes. (2013) Journal of the National Cancer Institute; Anti-melanoma activity of T cells redirected with a TCR-like chimeric antigen receptor. (2014) Scientific Reports; and the like.

In some instances, a specific binding member of a chimeric polypeptide of the instant disclosure may include a T-cell receptor-like anti-peptide-MHC antibody including but not limited to e.g., those described in e.g., Dahan & Reiter. Expert Rev Mol Med. (2012) 14:e6 and/or Cohen & Reiter. Antibodies (2013), 2:517-534, the disclosures of which are incorporated herein by reference in their entirety, including but not limited to e.g.: clone G2D12 targeting HLA-A2/gp100-154; clone 1A7 targeting HLA-A2/gp100-209; clone G1 targeting HLA-A2/gp100-209; clone 2F1 targeting HLA-A2/gp100-280; clone TA2 targeting HLA-A2/Tyrosinase-369; clone CAG10, CLA12 targeting HLA-A2/MART-1-26; clone Fab-G8 targeting HLA-A1/MAGE-A1; clone Fab-Hyb3 targeting HLA-A1/MAGE-A1; clone 7D4 targeting HLA-A2/MAGE3-271; clone RL6A targeting HLA-A2/p68 RNA helicase-128; clone 3M4E5, 3M4F4 targeting HLA-A2/NYESO-1-157; clone T1 targeting HLA-A2/NYESO-1-157; clone D2 targeting HLA-A2/TARP-29; clone RL4B, 1B10 targeting HLA-A2/hCGB-47; clone 3F9 targeting HLA-A2/hCGB-40; clone 1B8 targeting HLA-A2/Her2-369; clone 8F4 targeting HLA-A2/PR1; clone F2 targeting HLA-A2/WT1-db126; clone M3A1, M3B8 targeting HLA-A2/MUC-1-D6-13; clone 4A9, 4G9 targeting HLA-A2/telomerase-540; clone 3G3, 3H2 targeting HLA-A2/telomerase-865; clone T3A4, T3D4, T3E3, T3F2, T3D3, T2H9 targeting HLA-A2/TAX-11; clone M1-D1, M1-G8, M1-D12, M1-A2 targeting HLA-A2/M1-58; the clone targeting HLA-A2/ENV-183; clone C3 targeting HLA-C7/Nef-105; clone 4F7 targeting HLA-A2/eIF4G-720; clone scFv #3, scFv #27 targeting HLA-A24/Nef-138; clone H9 targeting HLA-A2/pp65-495; and the like.

In addition to or in exchange of a peptide-MHC binding partner described herein, chimeric polypeptides of the instant disclosure may, in some cases, target a surface expressed antigen. As used herein the term “surface expressed antigen” generally refers to antigenic proteins that are expressed at least partially extracellularly such that at least a portion of the protein is exposed outside the cells and available for binding with a binding partner. Essentially any surface expressed protein may find use as a target of a chimeric polypeptide of the instant disclosure. Non-limiting examples of surface expressed antigens include but are not limited to e.g., CD19, CD20, CD30, CD38, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), IL-13R-a2, GD2, and the like. Surface expressed antigens that may be targeted also include but are not limited to e.g., those specifically targeted in conventional cancer therapies, including e.g., those targets of the targeted cancer therapeutics described herein.

In some instances, the specific binding member of a chimeric polypeptide of the instant disclosure may target a cancer-associated antigen. In some instances, a specific binding member of the instant disclosure may include an antibody specific for a cancer associated antigen. Non-limiting examples of cancer associated antigens include but are not limited to e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

In some instances, the specific binding member of a chimeric polypeptide of the instant disclosure may target or may include all or a portion of an antibody targeting phosphatase of regenerating liver 3 (PRL-3, also known as PTP4A3), such as e.g., PRL3-zumab as described in Thura et al. (JCI Insight. 2016; 1(9):e87607); the disclosure of which is incorporated herein by reference in its entirety.

In some instances, the extracellular domain of a chimeric polypeptide may include only one specific binding member. In some instances, the extracellular domain of a chimeric polypeptide may by mono-specific.

In some instances, the extracellular domain of a chimeric polypeptide may by multi-specific, including e.g., bispecific. In some instances, a bispecific extracellular domain of a chimeric polypeptide may include a bispecific chimeric binding member, or portion thereof, including e.g., those described herein, including but not limited to e.g., a bispecific antibody. In some instances, a bispecific extracellular domain may include two specific binding domains that are linked, including e.g., directly linked to each other or linked via a linker.

In some instances, the extracellular domain of a chimeric polypeptide may include more than one specific binding member, including two or more specific binding members where the two or more specific binding members may be linked (either directly or indirectly, e.g., through the use of a linker) to each other or they may each be linked (either directly or indirectly, e.g., through the use of a linker) to another component of the chimeric polypeptide.

Multi-specific extracellular domains may recognize or bind to any combination of binding partners and thus may target any combination of targets, including but not limited to e.g., those binding partners and targets described herein. Accordingly, e.g., a bispecific extracellular domain may target two different antigens including but not limited to e.g., two different intracellular antigens, two different extracellular (e.g., surface expressed) antigens or an intracellular antigen and an extracellular (e.g., surface expressed) antigen. In some instances, a bispecific extracellular domain may include two specific binding members, including e.g., two specific binding members described herein, that each bind an antigen, including e.g., an antigen described herein.

The specific binding domains of a multi-specific extracellular domain may each activate the chimeric polypeptide of which they are a part. The specific binding domains of a bispecific extracellular domain may each activate the chimeric polypeptide of which they are a part. In some instances, multi-specific or bispecific binding domains may find use as part of a molecular circuit as described herein including e.g., as an OR-gate of a circuit described herein.

In some instances, the binding partner bound by a specific binding domain may be mutated as compared to the wild-type binding partner. In some instances, a specific binding domain that recognizes a mutated binding partner may not specifically bind the wild-type binding partner. In some instances, a specific binding domain that recognizes a mutated binding partner may bind the wild-type binding partner with lower affinity as compared to its binding affinity with the mutated binding partner.

Any binding partner, including e.g., those described herein, may be mutated or may be a mutated binding partner. Accordingly, a chimeric polypeptide of the instant disclosure may include a specific binding member that specifically binds a mutated (i.e., non-wild-type) binding partner. Non-limiting examples of mutated binding partners include but are not limited to e.g., mutated antigens, mutated cancer antigens, mutated auto-antigens, mutated extracellular antigens, mutated extracellular cancer antigens, mutated extracellular auto-antigens, mutated surface antigens, mutated surface cancer antigens, mutated surface auto-antigens, peptide-MHC complexes presenting a mutated antigen peptide, peptide-MHC complexes presenting a mutated cancer antigen peptide, peptide-MHC complexes presenting a mutated auto-antigen peptide, and the like.

Cancers commonly involve mutated proteins that are associated with the disease. Genes commonly mutated in cancers include e.g., ABI1, ABL1, ABL2, ACKR3, ACSL3, ACSL6, AFF1, AFF3, AFF4, AKAP9, AKT1, AKT2, ALDH2, ALK, AMER1, APC, ARHGAP26, ARHGEF12, ARIDIA, ARID2, ARNT, ASPSCR1, ASXL1, ATF1, ATIC, ATM, ATP1A1, ATP2B3, ATRX, AXIN1, BAP1, BCL10, BCL11A, BCL11B, BCL2, BCL3, BCL6, BCL7A, BCL9, BCOR, BCR, BIRC3, BLM, BMPRIA, BRAF, BRCA1, BRCA2, BRD3, BRD4, BRIP1, BTG1, BUB1B, C15orf65, C2orf44, CACNAID, CALR, CAMTA1, CANT1, CARD11, CARS, CASC5, CASP8, CBFA2T3, CBFB, CBL, CBLB, CBLC, CCDC6, CCNB1IP1, CCND1, CCND2, CCND3, CCNE1, CD274, CD74, CD79A, CD79B, CDC73, CDH1, CDH11, CDK12, CDK4, CDK6, CDKN2A, CDKN2C, CDX2, CEBPA, CEP89, CHCHD7, CHEK2, CHIC2, CHN1, CIC, CIITA, CLIP1, CLP1, CLTC, CLTCL1, CNBP, CNOT3, CNTRL, COL1A1, COL2A1, COX6C, CREB1, CREB3L1, CREB3L2, CREBBP, CRLF2, CRTC1, CRTC3, CSF3R, CTNNB1, CUX1, CYLD, DAXX, DCTN1, DDB2, DDIT3, DDX10, DDX5, DDX6, DEK, DICER1, DNM2, DNMT3A, EBF1, ECT2L, EGFR, EIF3E, EIF4A2, ELF4, ELK4, ELL, ELN, EML4, EP300, EPS15, ERBB2, ERC1, ERCC2, ERCC3, ERCC4, ERCC5, ERG, ETV1, ETV4, ETV5, ETV6, EWSR1, EXT1, EXT2, EZH2, EZR, FAM46C, FANCA, FANCC, FANCD2, FANCE, FANCF, FANCG, FAS, FBXO11, FBXW7, FCGR2B, FCRL4, FEV, FGFR1, FGFR1OP, FGFR2, FGFR3, FH, FHIT, FIP1L1, FLCN, FLI1, FLT3, FNBP1, FOXA1, FOXL2, FOXO1, FOXO3, FOXO4, FOXP1, FSTL3, FUBP1, FUS, GAS7, GATA1, GATA2, GATA3, GMPS, GNA11, GNAQ, GNAS, GOLGA5, GOPC, GPC3, GPHN, H3F3A, H3F3B, HERPUD1, HEY1, HIP1, HIST1H4I, HLA-A, HLF, HMGA1, HMGA2, HNFIA, HNRNPA2B1, HOOK3, HOXA11, HOXA13, HOXA9, HOXC11, HOXC13, HOXD11, HOXD13, HRAS, HSP90AA1, HSP90AB1, IDH1, IDH2, IKZF1, IL2, IL21R, IL6ST, IL7R, IRF4, ITK, JAK1, JAK2, JAK3, JAZF1, JUN, KAT6A, KAT6B, KCNJ5, KDM5A, KDM5C, KDM6A, KDR, KDSR, KIAA1549, KIAA1598, KIF5B, KIT, KLF4, KLF6, KLK2, KMT2A, KMT2C, KMT2D, KRAS, KTN1, LASP1, LCK, LCP1, LHFP, LIFR, LMNA, LMO1, LMO2, LPP, LRIG3, LSM14A, LYL1, MAF, MAFB, MALT1, MAML2, MAP2K1, MAP2K2, MAP2K4, MAX, MDM2, MDM4, MECOM, MED12, MEN1, MET, MITF, MKL1, MLF1, MLH1, MLLT1, MLLT10, MLLT11, MLLT3, MLLT4, MLLT6, MN1, MNX1, MPL, MSH2, MSH6, MSI2, MSN, MTCP1, MUC1, MUTYH, MYB, MYC, MYCL, MYCN, MYD88, MYH11, MYH9, MYO5A, NAB2, NACA, NBN, NCKIPSD, NCOA1, NCOA2, NCOA4, NDRG1, NF1, NF2, NFATC2, NFE2L2, NFIB, NFKB2, NIN, NKX2-1, NONO, NOTCH1, NOTCH2, NPM1, NR4A3, NRAS, NRG1, NSD1, NT5C2, NTRK1, NTRK3, NUMA1, NUP214, NUP98, NUTM1, NUTM2A, NUTM2B, OLIG2, OMD, P2RY8, PAFAH1B2, PALB2, PATZ1, PAX3, PAX5, PAX7, PAX8, PBRM1, PBX1, PCM1, PCSK7, PDCD1LG2, PDE4DIP, PDGFB, PDGFRA, PDGFRB, PER1, PHF6, PHOX2B, PICALM, PIK3CA, PIK3R1, PIM1, PLAG1, PLCG1, PML, PMS1, PMS2, POT1, POU2AF1, POU5F1, PPARG, PPFIBP1, PPP2R1A, PRCC, PRDM1, PRDM16, PRF1, PRKAR1A, PRRX1, PSIP1, PTCH1, PTEN, PTPN11, PTPRB, PTPRC, PTPRK, PWWP2A, RABEP1, RAC1, RAD21, RAD51B, RAF1, RALGDS, RANBP17, RAP1GDS1, RARA, RB1, RBM15, RECQL4, REL, RET, RHOH, RMI2, RNF213, RNF43, ROS1, RPL10, RPL22, RPL5, RPN1, RSPO2, RSPO3, RUNX1, RUNX1T1, SBDS, SDC4, SDHAF2, SDHB, SDHC, SDHD, SEPT5, SEPT6, SEPT9, SET, SETBP1, SETD2, SF3B1, SFPQ, SH2B3, SH3GL1, SLC34A2, SLC45A3, SMAD4, SMARCA4, SMARCB1, SMARCE1, SMO, SOCS1, SOX2, SPECC1, SRGAP3, SRSF2, SRSF3, SS18, SS18L1, SSX1, SSX2, SSX2B, SSX4, SSX4B, STAG2, STAT3, STAT5B, STAT6, STIL, STK11, SUFU, SUZ12, SYK, TAF15, TAL1, TAL2, TBL1XR1, TCEA1, TCF12, TCF3, TCF7L2, TCL1A, TERT, TET1, TET2, TFE3, TFEB, TFG, TFPT, TFRC, THRAP3, TLX1, TLX3, TMPRSS2, TNFAIP3, TNFRSF14, TNFRSF17, TOP1, TP53, TPM3, TPM4, TPR, TRAF7, TRIM24, TRIM27, TRIM33, TRIP11, TRRAP, TSC1, TSC2, TSHR, TTL, U2AF1, UBR5, USP6, VHL, VTI1A, WAS, WHSC1, WHSCIL1, WIFI, WRN, WT1, WWTR1, XPA, XPC, XPO1, YWHAE, ZBTB16, ZCCHC8, ZMYM2, ZNF331, ZNF384, ZNF521 and ZRSR2. In some instances, a specific binding member binds to the mutated version of a gene that is commonly mutated in cancer, including but not limited to e.g., those listed above. In some instances, a specific binding member binds to a peptide-MHC complex presenting a mutated cancer antigen peptide derived from the mutated version of a gene that is commonly mutated in cancer, including but not limited to e.g., those listed above. In some instances, a specific binding member binds to a peptide-MHC complex presenting a mutant KRAS peptide.

In some instances, a binding partner/specific binding member pair may be orthogonalized. As used herein, by “orthogonalized” is meant modified from their original or wild-type form such that the orthogonal pair specifically bind one another but do not specifically or substantially bind the non-modified or wild-type components of the pair. Any binding partner/specific binding pair may be orthogonalized, including but not limited to e.g., those binding partner/specific binding pairs described herein.

Certain extracellular domains and components thereof that may be adapted for use in chimeric polypeptides and the methods and circuits described herein include but are not limited to e.g., those described in PCT Application No. US2016/019188 (Pub. No. WO 2016/138034), the disclosure of which is incorporated herein by reference in its entirety.

### Notch Receptor Polypeptides

As noted above, a chimeric polypeptide of the present disclosure includes a Notch receptor polypeptide. Notch receptor polypeptides of the subject chimeric polypeptides will vary and may range in length from about 50 amino acids or less to about 1000 amino acids or more and will generally include one or more ligand-inducible proteolytic cleavage sites. Notch receptor polypeptides include synthetic receptors containing a Notch regulatory region or a modified form thereof.

In some cases, the Notch receptor polypeptide present in a chimeric polypeptide of the present disclosure has a length of from 50 amino acids (aa) to 1000 aa, e.g., from 50 aa to 75 aa, from 75 aa to 100 aa, from 100 aa to 150 aa, from 150 aa to 200 aa, from 200 aa to 250 aa, from 250 a to 300 aa, from 300 aa to 350 aa, from 350 aa to 400 aa, from 400 aa to 450 aa, from 450 aa to 500 aa, from 500 aa to 550 aa, from 550 aa to 600 aa, from 600 aa to 650 aa, from 650 aa to 700 aa, from 700 aa to 750 aa, from 750 aa to 800 aa, from 800 aa to 850 aa, from 850 aa to 900 aa, from 900 aa to 950 aa, or from 950 aa to 1000 aa. In some cases, the Notch receptor polypeptide present in a chimeric polypeptide of the present disclosure has a length of from 300 aa to 400 aa. In some cases, the Notch receptor polypeptide present in a chimeric polypeptide of the present disclosure has a length of from 300 aa to 350 aa. In some cases, the Notch receptor polypeptide present in a chimeric polypeptide of the present disclosure has a length of from 300 aa to 325 aa. In some cases, the Notch receptor polypeptide present in a chimeric polypeptide of the present disclosure has a length of from 350 aa to 400 aa. In some cases, the Notch receptor polypeptide present in a chimeric polypeptide of the present disclosure has a length of from 750 aa to 850 aa. In some cases, the Notch receptor polypeptide present in a chimeric polypeptide of the present disclosure has a length of from 50 aa to 75 aa. In some cases, the Notch receptor polypeptide present in a chimeric polypeptide of the present disclosure has a length of from 310 aa to 320 aa, e.g., 310 aa, 311 aa, 312 aa, 313 aa, 314 aa, 315 aa, 316 aa, 317 aa, 318 aa, 319 aa, or 320 aa. In some cases, the Notch receptor polypeptide present in a chimeric polypeptide of the present disclosure has a length of 315 aa. In some cases, the Notch receptor polypeptide present in a chimeric polypeptide of the present disclosure has a length of from 360 aa to 370 aa, e.g., 360 aa, 361 aa, 362 aa, 363 aa 364 aa, 365 aa, 366 aa, 367 aa, 368 aa, 369 aa, or 370 aa. In some cases, the Notch receptor polypeptide present in a chimeric polypeptide of the present disclosure has a length of 367 aa.

In some cases, a Notch receptor polypeptide comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a Notch receptor including e.g., any of SEQ ID NOs: 1, 2 and 5-15. In some instances, the Notch regulatory region of a Notch receptor polypeptide is a mammalian Notch regulatory region, including but not limited to e.g., a mouse Notch (e.g., mouse Notch1, mouse Notch2, mouse Notch3 or mouse Notch4) regulatory region, a rat Notch regulatory region (e.g., rat Notch1, rat Notch2 or rat Notch3), a human Notch regulatory region (e.g., human Notch1, human Notch2, human Notch3 or human Notch4), and the like or a Notch regulatory region derived from a mammalian Notch regulatory region and having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of a mammalian Notch regulatory region of a mammalian Notch receptor amino acid sequence, including e.g., SEQ ID NOs: 1, 2 and 5-15.

Subject Notch regulatory regions may include or exclude various components (e.g., domains, cleavage sites, etc.) thereof. Examples of such components of Notch regulatory regions that may be present or absent in whole or in part, as appropriate, include e.g., one or more EGF-like repeat domains, one or more Lin12/Notch repeat domains, one or more heterodimerization domains (e.g., HD-N or HD-C), a transmembrane domain, one or more proteolytic cleavage sites (e.g., a furin-like protease site (e.g., an S1 site), an ADAM-family protease site (e.g., an S2 site) and/or a gamma-secretase protease site (e.g., an S3 site)), and the like. Notch receptor polypeptides may, in some instances, exclude all or a portion of one or more Notch extracellular domains, including e.g., Notch-ligand binding domains such as Delta-binding domains. Notch receptor polypeptides may, in some instances, include one or more non-functional versions of one or more Notch extracellular domains, including e.g., Notch-ligand binding domains such as Delta-binding domains. Notch receptor polypeptides may, in some instances, exclude all or a portion of one or more Notch intracellular domains, including e.g., Notch Rbp-associated molecule domains (i.e., RAM domains), Notch Ankyrin repeat domains, Notch transactivation domains, Notch PEST domains, and the like. Notch receptor polypeptides may, in some instances, include one or more non-functional versions of one or more Notch intracellular domains, including e.g., non-functional Notch Rbp-associated molecule domains (i.e., RAM domains), non-functional Notch Ankyrin repeat domains, non-functional Notch transactivation domains, non-functional Notch PEST domains, and the like.

### Notch Receptor Polypeptide Comprising a TM Domain

In some cases, the Notch receptor polypeptide present in a chimeric polypeptide of the present disclosure comprises an amino acid sequence having at least 75%, at least 80%, at least 85%, at least 90%, at least 95%, at least 98%, at least 99%, or 100%, amino acid sequence identity to the following amino acid sequence:
IPYKIEAVKSEPVEPPLPSQLHLMYVAAAAFVLLFFVGCGVLLSRKRRRQLCIQKL (SEQ ID NO: 89); where the TM domain is underlined; where the Notch receptor polypeptide comprises an S2 proteolytic cleavage site and an S3 proteolytic cleavage site; where the Notch receptor polypeptide has a length of from 50 amino acids (aa) to 65 aa, e.g., 50, 51, 52, 53, 54, 55, 56, 57, 58, 59, 60, 61, 62, 63, 64, or 65 aa. In some cases, the Notch receptor polypeptide present in a chimeric polypeptide of the present disclosure comprises an amino acid sequence having at least 75%, at least 80%, at least 85%, at least 90%, at least 95%, at least 98%, at least 99%, or 100%, amino acid sequence identity to the following amino acid sequence:
IPYKIEAVKSEPVEPPLPSQLHLMYVAAAAFVLLFFVGCGVLLSRKRRRQLCIQKL (SEQ ID NO: 89); where the TM domain is underlined; where the Notch receptor polypeptide comprises an S2 proteolytic cleavage site and an S3 proteolytic cleavage site; where the Notch receptor polypeptide has a length of 56 amino acids.

### Notch Receptor Polypeptide Comprising an LNR Segment, an HD-N Segment, an HD-C Segment, and a TM Domain

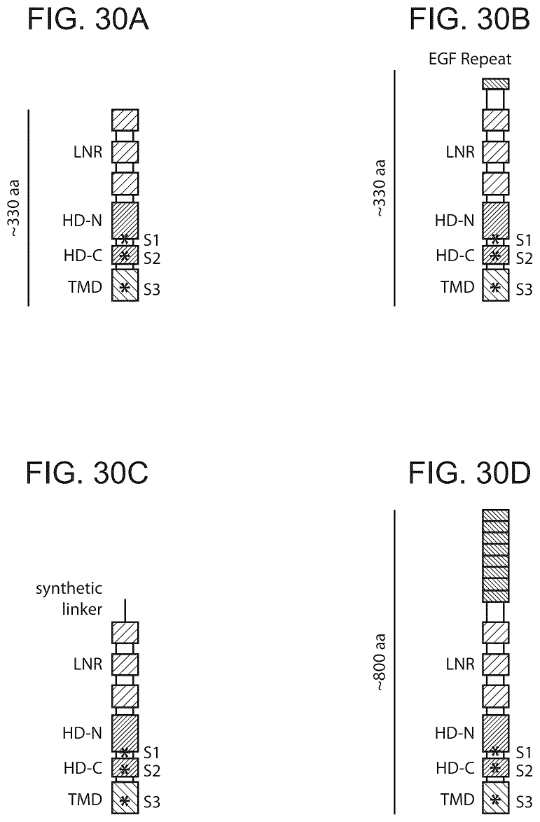

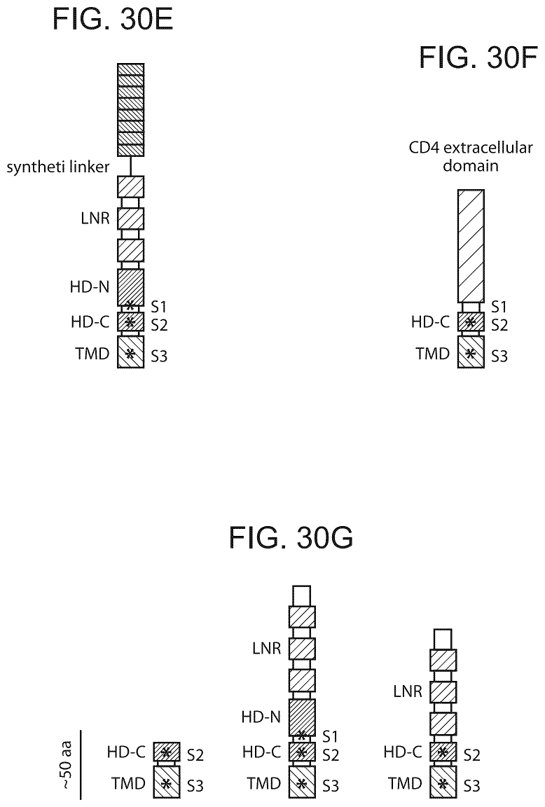

In some cases, the Notch receptor polypeptide present in a chimeric polypeptide of the present disclosure comprises, in order from N-terminus to C-terminus: i) a LNR-A segment; ii) a LNR-B segment; iii) a LNR-C segment; iv) an HD-N segment, v) an HD-C segment; and vi) a TM domain. A LNR-A segment, LNR-B segment, and LNR-C segment can collectively be referred to as an “LNR segment.” Such a Notch receptor polypeptide is depicted schematically in FIG. 30A.

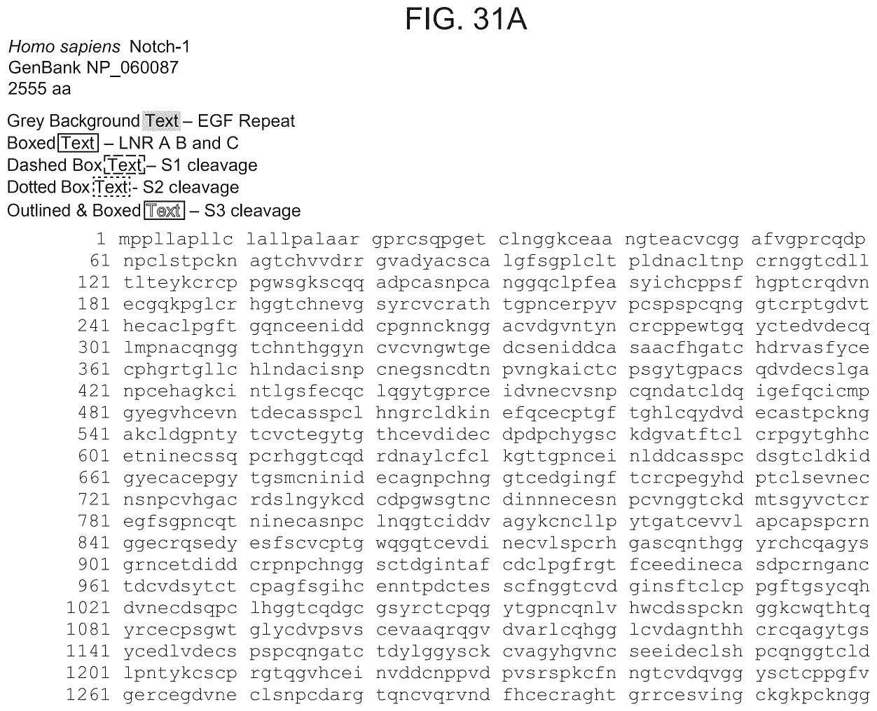

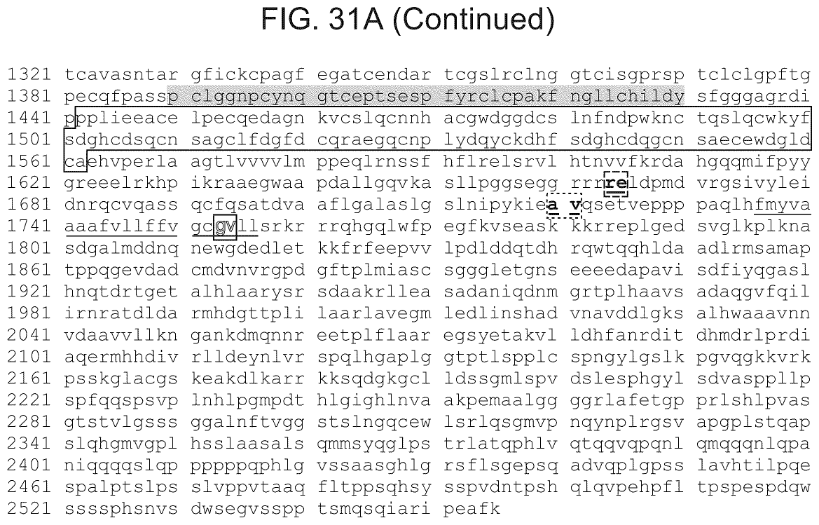

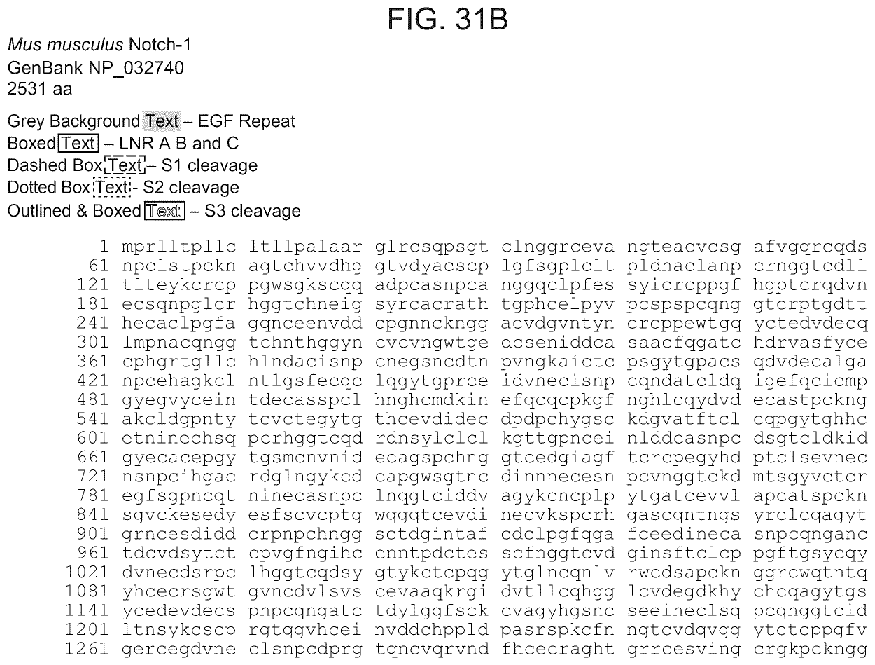

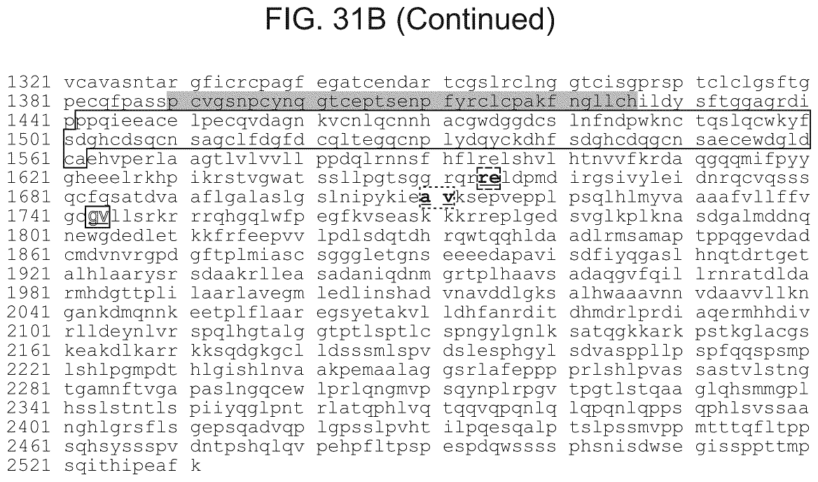

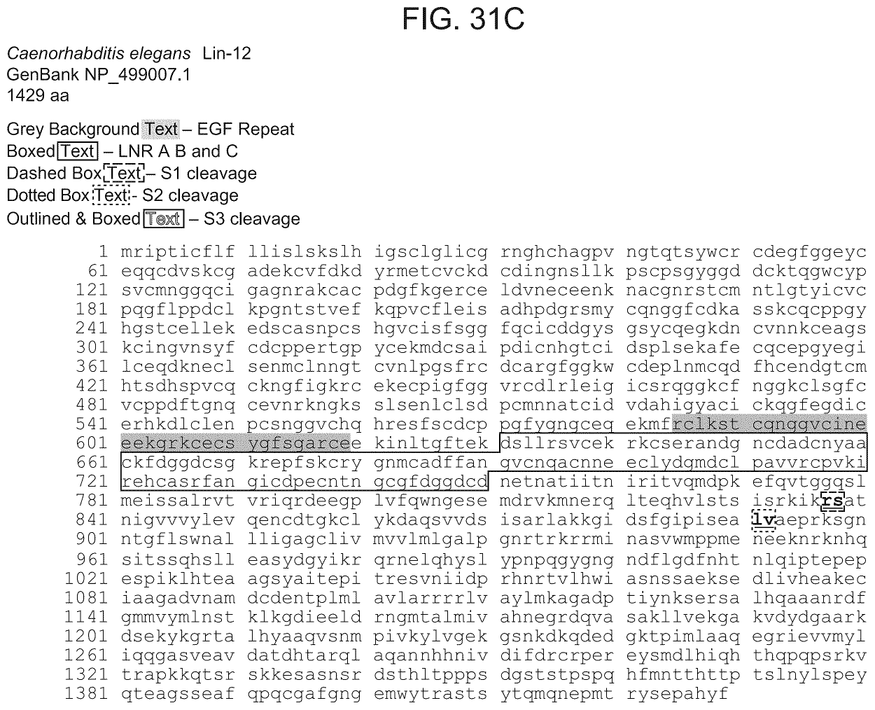

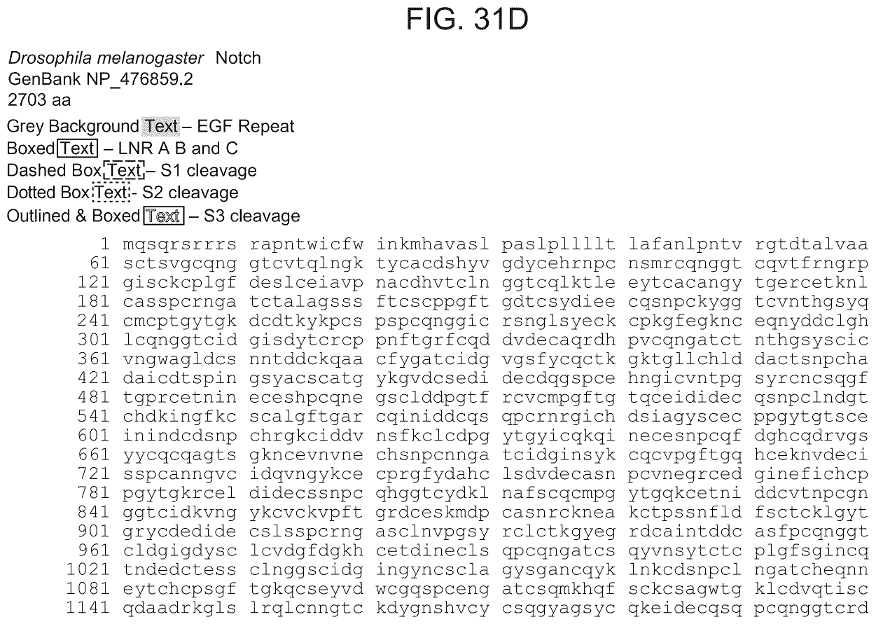

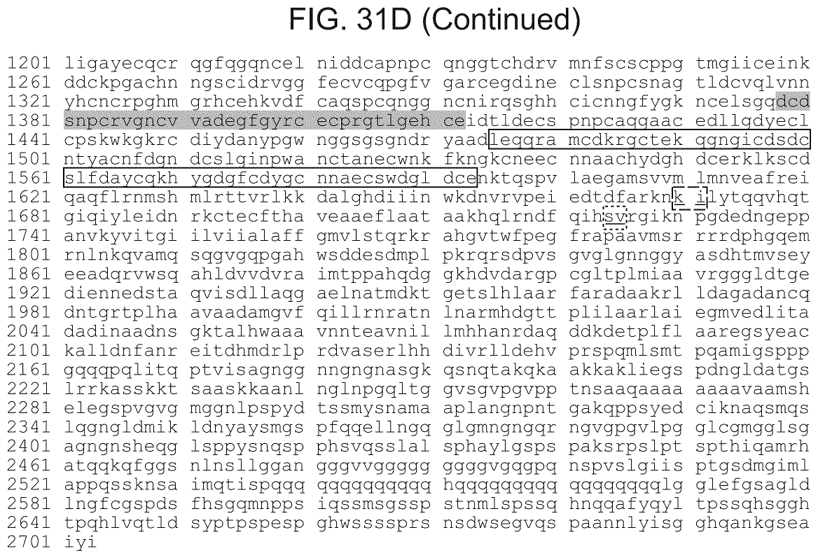

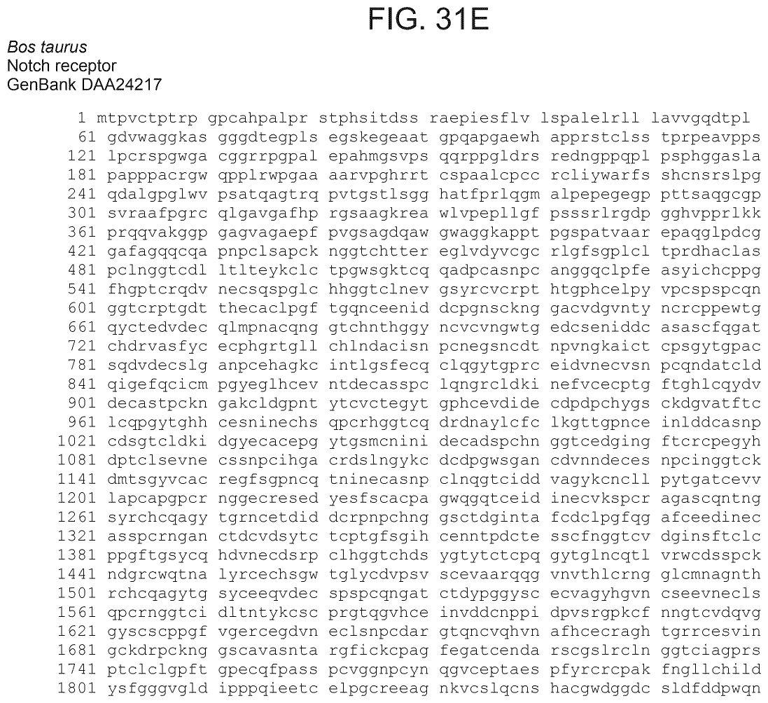

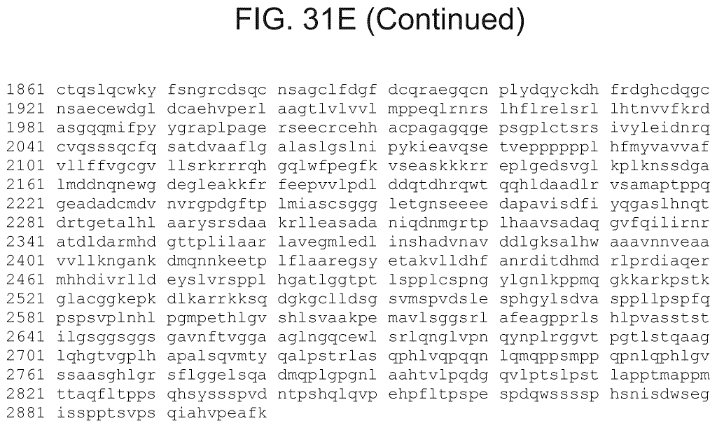

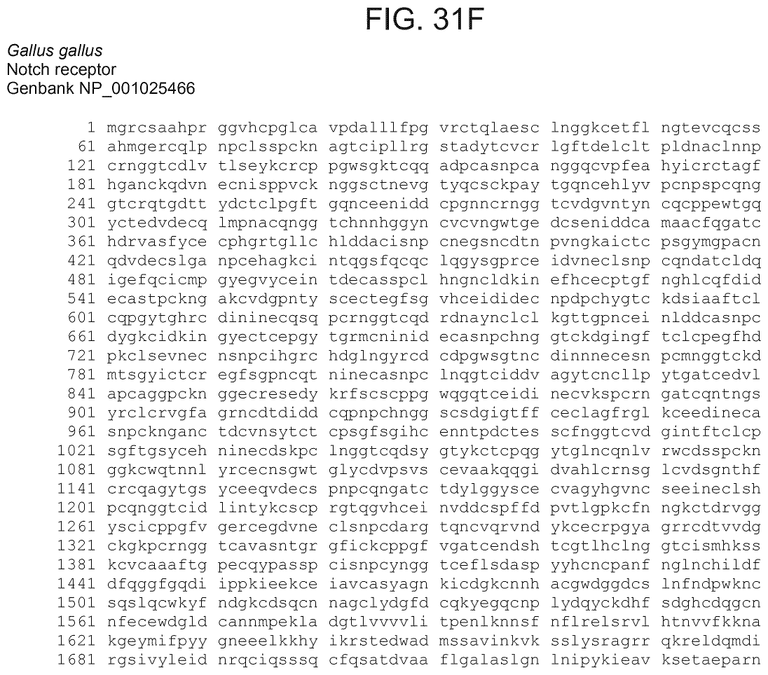

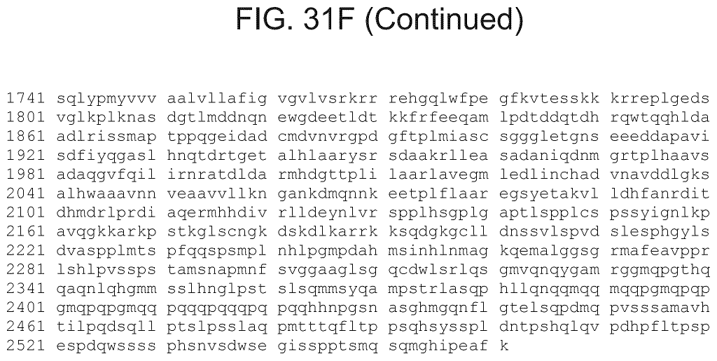

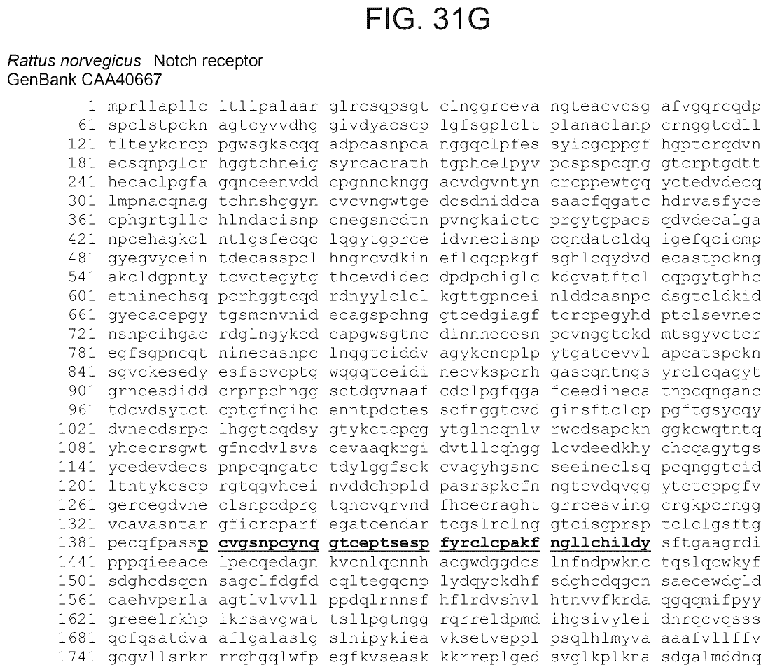

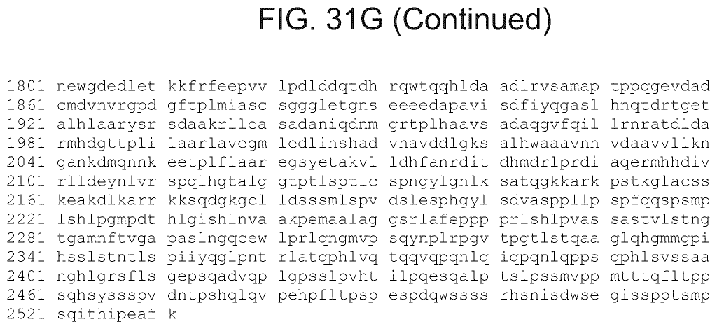

An LNR segment can comprise an amino acid sequence having at least 75%, at least 80%, at least 85%, at least 90%, at least 95%, at least 98%, at least 99%, or 100%, amino acid sequence identity to amino acids 1442-1562 of the amino acid sequence depicted in FIG. 31A, or a corresponding segment of another Notch receptor polypeptide, where examples of corresponding segments are depicted in FIG. 31B-31G; and can have a length of from 90 amino acids to 150 amino acids, e.g., from 90 amino acids (aa) to 100 aa, from 100 aa to 110 aa, from 110 aa to 120 aa, from 120 aa to 130 aa, from 130 aa to 140 aa, or from 140 aa to 150 aa. In some cases, an LNR segment comprises an amino acid sequence having at least 75%, at least 80%, at least 85%, at least 90%, at least 95%, at least 98%, at least 99%, or 100%, amino acid sequence identity to amino acids 1442-1562 of the amino acid sequence depicted in FIG. 31A, or a corresponding segment of another Notch receptor polypeptide, where examples of corresponding segments are depicted in FIG. 31B-31G; and has a length of from 115 aa to 125 aa, e.g., 115, 116, 117, 118, 119, 120, 121, 122, 123, 124, or 125 aa.

An LNR segment can comprise an amino acid sequence having at least 75%, at least 80%, at least 85%, at least 90%, at least 95%, at least 98%, at least 99%, or 100%, amino acid sequence identity to the following amino acid sequence:
PPQIEEACELPECQVDAGNKVCNLQCNNHACGWDGGDCSLNFNDPWKNCTQSLQCWKYFSDG HCDSQCNSAGCLFDGFDCQLTEGQCNPLYDQYCKDHFSDGHCDQGCNSAECEWDGLDC (SEQ ID NO: 90); and can have a length of from 118 to 122 amino acids (e.g., 118, 119, 120, 121, or 122 amino acids).

An HD-N segment can comprise an amino acid sequence having at least 75%, at least 80%, at least 85%, at least 90%, at least 95%, at least 98%, at least 99%, or 100%, amino acid sequence identity to amino acids 1563-1664 of the amino acid sequence depicted in FIG. 31A, or a corresponding segment of another Notch receptor polypeptide, where examples of corresponding segments are depicted in FIG. 31B-31G; and can have a length of from 90 amino acids (aa) to 110 aa, e.g., 90 aa to 95 aa, 95 aa to 100 aa, 100 aa to 105 aa, or 105 aa to 110 aa. In some cases, an HD-N segment comprises an amino acid sequence having at least 75%, at least 80%, at least 85%, at least 90%, at least 95%, at least 98%, at least 99%, or 100%, amino acid sequence identity to amino acids 1563-1664 of the amino acid sequence depicted in FIG. 31A, or a corresponding segment of another Notch receptor polypeptide, where examples of corresponding segments are depicted in FIG. 31B-31G; and has a length of from 95 aa to 105 aa, e.g., 95, 96, 98, 98, 99, 100, 101, 102, 103, 104, or 105 aa.

An HD-C segment can comprise an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and can have a length of from 60 amino acids (aa) to 80 aa, e.g., from 60 aa to 65 aa, from 65 aa to 70 aa, from 70 aa to 75 aa, or from 75 aa to 80 aa. In some cases, an HD-C segment comprises an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and has a length of from 65 amino acids to 75 amino acids, e.g., 65, 66, 67, 68, 69, 70, 71, 72, 73, 74, or 75 amino acids.

An HD segment (HD-N plus HD-C) can comprise an amino acid sequence having at least 75%, at least 80%, at least 85%, at least 90%, at least 95%, at least 98%, at least 99%, or 100%, amino acid sequence identity to the following amino acid sequence:
AAGTLVLVVLLPPDQLRNNSFHFLRELSHVLHTNVVFKRDAQGQQMIFPYYGHEEELRKHPIKR STVGWATSSLLPGTSGG [symbol] ELDPMDIRGSIVYLEIDNRQCVQSSSQCFQSATDVAAFLGALA SLGSLNIPYKIE [symbol] KSEPVEPPLP (SEQ ID NO:91); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence depicted in FIG. 31A, or a corresponding segment of another Notch receptor polypeptide, where examples of corresponding segments are depicted in FIG. 31B-31G; and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCG [symbol] (SEQ ID NO:92); and can have a length of 21, 22, 23, 24, or 25 amino acids.

In some cases, a Notch receptor polypeptide has a length of from about 310 amino acids (aa) to about 320 aa (e.g., 310 aa, 311 aa, 312 aa, 313 aa, 314 aa, 315 aa, 316 aa, 317 aa, 318 aa, 319 aa, or 320 aa), and comprises an amino acid sequence having at least 75%, at least 80%, at least 85%, at least 90%, at least 95%, at least 98%, at least 99%, or 100%, amino acid sequence identity to amino acids 1442-1756 of the amino acid sequence depicted in FIG. 31A, or a corresponding segment of another Notch receptor polypeptide, where examples of corresponding segments are depicted in FIG. 31B-31G.

In some cases, a Notch receptor polypeptide comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:
PPQIEEACELPECQVDAGNKVCNLQCNNHACGWDGGDCSLNFNDPWKNCTQSLQCWKYFSDG HCDSQCNSAGCLFDGFDCQLTEGQCNPLYDQYCKDHFSDGHCDQGCNSAECEWDGLDCAEHV PERLAAGTLVLVVLLPPDQLRNNSFHFLRELSHVLHTNVVFKRDAQGQQMIFPYYGHEEELRKH PIKRSTVGWATSSLLPGTSGGRQRRELDPMDIRGSIVYLEIDNRQCVQSSSQCFQSATDVAAFLG ALASLGSLNIPYKIEAVKSEPVEPPLPSQLHLMYVAAAAFVLLFFVGCGVLLS (SEQ ID NO:93);
and has a length of from 300 amino acids to 310 amino acids (e.g., 300, 301, 302, 303, 304, 305, 306, 307, 308, 309, or 310 amino acids).

In some cases, a Notch receptor polypeptide comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the following amino acid sequence:
PCVGSNPCYNQGTCEPTSENPFYRCLCPAKFNGLLCHILDYSFTGGAGRDIPPPQIEEACELPECQ VDAGNKVCNLQCNNHACGWDGGDCSLNFNDPWKNCTQSLQCWKYFSDGHCDSQCNSAGCLF DGFDCQLTEGQCNPLYDQYCKDHFSDGHCDQGCNSAECEWDGLDCAEHVPERLAAGTLVLVV LLPPDQLRNNSFHFLRELSHVLHTNVVFKRDAQGQQMIFPYYGHEEELRKHPIKRSTVGWATSS LLPGTSGGRQRRELDPMDIRGSIVYLEIDNRQCVQSSSQCFQSATDVAAFLGALASLGSLNIPYKI EAVKSEPVEPPLPSQLHLMYVAAAAFVLLFFVGCGVLLS (SEQ ID NO:94); and has a length of from 350 amino acids to 370 amino acids (e.g., 350 351, 352, 353, 354, 355, 356, 357, 358, 359, 360, 361, 362, 363, 364, 365, 366, 367, 368, 369, or 370 amino acids).

### Notch Receptor Polypeptide Comprising a Single EGF Repeat, an LNR Segment, an HD-N Segment, an HD-C Segment, and a TM Domain

In some cases, the Notch receptor polypeptide present in a chimeric polypeptide of the present disclosure comprises, in order from N-terminus to C-terminus: i) a single EGF repeat; ii) an LNR segment; iii) an HD-N segment, iv) an HD-C segment; and v) a TM domain. Such a Notch receptor polypeptide is depicted schematically in FIG. 30B.

An EGF repeat can comprises an amino acid sequence having at least 75%, at least 80%, at least 85%, at least 90%, at least 95%, at least 98%, at least 99%, or 100%, amino acid sequence identity to amino acids 1390 to 1430 of the amino acid sequence depicted in FIG. 31A, or a corresponding segment of another Notch receptor polypeptide, where examples of corresponding segments are depicted in FIG. 31B-31G; and can have a length of from 35 amino acids (aa) to 45 aa (e.g., 35, 36, 37, 38, 39, 40, 41, 42, 43, 44, or 45 aa).

An EGF repeat can comprise an amino acid sequence having at least 75%, at least 80%, at least 85%, at least 90%, at least 95%, at least 98%, at least 99%, or 100%, amino acid sequence identity to the following sequence: PCVGSNPCYNQGTCEPTSENPFYRCLCPAKFNGLLCH (SEQ ID NO:95); and can have a length of 35 amino acids to 40 amino acids (e.g., 35, 36, 37, 38, 39, or 40 amino acids).

An LNR segment can comprise an amino acid sequence having at least 75%, at least 80%, at least 85%, at least 90%, at least 95%, at least 98%, at least 99%, or 100%, amino acid sequence identity to amino acids 1442-1562 of the amino acid sequence depicted in FIG. 31A, or a corresponding segment of another Notch receptor polypeptide, where examples of corresponding segments are depicted in FIG. 31B-31G; and can have a length of from 90 amino acids to 150 amino acids, e.g., from 90 amino acids (aa) to 100 aa, from 100 aa to 110 aa, from 110 aa to 120 aa, from 120 aa to 130 aa, from 130 aa to 140 aa, or from 140 aa to 150 aa. In some cases, an LNR segment comprises an amino acid sequence having at least 75%, at least 80%, at least 85%, at least 90%, at least 95%, at least 98%, at least 99%, or 100%, amino acid sequence identity to amino acids 1442-1562 of the amino acid sequence depicted in FIG. 31A, or a corresponding segment of another Notch receptor polypeptide, where examples of corresponding segments are depicted in FIG. 31B-31G; and has a length of from 115 aa to 125 aa, e.g., 115, 116, 117, 118, 119, 120, 121, 122, 123, 124, or 125 aa.

An LNR segment can comprise an amino acid sequence having at least 75%, at least 80%, at least 85%, at least 90%, at least 95%, at least 98%, at least 99%, or 100%, amino acid sequence identity to the following amino acid sequence:
PPQIEEACELPECQVDAGNKVCNLQCNNHACGWDGGDCSLNFNDPWKNCTQSLQCWKYFSDG HCDSQCNSAGCLFDGFDCQLTEGQCNPLYDQYCKDHFSDGHCDQGCNSAECEWDGLDC (SEQ ID NO: 90); and can have a length of from 118 to 122 amino acids (e.g., 118, 119, 120, 121, or 122 amino acids).

An HD-N segment can comprise an amino acid sequence having at least 75%, at least 80%, at least 85%, at least 90%, at least 95%, at least 98%, at least 99%, or 100%, amino acid sequence identity to amino acids 1563-1664 of the amino acid sequence depicted in FIG. 31A, or a corresponding segment of another Notch receptor polypeptide, where examples of corresponding segments are depicted in FIG. 31B-31G; and can have a length of from 90 amino acids (aa) to 110 aa, e.g., 90 aa to 95 aa, 95 aa to 100 aa, 100 aa to 105 aa, or 105 aa to 110 aa. In some cases, an HD-N segment comprises an amino acid sequence having at least 75%, at least 80%, at least 85%, at least 90%, at least 95%, at least 98%, at least 99%, or 100%, amino acid sequence identity to amino acids 1563-1664 of the amino acid sequence depicted in FIG. 31A, or a corresponding segment of another Notch receptor polypeptide, where examples of corresponding segments are depicted in FIG. 31B-31G; and has a length of from 95 aa to 105 aa, e.g., 95, 96, 98, 98, 99, 100, 101, 102, 103, 104, or 105 aa.

An HD-C segment can comprise an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and can have a length of from 60 amino acids (aa) to 80 aa, e.g., from 60 aa to 65 aa, from 65 aa to 70 aa, from 70 aa to 75 aa, or from 75 aa to 80 aa. In some cases, an HD-C segment comprises an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and has a length of from 65 amino acids to 75 amino acids, e.g., 65, 66, 67, 68, 69, 70, 71, 72, 73, 74, or 75 amino acids.

An HD segment (HD-N plus HD-C) can comprise an amino acid sequence having at least 75%, at least 80%, at least 85%, at least 90%, at least 95%, at least 98%, at least 99%, or 100%, amino acid sequence identity to the following amino acid sequence:
AAGTLVLVVLLPPDQLRNNSFHFLRELSHVLHTNVVFKRDAQGQQMIFPYYGHEEELRKHPIKR STVGWATSSLLPGTSGG [symbol] ELDPMDIRGSIVYLEIDNRQCVQSSSQCFQSATDVAAFLGALA SLGSLNIPYKIE [symbol] KSEPVEPPLP (SEQ ID NO:91); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence depicted in FIG. 31A, or a corresponding segment of another Notch receptor polypeptide, where examples of corresponding segments are depicted in FIG. 31B-31G; and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCG [symbol] (SEQ ID NO:92); and can have a length of 21, 22, 23, 24, or 25 amino acids.

In some cases, a Notch receptor polypeptide has a length of from about 360 amino acids (aa) to about 375 aa (e.g., 360 aa, 361 aa, 362 aa, 363 aa, 364 aa, 365 aa, 366 aa, 367 aa, 368 aa, 369 aa, 370 aa, 371 aa, 372 aa, 373 aa, 374 aa, or 375 aa), and comprises an amino acid sequence having at least 75%, at least 80%, at least 85%, at least 90%, at least 95%, at least 98%, at least 99%, or 100%, amino acid sequence identity to amino acids 1390-1756 of the amino acid sequence depicted in FIG. 31A, or a corresponding segment of another Notch receptor polypeptide, where examples of corresponding segments are depicted in FIG. 31B-31G.

In some cases, a Notch receptor polypeptide comprises a synthetic linker. For example, in some cases, a Notch receptor polypeptide comprises, in order from N-terminus to C-terminus: i) a synthetic linker; ii) an EGF repeat; iii) an LNR segment; iv) an HD-N segment, v) an HD-C segment; and vi) a TM domain. Such a Notch receptor polypeptide is depicted schematically in FIG. 30C.

A synthetic linker can have a length of from about 10 amino acids (aa) to about 200 aa, e.g., from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A synthetic linker can have a length of from 10 aa to 30 aa, e.g., 10, 11, 12, 13, 14, 15, 16, 17, 18, 19, 20, 21, 22, 23, 24, 25, 26, 27, 28, 29, or 30 aa. A synthetic linker can have a length of from 30 aa to 50 aa, e.g., from 30 aa to 35 aa, from 35 aa to 40 aa, from 40 aa to 45 aa, or from 45 aa to 50 aa.

In some instances, a synthetic linker, as described herein, may include an extracellular protein structural domain or a portion thereof. Extracellular protein structural domains suitable for use as a synthetic linker include but are not limited to e.g., Ig-like extracellular structural domains, Fc extracellular structural domains, fibronectin extracellular structural domains and the like. In some instances, a synthetic linker may include a plurality of extracellular protein structural domains where the plurality may include a plurality of the same domain or a plurality of different domains.

### Notch Receptor Polypeptide Comprising 2-11 EGF Repeats, an LNR Segment, an HD-N Segment, an HD-C Segment, and a TM Domain

In some cases, the Notch receptor polypeptide present in a chimeric polypeptide of the present disclosure comprises, in order from N-terminus to C-terminus: i) from two to eleven EGF repeats; ii) an LNR segment; iii) an HD-N segment, iv) an HD-C segment; and v) a TM domain. Such a Notch receptor polypeptide is depicted schematically in FIG. 30D.

In some cases, the Notch receptor polypeptide present in a chimeric polypeptide of the present disclosure comprises, in order from N-terminus to C-terminus: i) two EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) three EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) four EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) five EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) six EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) seven EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) eight EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) nine EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) ten EGF repeats; ii) an LNR segment; iii) an HD-N segment, iv) an HD-C segment; and v) a TM domain. In some cases, the Notch receptor polypeptide present in a chimeric polypeptide of the present disclosure comprises, in order from N-terminus to C-terminus: i) eleven EGF repeats; ii) an LNR segment; iii) an HD-N segment, iv) an HD-C segment; and v) a TM domain.

An EGF repeat can comprise an amino acid sequence having at least 75%, at least 80%, at least 85%, at least 90%, at least 95%, at least 98%, at least 99%, or 100%, amino acid sequence identity to amino acids 1390 to 1430 of the amino acid sequence depicted in FIG. 31A, or a corresponding segment of another Notch receptor polypeptide, where examples of corresponding segments are depicted in FIG. 31B-31G; and can have a length of from 35 amino acids (aa) to 45 aa (e.g., 35, 36, 37, 38, 39, 40, 41, 42, 43, 44, or 45 aa).

An EGF repeat can comprise an amino acid sequence having at least 75%, at least 80%, at least 85%, at least 90%, at least 95%, at least 98%, at least 99%, or 100%, amino acid sequence identity to amino acids 869-905 (DINECVLSPCRHGASCQNTHGGYRCHCQAGYSGRNCE; SEQ ID NO:96) of the amino acid sequence depicted in FIG. 31A, or a corresponding segment of another Notch receptor polypeptide, where examples of corresponding segments are depicted in FIG. 31B-31G; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 907-943 (DIDDCRPNPCHNGGSCTDGINTAFCDCLPGFRGTFCE; SEQ ID NO:97) of the amino acid sequence depicted in FIG. 31A, or a corresponding segment of another Notch receptor polypeptide, where examples of corresponding segments are depicted in FIG. 31B-31G; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 945-981 (DINECASDPCRNGANCTDCVDSYTCTCPAGFSGIHCE; (SEQ ID NO:98) of the amino acid sequence depicted in FIG. 31A, or a corresponding segment of another Notch receptor polypeptide, where examples of corresponding segments are depicted in FIG. 31B-31G; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 988-1019 (TESSCFNGGTCVDGINSFTCLCPPGFTGSYCQ; SEQ ID NO:99) of the amino acid sequence depicted in FIG. 31A, or a corresponding segment of another Notch receptor polypeptide, where examples of corresponding segments are depicted in FIG. 31B-31G; and can have a length of from 30 amino acids (aa) to 35 aa (e.g., 30, 31, 32, 33, 34, or 35 aa).

An EGF repeat can comprise an amino acid sequence having at least 75%, at least 80%, at least 85%, at least 90%, at least 95%, at least 98%, at least 99%, or 100%, amino acid sequence identity to amino acids 1021-1057 (DVNECDSQPCLHGGTCQDGCGSYRCTCPQGYTGPNCQ; SEQ ID NO: 100) of the amino acid sequence depicted in FIG. 31A, or a corresponding segment of another Notch receptor polypeptide, where examples of corresponding segments are depicted in FIG. 31B-31G; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 1064-1090 (DSSPCKNGGKCWQTHTQYRCECPSGWT; SEQ ID NO:101) of the amino acid sequence depicted in FIG. 31A, or a corresponding segment of another Notch receptor polypeptide, where examples of corresponding segments are depicted in FIG. 31B-31G; and can have a length of from 25 amino acids (aa) to 30 aa, e.g., 25, 26, 27, 28, 29, or 30 aa.

An EGF repeat can comprise an amino acid sequence having at least 75%, at least 80%, at least 85%, at least 90%, at least 95%, at least 98%, at least 99%, or 100%, amino acid sequence identity to amino acids 1146-1180 (LVDECSPSPCQNGATCTDYLGGYSCKCVAGYHGVNC; SEQ ID NO:102) of the amino acid sequence depicted in FIG. 31A, or a corresponding segment of another Notch receptor polypeptide, where examples of corresponding segments are depicted in FIG. 31B-31G; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 1184-1219 (IDECLSHPCQNGGTCLDLPNTYKCSCPRGTQGVHCE; SEQ ID NO:103) of the amino acid sequence depicted in FIG. 31A, or a corresponding segment of another Notch receptor polypeptide, where examples of corresponding segments are depicted in FIG. 31B-31G; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to amino acids 1238-1265 (CFNNGTCVDQVGGYSCTCPPGFVGERCE; SEQ ID NO:104) of the amino acid sequence depicted in FIG. 31A, or a corresponding segment of another Notch receptor polypeptide, where examples of corresponding segments are depicted in FIG. 31B-31G; and can have a length of from 25 amino acids (aa) to 30 aa, e.g., 25, 26, 27, 28, 29, or 30 aa.

An EGF repeat can comprise an amino acid sequence having at least 75%, at least 80%, at least 85%, at least 90%, at least 95%, at least 98%, at least 99%, or 100%, amino acid sequence identity to amino acids 1267-1305 (DVNECLSNPCDARGTQNCVQRVNDFHCECRAGHTGRRCE; (SEQ ID NO: 105) of the amino acid sequence depicted in FIG. 31A, or a corresponding segment of another Notch receptor polypeptide, where examples of corresponding segments are depicted in FIG. 31B-31G; and can have a length of from 35 amino acids to about 40 amino acids (aa) (e.g., 35, 36, 37, 38, 39, or 40 aa).

An EGF repeat can comprise an amino acid sequence having at least 75%, at least 80%, at least 85%, at least 90%, at least 95%, at least 98%, at least 99%, or 100%, amino acid sequence identity to the following sequence: PCVGSNPCYNQGTCEPTSENPFYRCLCPAKFNGLLCH (SEQ ID NO:95); and can have a length of 35 amino acids to 40 amino acids (e.g., 35, 36, 37, 38, 39, or 40 amino acids.

An LNR segment can comprise an amino acid sequence having at least 75%, at least 80%, at least 85%, at least 90%, at least 95%, at least 98%, at least 99%, or 100%, amino acid sequence identity to amino acids 1442-1562 of the amino acid sequence depicted in FIG. 31A, or a corresponding segment of another Notch receptor polypeptide, where examples of corresponding segments are depicted in FIG. 31B-31G; and can have a length of from 90 amino acids to 150 amino acids, e.g., from 90 amino acids (aa) to 100 aa, from 100 aa to 110 aa, from 110 aa to 120 aa, from 120 aa to 130 aa, from 130 aa to 140 aa, or from 140 aa to 150 aa. In some cases, an LNR segment comprises an amino acid sequence having at least 75%, at least 80%, at least 85%, at least 90%, at least 95%, at least 98%, at least 99%, or 100%, amino acid sequence identity to amino acids 1442-1562 of the amino acid sequence depicted in FIG. 31A, or a corresponding segment of another Notch receptor polypeptide, where examples of corresponding segments are depicted in FIG. 31B-31G; and has a length of from 115 aa to 125 aa, e.g., 115, 116, 117, 118, 119, 120, 121, 122, 123, 124, or 125 aa.

An LNR segment can comprise an amino acid sequence having at least 75%, at least 80%, at least 85%, at least 90%, at least 95%, at least 98%, at least 99%, or 100%, amino acid sequence identity to the following amino acid sequence:
PPQIEEACELPECQVDAGNKVCNLQCNNHACGWDGGDCSLNFNDPWKNCTQSLQCWKYFSDG HCDSQCNSAGCLFDGFDCQLTEGQCNPLYDQYCKDHFSDGHCDQGCNSAECEWDGLDC (SEQ ID NO: 90); and can have a length of from 118 to 122 amino acids (e.g., 118, 119, 120, 121, or 122 amino acids).

An HD-N segment can comprise an amino acid sequence having at least 75%, at least 80%, at least 85%, at least 90%, at least 95%, at least 98%, at least 99%, or 100%, amino acid sequence identity to amino acids 1563-1664 of the amino acid sequence depicted in FIG. 31A, or a corresponding segment of another Notch receptor polypeptide, where examples of corresponding segments are depicted in FIG. 31B-31G; and can have a length of from 90 amino acids (aa) to 110 aa, e.g., 90 aa to 95 aa, 95 aa to 100 aa, 100 aa to 105 aa, or 105 aa to 110 aa. In some cases, an HD-N segment comprises an amino acid sequence having at least 75%, at least 80%, at least 85%, at least 90%, at least 95%, at least 98%, at least 99%, or 100%, amino acid sequence identity to amino acids 1563-1664 of the amino acid sequence depicted in FIG. 31A, or a corresponding segment of another Notch receptor polypeptide, where examples of corresponding segments are depicted in FIG. 31B-31G; and has a length of from 95 aa to 105 aa, e.g., 95, 96, 98, 98, 99, 100, 101, 102, 103, 104, or 105 aa.

An HD-C segment can comprise an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and can have a length of from 60 amino acids (aa) to 80 aa, e.g., from 60 aa to 65 aa, from 65 aa to 70 aa, from 70 aa to 75 aa, or from 75 aa to 80 aa. In some cases, an HD-C segment comprises an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and has a length of from 65 amino acids to 75 amino acids, e.g., 65, 66, 67, 68, 69, 70, 71, 72, 73, 74, or 75 amino acids.

An HD segment (HD-N plus HD-C) can comprise an amino acid sequence having at least 75%, at least 80%, at least 85%, at least 90%, at least 95%, at least 98%, at least 99%, or 100%, amino acid sequence identity to the following amino acid sequence:
AAGTLVLVVLLPPDQLRNNSFHFLRELSHVLHTNVVFKRDAQGQQMIFPYYGHEEELRKHPIKR STVGWATSSLLPGTSGG [symbol] ELDPMDIRGSIVYLEIDNRQCVQSSSQCFQSATDVAAFLGALA SLGSLNIPYKIE [symbol] KSEPVEPPLP (SEQ ID NO:91); and can have a length of 150, 151, 152, 153, or 154 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence depicted in FIG. 31A, or a corresponding segment of another Notch receptor polypeptide, where examples of corresponding segments are depicted in FIG. 31B-31G; and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCG [symbol] (SEQ ID NO:92); and can have a length of 21, 22, 23, 24, or 25 amino acids.

In some cases, a Notch receptor polypeptide has a length of from about 490 amino acids (aa) to about 900 aa, and comprises an amino acid sequence having at least 75%, at least 80%, at least 85%, at least 90%, at least 95%, at least 98%, at least 99%, or 100%, amino acid sequence identity to: i) amino acids 1267-1756; ii) 1238-1756; iii) 1184-1756; iv) 1146-1756; v) 1064-1756; vi) 1021-1756; vii) 988-1756; viii) 945-1756; ix) 907-1756; or x) 869-1756, of the amino acid sequence depicted in FIG. 31A, or a corresponding segment of another Notch receptor polypeptide, where examples of corresponding segments are depicted in FIG. 31B-31G.

In some cases, a Notch receptor polypeptide comprises a synthetic linker. For example, in some cases, a Notch receptor polypeptide comprises, in order from N-terminus to C-terminus: i) two to eleven EGF repeats; ii) a synthetic linker; iii) an LNR segment; iv) an HD-N segment, v) an HD-C segment; and vi) a TM domain. Such a Notch receptor polypeptide is depicted schematically in FIG. 30E.

A synthetic linker can have a length of from about 10 amino acids (aa) to about 200 aa, e.g., from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A synthetic linker can have a length of from 10 aa to 30 aa, e.g., 10, 11, 12, 13, 14, 15, 16, 17, 18, 19, 20, 21, 22, 23, 24, 25, 26, 27, 28, 29, or 30 aa. A synthetic linker can have a length of from 30 aa to 50 aa, e.g., from 30 aa to 35 aa, from 35 aa to 40 aa, from 40 aa to 45 aa, or from 45 aa to 50 aa.

### Notch Receptor Polypeptide Comprising an HD-C Segment and a TM Domain

In some cases, a Notch receptor polypeptide comprises, in order from N-terminus to C-terminus: i) an HD-C segment; and ii) a TM domain, where the Notch receptor polypeptide does not include an LNR segment. In some cases, the LNR segment is replaced with a heterologous polypeptide. Such a Notch receptor polypeptide is depicted schematically in FIG. 30F.

An HD-C segment can comprise an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and can have a length of from 60 amino acids (aa) to 80 aa, e.g., from 60 aa to 65 aa, from 65 aa to 70 aa, from 70 aa to 75 aa, or from 75 aa to 80 aa. In some cases, an HD-C segment comprises an amino acid sequence having at least 75%, at least 80%, at least 85%, at least 90%, at least 95%, at least 98%, at least 99%, or 100%, amino acid sequence identity to amino acids 1665-1733 of the amino acid sequence depicted in FIG. 31A, or a corresponding segment of another Notch receptor polypeptide, where examples of corresponding segments are depicted in FIG. 31B-31G; and has a length of from 65 amino acids to 75 amino acids, e.g., 65, 66, 67, 68, 69, 70, 71, 72, 73, 74, or 75 amino acids.

A transmembrane segment can comprise an amino acid sequence having at least 75%, at least 80%, at least 85%, at least 90%, at least 95%, at least 98%, at least 99%, or 100%, amino acid sequence identity to amino acids 1736 to 1756 of the amino acid sequence depicted in FIG. 31A, or a corresponding segment of another Notch receptor polypeptide, where examples of corresponding segments are depicted in FIG. 31B-31G; and can have a length of from 15 amino acids (aa) to 25 amino acids, e.g., 15, 16, 17, 18, 29, 20, 21, 22, 23, 24, or 25 amino acids.

A transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCG [symbol] (SEQ ID NO:92); and can have a length of 21, 22, 23, 24, or 25 amino acids.

In some cases, a Notch receptor polypeptide comprises an amino acid sequence having at least 75%, at least 80%, at least 85%, at least 90%, at least 95%, at least 98%, at least 99%, or 100%, amino acid sequence identity to amino acids 1665 to 1756 of the amino acid sequence depicted in FIG. 31A, or a corresponding segment of another Notch receptor polypeptide, where examples of corresponding segments are depicted in FIG. 31B-31G; and has a length of from 85 amino acids (aa) to 95 aa (e.g., 85, 86, 87, 88, 89, 90, 91, 92, 93, 94, or 95 aa).

In some cases, a Notch receptor polypeptide comprises an amino acid sequence having at least 75%, at least 80%, at least 85%, at least 90%, at least 95%, at least 98%, at least 99%, or 100%, amino acid sequence identity to amino acids 1665 to 1756 of the amino acid sequence depicted in FIG. 31A, or a corresponding segment of another Notch receptor polypeptide, where examples of corresponding segments are depicted in FIG. 31B-31G; and comprises a heterologous polypeptide fused in-frame at the N-terminus of the Notch receptor polypeptide.

### Proteolytic Cleavage Sites

As noted above, a chimeric polypeptide of the present disclosure comprises a Notch receptor polypeptide, e.g., having a length of from 50 amino acids to 1000 amino acids, and having one or more proteolytic cleavage sites.

In some cases, the Notch receptor polypeptide includes only one proteolytic cleavage site. In some cases, the Notch receptor polypeptide includes two proteolytic cleavage sites. In some cases, the Notch receptor polypeptide includes three proteolytic cleavage sites. For simplicity, cleavage sites will be referred to herein as “S1,” “S2,” and “S3” proteolytic cleavage sites.

In some cases, the Notch receptor polypeptide includes an S1 proteolytic cleavage site. An S1 proteolytic cleavage site can be located between the HD-N segment and the HD-C segment. In some cases, the S1 proteolytic cleavage site is a furin-like protease cleavage site. A furin-like protease cleavage site can have the canonical sequence Arg-X-(Arg/Lys)-Arg (SEQ ID NO:130), where X is any amino acid; the protease cleaves immediately C-terminal to the canonical sequence. For example, in some cases, an amino acid sequence comprising an S1 proteolytic cleavage site can have the amino acid sequence GRRRRELDPM (SEQ ID NO:106), where cleavage occurs between the “RE” sequence. As another example, an amino acid sequence comprising an S1 proteolytic cleavage site can have the amino acid sequence RQRRELDPM (SEQ ID NO:107), where cleavage occurs between the “RE” sequence.

In some cases, the Notch receptor polypeptide includes an S2 proteolytic cleavage site. An S2 proteolytic cleavage site can be located within the HD-C segment. In some cases, the S2 proteolytic cleavage site is an ADAM family type protease cleavage site, such as e.g., an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 proteolytic cleavage site can have the amino acid sequence KIEAVKSE (SEQ ID NO:108), where cleavage occurs between the “AV” sequence. As another example, an amino acid sequence comprising an S2 proteolytic cleavage site can have the amino acid sequence KIEAVQSE (SEQ ID NO:109), where cleavage occurs between the “AV” sequence.

In some cases, the Notch receptor polypeptide includes an S3 proteolytic cleavage site. An S3 proteolytic cleavage site can be located within the TM domain. In some cases, the S3 proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 proteolytic cleavage site has the amino acid sequence VGCGVLLS (SEQ ID NO: 110), where cleavage occurs between the “GV” sequence. In some cases, an S3 proteolytic cleavage site comprises the amino acid sequence GCGVLLS (SEQ ID NO:111).

In some cases, the Notch receptor polypeptide lacks an S1 proteolytic cleavage site. In some cases, the Notch receptor polypeptide lacks an S2 proteolytic cleavage site. In some cases, the Notch receptor polypeptide lacks an S3 proteolytic cleavage site. In some cases, the Notch receptor polypeptide lacks both an S1 proteolytic cleavage site and an S2 proteolytic cleavage site. In some cases, the Notch receptor polypeptide includes an S3 proteolytic cleavage site; and lacks both an S1 proteolytic cleavage site and an S2 proteolytic cleavage site. Examples are depicted schematically in FIG. 30G.

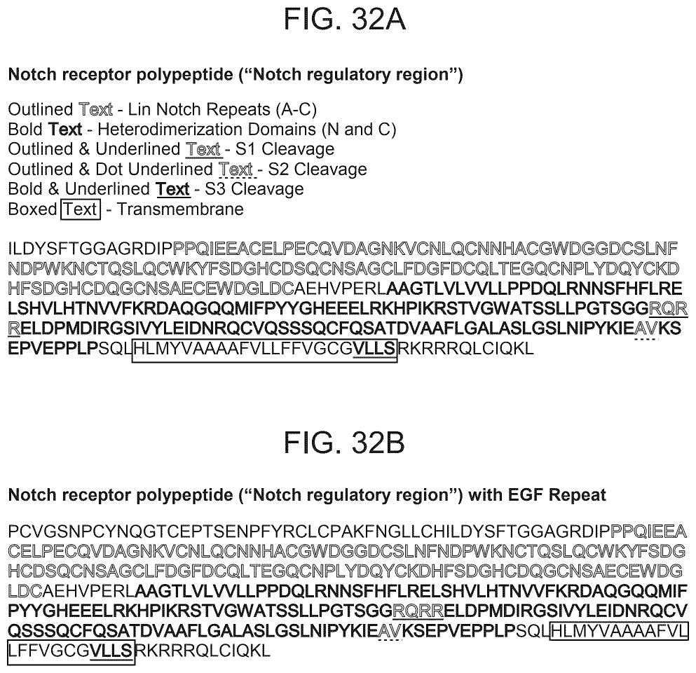

In some instances, a Notch receptor polypeptide of a chimeric polypeptide of the present disclosure may be a Notch receptor polypeptide of FIG. 32A-32B or may include all or none or more parts of a Notch receptor polypeptide presented in FIG. 32A-32B.

Certain Notch receptor polypeptides and components thereof that may be adapted for use in the chimeric polypeptides and the methods and circuits described herein include but are not limited to e.g., those described in PCT Application No. US2016/019188 (Pub. No. WO 2016/138034), the disclosure of which is incorporated herein by reference in its entirety.

### Intracellular Domains

As noted above, a chimeric polypeptide of the present disclosure comprises an intracellular domain that is released following binding of the chimeric polypeptide to the binding partner of the extracellular specific binding member, where such binding induces cleavage of an above-mentioned proteolytic cleavage site.

The intracellular domain comprises an amino acid sequence that is heterologous to the Notch receptor polypeptide. In other words, the intracellular domain comprises an amino acid sequence that is not naturally present in a Notch receptor polypeptide.

In some instances, the intracellular domain, when released from the chimeric polypeptide, induces a transcriptional response in the cell or otherwise modulates transcription. For example, in some instances, the intracellular domain activates transcription within the cell and thus serves as a transcriptional activator and may contain a transcription activation domain.

The intracellular domain may provide essentially any effector function attributable to an expressed peptide or protein, wherein such effector functions may include but are not limited to, e.g., increased production of one or more cytokines by the cell; reduced production of one or more cytokines by the cell; increased or decreased production of a hormone by the cell; production of an antibody by the cell; a change in organelle activity; a change in trafficking of a polypeptide within the cell; a change in transcription of a target gene; a change in activity of a protein; a change in cell activity, e.g., cell death; cellular proliferation; effects on cellular differentiation; effects on cell survival; modulation of cellular signaling responses; etc. In some cases, the intracellular domain, when released from the chimeric polypeptide, provides for a change in transcription of a target gene. In some cases, the intracellular domain, when released from the chimeric polypeptide, provides for an increase in the transcription of a target gene. In some cases, the intracellular domain, when released from the chimeric polypeptide, provides for a decrease in expression of a target gene.

In some instances, the intracellular domain of a proteolytically cleavable chimeric polypeptide of the instant disclosure includes a transcriptional activator. Any convenient transcriptional activator may find use in the intracellular domain of a chimeric polypeptide of the instant disclosure. Within a cell or system, a transcriptional activator may be paired with a transcriptional control element that is responsive to the transcriptional activator, e.g., to drive expression of a nucleic acid encoding a polypeptide of interest that is operably linked to the transcriptional control element. Useful transcriptional activators, transcriptional control elements, activator/control element pairs, and components of such systems may include but are not limited to e.g., those used in inducible expression systems including but not limited to e.g., those described in Goverdhana et al. Mol Ther. (2005) 12(2): 189-211; U.S. Patent Application Pub. Nos. 20160152701, 20150376627, 20130212722, 20070077642, 20050164237, 20050066376, 20040235169, 20040038249, 20030220286, 20030199022, 20020106720; the disclosures of which are incorporated herein by reference in their entirety.

In some instances, useful transcriptional activators may include mammalian transcription factors or engineered or mutated forms thereof. In some instances, useful transcriptional activators may include human transcription factors or engineered or mutated forms thereof. In some instances, useful transcriptional activators may include mouse transcription factors or engineered or mutated forms thereof. In some instances, useful transcriptional activators may include rat transcription factors or engineered or mutated forms thereof. In some instances, useful transcriptional activators may include cow transcription factors or engineered or mutated forms thereof. In some instances, useful transcriptional activators may include pig transcription factors or engineered or mutated forms thereof.

In some instances, use of a mammalian transcription factor may reduce the chance that the transcription factor induces an immune response in a mammal. In some instances, use of an engineered or mutated transcription factor, including e.g., mutated or engineered mammalian transcription factors, may reduce the chance that the transcription factor induces an immune response in a mammal. Useful mammalian transcription factors include but are not limited to e.g., zinc finger (ZnF) proteins.

In some instances, the intracellular domain is a transcriptional activator. In some cases, the intracellular domain comprises an amino acid sequence having at least 75%, at least 80%, at least 85%, at least 90%, at least 95%, at least 98%, at least 99%, or 100%, amino acid sequence identity to the following tetracycline-controlled transcriptional activator (tTA) amino acid sequence:
MSRLDKSKVINSALELLNEVGIEGLTTRKLAQKLGVEQPTLYWHVKNKRALLDALAIEMLDRH HTHFCPLEGESWQDFLRNNAKSFRCALLSHRDGAKVHLGTRPTEKQYETLENQLAFLCQQGFS LENALYALSAVGHFTLGCVLEDQEHQVAKEERETPTTDSMPPLLRQAIELFDHQGAEPAFLFGL ELIICGLEKQLKCESGGPADALDDFDLDMLPADALDDFDLDMLPADALDDFDLDMLPG (SEQ ID NO: 112); and has a length of from about 245 amino acids to 252 amino acids (e.g., 248, 249, 250, 251, or 252 amino acids).

In some embodiments, the intracellular domain comprises a transcriptional activator. In some cases, the transcriptional activator is GAL4-VP16. In some cases, the transcriptional activator is VP64 Zip (+). In some cases the transcriptional activator is an engineered protein, such as a zinc finger or TALE based DNA binding domain fused to an effector domain such as VP64. A variety of other transcriptional transactivators known in the art are suitable for use.

In some cases, the intracellular domain comprises an amino acid sequence having at least 75%, at least 80%, at least 85%, at least 90%, at least 95%, at least 98%, at least 99%, or 100%, amino acid sequence identity to the following GAL4-VP64 sequence:
MKLLSSIEQACDICRLKKLKCSKEKPKCAKCLKNNWECRYSPKTKRSPLTRAHLTEVESRLERL EQLFLLIFPREDLDMILKMDSLQDIKALLTGLFVQDNVNKDAVTDRLASVETDMPLTLRQHRIS ATSSSEESSNKGQRQLTVSAAAGGSGGSGGSDALDDFDLDMLGSDALDDFDLDMLGSDALDDF DLDMLGSDALDDFDLDMLGS (SEQ ID NO:113); and has a length of from 208 to 214 amino acids (e.g., 208, 209, 210, 211, 212, 213, or 214 amino acids).

In some cases, the intracellular domain comprises an amino acid sequence having at least 75%, at least 80%, at least 85%, at least 90%, at least 95%, at least 98%, at least 99%, or 100%, amino acid sequence identity to the following VP64 Zip (+) transcriptional activator sequence:
PKKKRKVDALDDFDLDMLGSDALDDFDLDMLGSDALDDFDLDMLGSDALDDFDLDMLGSGG SGGSGGSLEIEAAFLERENTALETRVAELRQRVQRLRNRVSQYRTRYGPLGGGK (SEQ ID NO: 114); and has a length of from 105 to 115 amino acids (e.g., 105, 106, 107, 108, 109, 110, 111, 112, 113, 114 or 115 amino acids).

In some instances, the intracellular domain of a proteolytically cleavable chimeric polypeptide of the instant disclosure, upon activation of the chimeric polypeptide, induces expression of a POI. A POI may be essentially any polypeptide and may include but is not limited to polypeptides of research interest (e.g., reporter polypeptides, mutated polypeptides, novel synthetic polypeptides, etc.), polypeptides of therapeutic interest (e.g., naturally occurring therapeutic proteins, recombinant therapeutic polypeptides, etc.), polypeptides of industrial interest (e.g., polypeptides used in industrial applications such as e.g., manufacturing), and the like.

In some instances, a POI may be a therapeutic polypeptide including but not limited to a therapeutic polypeptide for treating a neoplasia such as e.g., a tumor, a cancer, etc. In some instances, a therapeutic POI for treating a neoplasia may be a POI used in immunotherapy for cancer. In some instances, a therapeutic POI may be a CAR. In some instances, a therapeutic POI may be a TCR. In some instances, a therapeutic POI may be an antibody. In some instances, a therapeutic POI may be a chimeric bispecific binding member. In some instances, a therapeutic POI may be an innate-immune response inducer. In some instances, a therapeutic POI may be an immune suppression factor.

POIs of the instant disclosure include orthogonalized POIs. Orthogonalized POIs include those POIs that have been modified from their original or wild-type form such that the orthogonal POI specifically reacts with or binds a specific orthogonalized partner but does not specifically or substantially react with of bind the unmodified or wild-type partner. Any POI may be orthogonalized, including but not limited to e.g., those POIs described herein.

In some instances, a therapeutic POI may be an anti-Fc CAR. An anti-Fc CAR generally includes the extracellular domain of an Fc receptor, an intracellular signaling domain and optionally a co-stimulatory domain. Depending on the therapeutic context, an anti-Fc CAR may include an extracellular domain of any Fc receptor including e.g., a Fc-gamma receptor (e.g., FcγRI (CD64), FcγRIIA (CD32), FcγRIIB (CD32), FcγRIIIA (CD16a), FcγRIIIB (CD16b)), a Fc-alpha receptor (e.g., FcaRI (CD89)) or a Fc-epsilon receptor (e.g., FcERI, FcERII (CD23)). For example, in some instances, an anti-Fc CAR may include the extracellular domain of the CD16 Fc receptor. In some instances, an anti-Fc CAR may include the extracellular domain of the CD16 Fc receptor, a CD3-zeta intracellular signaling domain and a 4-1BB co-stimulatory domain. In some instances, an anti-Fc CAR may be an Antibody-Coupled T-cell Receptor (ACTR), e.g., as available from (Unum Therapeutics Inc.; Cambridge, MA).

In some instances, one or more domains of the anti-Fc CAR may be a mutated domain including where the domain is mutated, e.g., to modulate affinity (e.g., increase affinity or decrease affinity) for a binding partner, to modulate intracellular signaling properties (e.g., increase signaling or decrease signaling), etc.

In some instances, a chimeric polypeptide of the present disclosure may be expressed on a cell such that, upon binding the specific binding partner of the chimeric polypeptide, the intracellular domain of the chimeric polypeptide induces transcription of an anti-Fc CAR from a nucleic acid sequence within the cell. In some instances, an antibody that binds the anti-Fc CAR and a tumor antigen may be administered to a subject also administered such a cell. In some instances, a chimeric polypeptide of the present disclosure may be expressed on a cell such that, upon binding the specific binding partner of the chimeric polypeptide, the intracellular domain of the chimeric polypeptide induces transcription of an anti-Fc CAR and an antibody that binds the anti-Fc CAR and a tumor antigen from one or more nucleic acid sequences within the cell.

In some instances, a therapeutic POI may be a chimeric bispecific binding member. As used herein, by “chimeric bispecific binding member” is meant a chimeric polypeptide having dual specificity to two different binding partners (e.g., two different antigens). Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann. MAbs. (2012) 4(2): 182-197; Stamova et al. Antibodies 2012, 1(2), 172-198; Farhadfar et al. Leuk Res. (2016) 49:13-21; Benjamin et al. Ther Adv Hematol. (2016) 7(3): 142-56; Kiefer et al. Immunol Rev. (2016) 270(1): 178-92; Fan et al. J Hematol Oncol. (2015) 8:130; May et al. Am J Health Syst Pharm. (2016) 73(1):e6-e13; the disclosures of which are incorporated herein by reference in their entirety.

In some instances, a chimeric bispecific binding member may be a bispecific antibody. In some instances, a bispecific antibody that may be expressed in response to activation of a chimeric polypeptide of the present disclosure may be a bispecific antibody targeting at least one cancer antigen (including e.g., two cancer antigens) including but not limited to e.g., at least one (including e.g., two) cancer antigens described herein. In some instances, a bispecific antibody that may be expressed in response to activation of a chimeric polypeptide of the present disclosure may be a bispecific antibody targeting at one cancer antigen and one immune cell antigen including but not limited to e.g., a cancer antigen described herein and an immune antigen described herein.

In some instances, a bispecific antibody that may be expressed in response to activation of a chimeric polypeptide of the present disclosure may be e.g., bsAb MDX-210 (targeting Her2 and CD64), MDX-H210 (targeting Her2 and CD64), MDX-447 (targeting EGFR and CD64), HRS-3/A9 (a bispecific F(ab′)₂ antibody targeting the CD30 antigen and receptor FcγRIII (CD16)), an anti-CD3×anti-EpCAM TriomAb/bsAb, Catumaxomab, Ertumaxomab, Bi20 (Lymphomun or fBTA05), an anti-CD19×CD3 diabody, an anti-CD19×CD16 diabody, an anti-EGFR×CD3 diabody, an anti-PSMA×CD3 diabody, a diabody targeting rM28 and NG2, an anti-CD28×CD20 bispecific tandem scFv, or the like.

In some instances, a chimeric bispecific binding member may be a bispecific T cell engager (BITE). A BiTE is generally made by fusing a specific binding member (e.g., a scFv) that binds an immune cell antigen to a specific binding member (e.g., a scFv) that binds a cancer antigen (e.g., a tumor associated antigen, a tumor specific antigen, etc.). For example, an exemplary BiTE includes an anti-CD3 scFv fused to an anti-tumor associated antigen (e.g., EpCAM, CD19, etc.) scFv via a short peptide linker (e.g., a five amino acid linker, e.g., GGGGS (SEQ ID NO:115)).

In some instances, a BiTE that may be expressed in response to activation of a chimeric polypeptide of the present disclosure may be a BiTE targeting at least one cancer antigen including but not limited to e.g., a cancer antigen described herein. In some instances, a BiTE that may be expressed in response to activation of a chimeric polypeptide of the present disclosure may be a BiTE targeting one cancer antigen and one immune cell antigen including but not limited to e.g., a cancer antigen described herein and an immune antigen described herein.

In some instances, a chimeric polypeptide of the present disclosure may be expressed on a cell such that, upon binding the specific binding partner of the chimeric polypeptide, the intracellular domain of the chimeric polypeptide induces transcription of a BiTE from a nucleic acid sequence within the cell. In some instances, a chimeric polypeptide of the present disclosure may be expressed on a cell such that, upon binding a peptide-MHC specific binding partner, the intracellular domain of the chimeric polypeptide induces transcription of a BiTE. In some instances, a BiTE suitable for use as herein described includes e.g., an anti-CD3×anti-CD19 BiTE (e.g., Blinatumomab), an anti-EpCAM×anti-CD3 BiTE (e.g., MT110), an anti-CEA×anti-CD3 BiTE (e.g., MT111/MEDI-565), an anti-CD33×anti-CD3 BiTE, an anti-HER2 BiTE, an anti-EGFR BiTE, an anti-IgE BiTE, and the like.

In some instances, a chimeric bispecific binding member may be a Micabody or mutant thereof. A Micabody generally includes an antigen-specific binding portion linked to at least one domain that specifically binds a NKG2D receptor. In some instances, a Micabody or mutant thereof includes engineered MICA α1-α2 domains that specifically bind to NKG2D receptors.

In some instances, a Micabody or mutant thereof that may be expressed in response to activation of a chimeric polypeptide of the present disclosure may be a Micabody or mutant thereof targeting at least one cancer antigen including but not limited to e.g., a cancer antigen described herein. In some instances, a Micabody or mutant thereof that may be expressed in response to activation of a chimeric polypeptide of the present disclosure may be a Micabody or mutant thereof targeting HER2 (e.g., an anti-HER2 Micabody or mutants thereof). Non-limiting examples of Micabodies and related components and operating principles are described in e.g., Cho et al., Cancer Res. (2010) 70(24): 10121-30; Bauer et al. Science. (1999) 285(5428): 727-9; Morvan et al. Nat Rev Cancer. (2016) 16(1): 7-19; the disclosures of which are incorporated herein by reference in their entirety.

In some instances, a chimeric polypeptide of the present disclosure may be expressed on a cell such that, upon binding the specific binding partner of the chimeric polypeptide, the intracellular domain of the chimeric polypeptide induces transcription of a Micabody or mutant thereof from a nucleic acid sequence within the cell. In some instances, a chimeric polypeptide of the present disclosure may be expressed on a cell such that, upon binding a peptide-MHC specific binding partner, the intracellular domain of the chimeric polypeptide induces transcription of a Micabody or mutant thereof. Micabodies and mutants thereof include those developed by AvidBiotics (South San Francisco, CA) and described online at (avidbiotics(dot)com).

In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have to binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. J Am Chem Soc. (2015) 137(8): 2832-5; Ma et al. Proc Natl Acad Sci USA. (2016) 113(4): E450-8 and Cao et al. Angew Chem Int Ed Engl. (2016) 55(26): 7520-4; the disclosures of which are incorporated herein by reference in their entirety.

In some cases, a therapeutic POI that is induced by a chimeric polypeptide of the instant disclosure is an antibody. Suitable antibodies include, e.g., Natalizumab (Tysabri; Biogen Idec/Elan) targeting α4 subunit of α4β1 and α4β7 integrins (as used in the treatment of MS and Crohn's disease); Vedolizumab (MLN2; Millennium Pharmaceuticals/Takeda) targeting α4β7 integrin (as used in the treatment of UC and Crohn's disease); Belimumab (Benlysta; Human Genome Sciences/GlaxoSmithKline) targeting BAFF (as used in the treatment of SLE); Atacicept (TACI-Ig; Merck/Serono) targeting BAFF and APRIL (as used in the treatment of SLE); Alefacept (Amevive; Astellas) targeting CD2 (as used in the treatment of Plaque psoriasis, GVHD); Otelixizumab (TRX4; Tolerx/GlaxoSmithKline) targeting CD3 (as used in the treatment of T1D); Teplizumab (MGA031; MacroGenics/Eli Lilly) targeting CD3 (as used in the treatment of T1D); Rituximab (Rituxan/Mabthera; Genentech/Roche/Biogen Idec) targeting CD20 (as used in the treatment of Non-Hodgkin's lymphoma, RA (in patients with inadequate responses to TNF blockade) and CLL); Ofatumumab (Arzerra; Genmab/GlaxoSmithKline) targeting CD20 (as used in the treatment of CLL, RA); Ocrelizumab (2H7; Genentech/Roche/Biogen Idec) targeting CD20 (as used in the treatment of R^{A} and SLE); Epratuzumab (hLL2; Immunomedics/UCB) targeting CD22 (as used in the treatment of SLE and non-Hodgkin's lymphoma); Alemtuzumab (Campath/MabCampath; Genzyme/Bayer) targeting CD52 (as used in the treatment of CLL, MS); Abatacept (Orencia; Bristol-Myers Squibb) targeting CD80 and CD86 (as used in the treatment of R^{A} and JIA, UC and Crohn's disease, SLE); Eculizumab (Soliris; Alexion pharmaceuticals) targeting C5 complement protein (as used in the treatment of Paroxysmal nocturnal haemoglobinuria); Omalizumab (Xolair; Genentech/Roche/Novartis) targeting IgE (as used in the treatment of Moderate to severe persistent allergic asthma); Canakinumab (Ilaris; Novartis) targeting IL-1B (as used in the treatment of Cryopyrin-associated periodic syndromes, Systemic JIA, neonatal-onset multisystem inflammatory disease and acute gout); Mepolizumab (Bosatria; GlaxoSmithKline) targeting IL-5 (as used in the treatment of Hyper-eosinophilic syndrome); Reslizumab (SCH55700; Ception Therapeutics) targeting IL-5 (as used in the treatment of Eosinophilic oesophagitis); Tocilizumab (Actemra/RoActemra; Chugai/Roche) targeting IL-6R (as used in the treatment of RA, JIA); Ustekinumab (Stelara; Centocor) targeting IL-12 and IL-23 (as used in the treatment of Plaque psoriasis, Psoriatic arthritis, Crohn's disease); Briakinumab (ABT-874; Abbott) targeting IL-12 and IL-23 (as used in the treatment of Psoriasis and plaque psoriasis); Etanercept (Enbrel; Amgen/Pfizer) targeting TNF (as used in the treatment of RA, JIA, psoriatic arthritis, AS and plaque psoriasis); Infliximab (Remicade; Centocor/Merck) targeting TNF (as used in the treatment of Crohn's disease, RA, psoriatic arthritis, UC, AS and plaque psoriasis); Adalimumab (Humira/Trudexa; Abbott) targeting TNF (as used in the treatment of RA, JIA, psoriatic arthritis, Crohn's disease, AS and plaque psoriasis); Certolizumab pegol (Cimzia; UCB) targeting TNF (as used in the treatment of Crohn's disease and RA); Golimumab (Simponi; Centocor) targeting TNF (as used in the treatment of RA, psoriatic arthritis and AS); and the like.

In some cases, the antibody whose production is induced is a therapeutic antibody for the treatment of cancer. Such antibodies include, e.g., Ipilimumab targeting CTLA-4 (as used in the treatment of Melanoma, Prostate Cancer, RCC); Tremelimumab targeting CTLA-4 (as used in the treatment of CRC, Gastric, Melanoma, NSCLC); Nivolumab targeting PD-1 (as used in the treatment of Melanoma, NSCLC, RCC); MK-3475 targeting PD-1 (as used in the treatment of Melanoma); Pidilizumab targeting PD-1 (as used in the treatment of Hematologic Malignancies); BMS-936559 targeting PD-L1 (as used in the treatment of Melanoma, NSCLC, Ovarian, RCC); MEDI4736 targeting PD-L1; MPDL33280A targeting PD-L1 (as used in the treatment of Melanoma); Rituximab targeting CD20 (as used in the treatment of Non-Hodgkin's lymphoma); Ibritumomab tiuxetan and tositumomab (as used in the treatment of Lymphoma); Brentuximab vedotin targeting CD30 (as used in the treatment of Hodgkin's lymphoma); Gemtuzumab ozogamicin targeting CD33 (as used in the treatment of Acute myelogenous leukaemia); Alemtuzumab targeting CD52 (as used in the treatment of Chronic lymphocytic leukaemia); IGN101 and adecatumumab targeting EpCAM (as used in the treatment of Epithelial tumors (breast, colon and lung)); Labetuzumab targeting CEA (as used in the treatment of Breast, colon and lung tumors); huA33 targeting gpA33 (as used in the treatment of Colorectal carcinoma); Pemtumomab and oregovomab targeting Mucins (as used in the treatment of Breast, colon, lung and ovarian tumors); CC49 (minretumomab) targeting TAG-72 (as used in the treatment of Breast, colon and lung tumors); cG250 targeting CAIX (as used in the treatment of Renal cell carcinoma); J591 targeting PSMA (as used in the treatment of Prostate carcinoma); MOv18 and MORAb-003 (farletuzumab) targeting Folate-binding protein (as used in the treatment of Ovarian tumors); 3F8, ch14.18 and KW-2871 targeting Gangliosides (such as GD2, GD3 and GM2) (as used in the treatment of Neuroectodermal tumors and some epithelial tumors); hu3S193 and IgN311 targeting Le y (as used in the treatment of Breast, colon, lung and prostate tumors); Bevacizumab targeting VEGF (as used in the treatment of Tumor vasculature); IM-2C6 and CDP791 targeting VEGFR (as used in the treatment of Epithelium-derived solid tumors); Etaracizumab targeting Integrin_V_3 (as used in the treatment of Tumor vasculature); Volociximab targeting Integrin_5_1 (as used in the treatment of Tumor vasculature); Cetuximab, panitumumab, nimotuzumab and 806 targeting EGFR (as used in the treatment of Glioma, lung, breast, colon, and head and neck tumors); Trastuzumab and pertuzumab targeting ERBB2 (as used in the treatment of Breast, colon, lung, ovarian and prostate tumors); MM-121 targeting ERBB3 (as used in the treatment of Breast, colon, lung, ovarian and prostate, tumors); AMG 102, METMAB and SCH 900105 targeting MET (as used in the treatment of Breast, ovary and lung tumors); AVE1642, IMC-A12, MK-0646, R1507 and CP 751871 targeting IGFIR (as used in the treatment of Glioma, lung, breast, head and neck, prostate and thyroid cancer); KB004 and IIIA4 targeting EPHA3 (as used in the treatment of Lung, kidney and colon tumors, melanoma, glioma and haematological malignancies); Mapatumumab (HGS-ETR1) targeting TRAILR1 (as used in the treatment of Colon, lung and pancreas tumors and haematological malignancies); HGS-ETR2 and CS-1008 targeting TRAILR2; Denosumab targeting RANKL (as used in the treatment of Prostate cancer and bone metastases); Sibrotuzumab and F19 targeting FAP (as used in the treatment of Colon, breast, lung, pancreas, and head and neck tumors); 81C6 targeting Tenascin (as used in the treatment of Glioma, breast and prostate tumors); Blinatumomab (Blincyto; Amgen) targeting CD3 (as used in the treatment of ALL); pembrolizumab targeting PD-1 as used in cancer immunotherapy; 9E10 antibody targeting c-Myc; and the like.

In some cases, useful antibodies, the expression of which can be induced by a chimeric polypeptide of the instant disclosure, include but are not limited to 8H9, Abagovomab, Abciximab, Abituzumab, Abrilumab, Actoxumab, Aducanumab, Afelimomab, Afutuzumab, Alacizumab pegol, ALD518, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atezolizumab, Atinumab, Atlizumab/tocilizumab, Atorolimumab, Bapineuzumab, Basiliximab, Bavituximab, Bectumomab, Begelomab, Benralizumab, Bertilimumab, Besilesomab, Bevacizumab/Ranibizumab, Bezlotoxumab, Biciromab, Bimagrumab, Bimekizumab, Bivatuzumab mertansine, Blosozumab, Bococizumab, Brentuximabvedotin, Brodalumab, Brolucizumab, Brontictuzumab, Cantuzumab mertansine, Cantuzumab ravtansine, Caplacizumab, Capromab pendetide, Carlumab, Catumaxomab, cBR96-doxorubicin immunoconjugate, Cedelizumab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CR6261, Crenezumab, Dacetuzumab, Daclizumab, Dalotuzumab, Dapirolizumab pegol, Daratumumab, Dectrekumab, Demcizumab, Denintuzumab mafodotin, Derlotuximab biotin, Detumomab, Dinutuximab, Diridavumab, Dorlimomab aritox, Drozitumab, Duligotumab, Dupilumab, Durvalumab, Dusigitumab, Ecromeximab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Erlizumab, Ertumaxomab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gevokizumab, Girentuximab, Glembatumumab vedotin, Gomiliximab, Guselkumab, Ibalizumab, Ibalizumab, Icrucumab, Idarucizumab, Igovomab, IMAB362, Imalumab, Imciromab, Imgatuzumab, Inclacumab, Indatuximab ravtansine, Indusatumab vedotin, Inolimomab, Inotuzumab ozogamicin, Intetumumab, Iratumumab, Isatuximab, Itolizumab, Ixekizumab, Keliximab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Margetuximab, Maslimomab, Matuzumab, Mavrilimumab, Metelimumab, Milatuzumab, Minretumomab, Mirvetuximab soravtansine, Mitumomab, Mogamulizumab, Morolimumab, Morolimumab immune, Motavizumab, Moxetumomab pasudotox, Muromonab-CD3, Nacolomab tafenatox, Namilumab, Naptumomab estafenatox, Narnatumab, Nebacumab, Necitumumab, Nemolizumab, Nerelimomab, Nesvacumab, Nofetumomab merpentan, Obiltoxaximab, Obinutuzumab, Ocaratuzumab, Odulimomab, Olaratumab, Olokizumab, Onartuzumab, Ontuxizumab, Opicinumab, Oportuzumab monatox, Orticumab, Otlertuzumab, Oxelumab, Ozanezumab, Ozoralizumab, Pagibaximab, Palivizumab, Pankomab, Panobacumab, Parsatuzumab, Pascolizumab, Pasotuxizumab, Pateclizumab, Patritumab, Perakizumab, Pexelizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ralpancizumab, Ramucirumab, Ranibizumab, Raxibacumab, Refanezumab, Regavirumab, Rilotumumab, Rinucumab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teprotumumab, Tesidolumab, Tetulomab, TGN1412, Ticilimumab/tremelimumab, Tigatuzumab, Tildrakizumab, TNX-650, Toralizumab, Tosatoxumab, Tovetumab, Tralokinumab, TRBS07, Tregalizumab, Trevogrumab, Tucotuzumab celmoleukin, Tuvirumab, Ublituximab, Ulocuplumab, Urelumab, Urtoxazumab, Vandortuzumab vedotin, Vantictumab, Vanucizumab, Vapaliximab, Varlilumab, Vatelizumab, Veltuzumab, Vepalimomab, Vesencumab, Visilizumab, Vorsetuzumab mafodotin, Votumumab, Zalutumumab, Zanolimumab, Zatuximab, Ziralimumab, Zolimomab aritox, and the like.

In some instances, a proteolytically cleavable chimeric polypeptide of the instant disclosure may induce the expression of a T-cell receptor (TCR) in a cell. Any TCR can be induced by a chimeric polypeptide using a method of the present disclosure including e.g., TCRs that are specific for any of a variety of epitopes, including, e.g., an epitope expressed on the surface of a cancer cell, a peptide-MHC complex on the surface of cancer cell, and the like. A TCR generally includes an alpha chain and a beta chain; and recognizes antigen when presented by a major histocompatibility complex. In some cases, the TCR is an engineered TCR.

Any engineered TCR having immune cell activation function can be induced using a method of the present disclosure. Such TCRs include, e.g., antigen-specific TCRs, Monoclonal TCRs (MTCRs), Single chain MTCRs, High Affinity CDR2 Mutant TCRs, CD1-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1): 145-64; Schmitt et al. Blood. 2013; 122(3): 348-56; Chapuls et al. Sci Transl Med. 2013; 5(174): 174ra27; Thaxton et al. Hum Vaccin Immunother. 2014; 10(11): 3313-21 (PMID: 25483644); Gschweng et al. Immunol Rev. 2014; 257(1): 237-49 (PMID: 24329801); Hinrichs et al. Immunol Rev. 2014; 257(1): 56-71 (PMID: 24329789); Zoete et al. Front Immunol. 2013; 4:268 (PMID: 24062738); Marr et al. Clin Exp Immunol. 2012; 167(2): 216-25 (PMID: 22235997); Zhang et al. Adv Drug Deliv Rev. 2012; 64(8): 756-62 (PMID: 22178904); Chhabra et al. Scientific World Journal. 2011; 11:121-9 (PMID: 21218269); Boulter et al. Clin Exp Immunol. 2005; 142(3): 454-60 (PMID: 16297157); Sami et al. Protein Eng Des Sel. 2007; 20(8): 397-403; Boulter et al. Protein Eng. 2003; 16(9): 707-11; Ashfield et al. IDrugs. 2006; 9(8): 554-9; Li et al. Nat Biotechnol. 2005; 23(3): 349-54; Dunn et al. Protein Sci. 2006; 15(4): 710-21; Liddy et al. Mol Biotechnol. 2010; 45(2); Liddy et al. Nat Med. 2012; 18(6): 980-7; Oates, et al. Oncoimmunology. 2013; 2(2):e22891; McCormack, et al. Cancer Immunol Immunother. 2013 April; 62(4): 773-85; Bossi et al. Cancer Immunol Immunother. 2014; 63(5): 437-48 and Oates, et al. Mol Immunol. 2015 October; 67(2 Pt A): 67-74; the disclosures of which are incorporated herein by reference in their entirety.

In some instances, a chimeric polypeptide of the instant disclosure induces expression of an engineered TCR targeting a cancer antigen, including e.g., an intracellular cancer antigen. In some instances, an engineered TCR induced to be expressed by a chimeric polypeptide of the instant disclosure is an engineered TCR targeting an antigen target listed in Table 2 below.

TABLE 2
Engineered TCR Targets

| Target | HLA | References |
|---|---|---|
| NY-ESO-1 | HLA-A2 | J Immunol. (2008) 180(9): 6116-31 |
| MART-1 | HLA-A2 | J Immunol. (2008) 180(9): 6116-31; |
| | | Blood. (2009) 114(3): 535-46 |
| MAGE-A3 | HLA-A2 | J Immunother. (2013) 36(2): 133-51 |
| MAGE-A3 | HLA-A1 | Blood. (2013) 122(6): 863-71 |
| CEA | HLA-A2 | Mol Ther. (2011) 19(3): 620-626 |
| gp100 | HLA-A2 | Blood. (2009) 114(3): 535-46 |
| WT1 | HLA-A2 | Blood. (2011) 118(6): 1495-503 |
| HBV | HLA-A2 | J Hepatol. (2011) 55(1): 103-10 |
| gag (WT and/or α/6) | HLA-A2 | Nat Med. (2008) 14(12): 1390-5 |
| P53 | HLA-A2 | Hum Gene Ther. (2008) 19(11): 1219-32 |
| TRAIL bound to DR4 | N/A | J Immunol. (2008) 181(6): 3769-76 |
| HPV-16 (E6 and/or E7) | HLA-A2 | Clin Cancer Res. (2015) 21(19): 4431-9 |
| Survivin | HLA-A2 | J Clin Invest. (2015) 125(1): 157-68 |
| KRAS mutants | HLA-A11 | Cancer Immunol Res. (2016) 4(3): 204-14 |
| SSX2 | HLA-A2 | PLoS One. (2014) 9(3): e93321 |
| MAGE-A10 | HLA-A2 | J ImmunoTherapy Cancer. (2015) 3(Suppl2): P14 |
| MAGE-A4 | HLA-A24 | Clin Cancer Res. (2015) 21(10): 2268-77 |
| AFP | HLA-A2 | J ImmunoTherapy Cancer. (2013) 1(Suppl1): P10 |

In some instances, an expressed TCR targeting a particular antigen may be described as an anti-[antigen] TCR. Accordingly, in some instances, exemplary TCRs that may be induced to be expressed by a chimeric polypeptide of the instant disclosure include but are not limited to e.g., an anti-NY-ESO-1 TCR; an anti-MART-1 TCR; an anti-MAGE-A3 TCR; an anti-MAGE-A3 TCR; an anti-CEA TCR; an anti-gp100 TCR; an anti-WT1 TCR; an anti-HBV TCR; an anti-gag (WT and/or a/6) TCR; an anti-P53 TCR; an anti-TRAIL bound to DR4 TCR; an anti-HPV-16 (E6 and/or E7) TCR; an anti-Survivin TCR; an anti-KRAS mutants TCR; an anti-SSX2 TCR; an anti-MAGE-A10 TCR; an anti-MAGE-A4 TCR; an anti-AFP TCR; and the like.

In some instances, the TCR is an anti-NY-ESO1 TCR (e.g., an anti-HLA-A2/NY-ESO1 scTv). In some instances, the anti-NY-ESO1 TCR has the following sequence:

(SEQ ID NO: 116)
METLLGLLILWLQLQWVSSKQEVTQIPAALSVPEGENLVLNCSFTDSAIY
NLQWFRQDPGKGLTSLLLIQSSQREQTSGRLNASLDKSSGRSTLYIAASQ
PGDSATYLCAVRPLLDGTYIPTFGRGTSLIVHPGSADDAKKDAAKKDGKS
MSIGLLCCAALSLLWAGPVNAGVTQTPKFQVLKTGQSMTLQCAQDMNHEY
MSWYRQDPGMGLRLIHYSVGAGTTDRGEVPNGYNVSRSTIEDFPLRLLSA
APSQTSVYFCASSYVGDTGELFFGEGSRLTVL.

Useful TCRs include those having wild-type affinity for their respective antigen as well as those having enhanced affinity for their respective antigen. TCRs having enhanced affinity for their respective antigen may be referred to as “affinity enhanced” or “enhanced affinity” TCRs. The affinity of a TCR may enhanced by any convenient means, including but not limited to binding-site engineering (i.e., rational design), screening (e.g., TCR display), or the like. Non-limiting examples of affinity enhanced TCRs and methods of generating enhanced affinity TCRs include but are not limited to e.g., those described in PCT Pub. Nos. 20150118208, 2013256159, 20160083449; 20140349855, 20100113300, 20140371085, 20060127377, 20080292549, 20160280756, 20140065111, 20130058908, 20110038842, 20110014169, 2003276403 and the like; the disclosures of which are incorporated herein by reference in their entirety.

Useful TCRs may, in some instances, also include modified TCR chains that include one or more cysteine modifications. Such cysteine modifications may be paired between two modified TCR chains. When paired between two TCR chains, corresponding modifications may result in a recombinant disulfide bond between the paired chains.

In some embodiments, a TCR may include a first cysteine modification in an alpha chain and a second cysteine modification in a beta chain where the first and second cysteine modifications, when both chains are present in a cell, form a recombinant disulfide bond between the alpha chain and the beta chain. Such cysteine modifications that form a recombinant disulfide bond may be referred to as “corresponding cysteine modifications”.

In some instances, a modified TCR alpha chain may include a substitution of a residue to a cysteine resulting in a cysteine modification sufficient to produce a recombinant disulfide bond. Any appropriate residue of a TCR alpha chain having a corresponding residue in a TCR beta chain that, when mutated to a cysteine results in a recombinant disulfide bond, may be employed in generating a cysteine modified alpha chain. In some instances, the substituted residue is a residue present in the TCR alpha constant region. In some instances, the substitution is a tyrosine to cysteine substitution. In some instances, the substitution is a T48C substitution, or corresponding mutation, such as the T48C substitution present in the following human TCR alpha chain constant region sequence:
PNIQNPDPAVYQLRDSKSSDKSVCLFTDFDSQTNVSQSKDSDVYITDKCVLDMRSMDFKSNSAV AWSNKSDFACANAFNNSIIPEDTFFPSPESSCDVKLVEKSFETDTNLNFQNLSVIGFRILLLKVAG FNLLMTLRLWSS (SEQ ID NO:131). In some instances, the substitution is a T84C substitution, or corresponding mutation, such as the T84C substitution present in the following mouse TCR alpha chain constant region sequence:

(SEQ ID NO: 132)
PYIQNPEPAVYQLKDPRSQDSTLCLFTDFDSQINVPKTMESGTFITDKTV
LDMKAMDSKSNGAIAWSNQTSFTCQDIFKETNACYPSSDVPCDATLTEKS
FETDMNLNFQNLSVMGLRILLLKVAGFNLLMTLRLWSS.

In some instances, a subject TCR alpha chain or corresponding domain thereof (e.g., an alpha variable domain, an alpha constant domain, an alpha transmembrane domain, an alpha connecting peptide domain, and the like), may have at least about 75%, at least about 80%, at least about 85%, at least about 90%, at least about 95%, at least about 98%, or 100% amino acid sequence identity to a cysteine modified alpha chain, such as e.g., the T48C or T84C containing TCR alpha sequences provided above.

In some instances, a modified TCR beta chain may include a substitution of a residue to a cysteine resulting in a cysteine modification sufficient to produce a recombinant disulfide bond. Any appropriate residue of a TCR beta chain having a corresponding residue in a TCR beta chain that, when mutated to a cysteine results in a recombinant disulfide bond, may be employed in generating a cysteine modified beta chain. In some instances, the substituted residue is a residue present in the TCR beta constant region. In some instances, the substitution is a serine to cysteine substitution. In some instances, the substitution is a S58C substitution, or corresponding mutation, such as the S58C substitution present in the following human TCR beta chain constant region sequence:

EDLNKVFPPEVAVFEPSEAEISHTQKATLVCLATGFFPDHVELSWWVNGKEVHSGVCTDPQPLK EQPALNDSRYCLSSRLRVSATFWQNPRNHFRCQVQFYGLSENDEWTQDRAKPVTQIVSAEAWG RADCGFTSVSYQQGVLSATILYEILLGKATLYAVLVSALVLMAMVKRKDF (SEQ ID NO:133). In some instances, the substitution is a S79C substitution, or corresponding mutation, such as the S79C substitution present in the following mouse TCR beta chain constant region sequence:

(SEQ ID NO: 134)
EDLRNVTPPKVSLFEPSKAEIANKQKATLVCLARGFFPDHVELSWWVNGK
EVHSGVSTDPQAYKESNYSYCLSSRLRVCATFWHNPRNHFRCQVQFHGLS
EEDKWPEGSPKPVTQNISAEAWGRADCGITSASYQQGVLSATILYEILLG
KATLYAVLVSTLVVMAMVKRKNS.

In some instances, a subject TCR beta chain or corresponding domain thereof (e.g., a beta variable domain, a beta constant domain, a beta transmembrane domain, a beta connecting peptide domain, and the like), may have at least about 75%, at least about 80%, at least about 85%, at least about 90%, at least about 95%, at least about 98%, or 100% amino acid sequence identity to a cysteine modified beta chain, such as e.g., the S58C or S79C containing TCR beta sequences provided above.

In some instances, a therapeutic POI may be an innate-immune response inducer. As used herein, by “innate-immune response inducer” is meant any protein that when expressed within a mammal induces an innate immune response. Innate immune inducers include but are not limited to e.g., proteins or fragments thereof derived from bacteria, proteins or fragments thereof derived from virus, proteins or fragments thereof derived from fungus, proteins or fragments thereof derived from a mammalian parasite, including e.g., human parasites. Any protein that induces an innate immune response when expressed by a mammalian cell may find use as an innate-immune inducer of the instant disclosure. In some instances, an innate immune response inducer may be a flagellin protein.

In some instances, a chimeric polypeptide of the present disclosure may be expressed on a cell such that, upon binding the specific binding partner of the chimeric polypeptide, the intracellular domain of the chimeric polypeptide induces transcription of an innate-immune response inducer from a nucleic acid sequence within the cell. In some instances, a chimeric polypeptide of the present disclosure may be expressed on a cell such that, upon binding a peptide-MHC specific binding partner, the intracellular domain of the chimeric polypeptide induces transcription of an innate-immune response inducer.

In some instances, a therapeutic POI may be an immune suppression factor. As used herein, by “immune suppression factor” is meant any protein that when expressed within a mammal suppresses an immune response. Immune suppression factors include but are not limited to e.g., immunosuppressive cytokines (e.g., IL-10), immunosuppressive cell-to-cell signaling ligands (e.g., PD-L1), immunosuppressive secreted proteins (e.g., TGF-beta), immunosuppressive antibodies (e.g., anti-CD3 antibodies (e.g., Orthoclone OKT3 (also known as Muromonab-CD3), etc.), anti-CD25 antibodies, (e.g., Basiliximab, Daclizumab, etc.) anti-CD52 antibodies (e.g., Campath-1H (also known as alemtuzumab), etc.), and the like. Any protein that suppresses an immune response when expressed by a mammalian cell may find use as an immune suppression factor of the instant disclosure. In some instances, an immune suppression factor may be IL-10. In some instances, an immune suppression factor may be PD-L1. In some instances, an immune suppression factor may be TGF-beta. In some instances, an immune suppression factor may be an immunosuppressive antibody (e.g., (e.g., an anti-CD3 antibody (e.g., Orthoclone OKT3 (also known as Muromonab-CD3), etc.), an anti-CD25 antibody, (e.g., Basiliximab, Daclizumab, etc.) anti-CD52 antibody (e.g., Campath-1H (also known as alemtuzumab), etc.).

In some instances, a chimeric polypeptide may drive expression of two or more immune suppression factors including e.g., an immunosuppressive cytokine and an immunosuppressive cell-to-cell signaling ligand, two or more immunosuppressive cytokines, two or more immunosuppressive cell-to-cell signaling ligands, etc. In some instances, a chimeric polypeptide may drive expression of both IL-10 and PD-L1. In some instances, a chimeric polypeptide may drive expression of three or more immune suppression factors.

In some instances, a chimeric polypeptide of the present disclosure may be expressed on a cell such that, upon binding the specific binding partner of the chimeric polypeptide, the intracellular domain of the chimeric polypeptide induces transcription of an immune suppression factor from a nucleic acid sequence within the cell. In some instances, a chimeric polypeptide of the present disclosure may be expressed on a cell such that, upon binding a peptide-MHC specific binding partner, the intracellular domain of the chimeric polypeptide induces transcription of an immune suppression factor.

In some instances, a therapeutic POI may be chemokine. An expressed chemokine may affect one or more cellular behaviors including but not limited to cell migration. In some instances, the intracellular domain of a chimeric receptor polypeptide of the present disclosure may induce expression of a chemokine. Examples of suitable chemokines include, e.g., MIP-1, MIP-1β, MCP-1, RANTES, IP10, and the like. Additional examples of suitable chemokines include, but are not limited to, chemokine (C—C motif) ligand-2 (CCL2; also referred to as monocyte chemotactic protein-1 or MCP1); chemokine (C—C motif) ligand-3 (CCL3; also known as macrophage inflammatory protein-1A or MIP1A); chemokine (C—C motif) ligand-5 (CCL5; also known as RANTES); chemokine (C—C motif) ligand-17 (CCL17; also known as thymus and activation regulated chemokine or TARC); chemokine (C—C motif) ligand-19 (CCL19; also known as EBI1 ligand chemokine or ELC); chemokine (C—C motif) ligand-21 (CCL21; also known as 6Ckine); C—C chemokine receptor type 7 (CCR7); chemokine (C—X—C motif) ligand 9 (CXCL9; also known as monokine induced by gamma interferon or MIG); chemokine (C—X—C motif) ligand 10 (CXCL10; also known as interferon gamma-induced protein 10 or IP-10); chemokine (C—X—C motif) ligand 11 (CXCL11; also called interferon-inducible T-cell alpha chemoattractant or I-TAC); chemokine (C—X—C motif) ligand 16 (CXCL16; chemokine (C motif) ligand (XCL1; also known as lymphotactin); and macrophage colony-stimulating factor (MCSF).

Certain intracellular domains and components thereof that may be adapted for use in chimeric polypeptides and the methods and circuits described herein include but are not limited to e.g., those described in PCT Application No. US2016/019188 (Pub. No. WO 2016/138034), the disclosure of which is incorporated herein by reference in its entirety.

### Additional Polypeptides

A chimeric polypeptide of the present disclosure can further include one or more additional polypeptides, where suitable additional polypeptides include, but are not limited to, a signal sequence; an epitope tag; an affinity domain; a nuclear localization signal (NLS); and a polypeptide that produces a detectable signal. One or more additional sequences may be appended to the chimeric polypeptide at essentially any location where appropriate including e.g., at the N-terminus, at the C-terminus, between two domains (e.g., between the extracellular domain and the transmembrane domain, between the extracellular domain and the Notch receptor polypeptide, between the Notch receptor polypeptide and the intracellular signaling domain, etc.). Additional sequences may function with a chimeric polypeptide independently of other domains or may be associated with and function together with any domain of the chimeric polypeptide.

Signal sequences that are suitable for use in a chimeric polypeptide of the present disclosure include any eukaryotic signal sequence, including a naturally-occurring signal sequence, a synthetic (e.g., man-made) signal sequence, etc.

Suitable epitope tags include, but are not limited to, hemagglutinin (HA; e.g., YPYDVPDYA (SEQ ID NO:117); FLAG (e.g., DYKDDDDK (SEQ ID NO:118); c-myc (e.g., EQKLISEEDL; SEQ ID NO: 119), and the like.

Affinity domains include peptide sequences that can interact with a binding partner, e.g., such as one immobilized on a solid support, useful for identification or purification. Multiple consecutive single amino acids, such as histidine, when fused to a chimeric polypeptide of the present disclosure, may be used for one-step purification of the recombinant chimeric polypeptide by high affinity binding to a resin column, such as nickel sepharose. Exemplary affinity domains include His5 (HHHHH) (SEQ ID NO: 120), HisX6 (HHHHHH) (SEQ ID NO:121), C-myc (EQKLISEEDL) (SEQ ID NO:119), Flag (DYKDDDDK) (SEQ ID NO:118), StrepTag (WSHPQFEK) (SEQ ID NO:122), hemagglutinin, e.g., HA Tag (YPYDVPDYA) (SEQ ID NO:117), GST, thioredoxin, cellulose binding domain, RYIRS (SEQ ID NO: 123), Phe-His-His-Thr (SEQ ID NO:124), chitin binding domain, S-peptide, T7 peptide, SH2 domain, C-end RNA tag, WEAAAREACCRECCARA (SEQ ID NO:125), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

Suitable nuclear localization signals (“NLS”; also referred to herein as “nuclear localization sequences”) include, e.g., PKKKRKV (SEQ ID NO:126); KRPAATKKAGQAKKKK (SEQ ID NO: 127); MVPKKKRK (SEQ ID NO:128); MAPKKKRKVGIHGVPAA (SEQ ID NO:129); and the like. An NLS can be present at the N-terminus of a chimeric polypeptide of the present disclosure; near the N-terminus of a chimeric polypeptide of the present disclosure (e.g., within 5 amino acids, within 10 amino acids, or within 20 amino acids of the N-terminus); at the C-terminus of a chimeric polypeptide of the present disclosure; near the C-terminus of a chimeric polypeptide of the present disclosure (e.g., within 5 amino acids, within 10 amino acids, or within 20 amino acids of the C-terminus); or internally within a chimeric polypeptide of the present disclosure.

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

Certain additional polypeptides and components thereof that may be adapted for use in the chimeric polypeptides and the methods and circuits described herein include but are not limited to e.g., those described in PCT Application No. US2016/019188 (Pub. No. WO 2016/138034), the disclosure of which is incorporated herein by reference in its entirety.

### Nucleic Acids

The present disclosure provides a nucleic acid comprising a nucleotide sequence encoding a proteolytically cleavable chimeric polypeptide of the present disclosure. In some cases, a nucleic acid comprising a nucleotide sequence encoding a chimeric polypeptide of the present disclosure is contained within an expression vector. Thus, the present disclosure provides a recombinant expression vector comprising a nucleic acid comprising a nucleotide sequence encoding a chimeric polypeptide of the present disclosure. In some cases, the nucleotide sequence encoding a chimeric polypeptide of the present disclosure is operably linked to a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are cell type-specific promoters. In some cases, the promoters are tissue-specific promoters.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoter and enhancer elements are known in the art. For expression in a bacterial cell, suitable promoters include, but are not limited to, lacl, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

In some instances, a transcriptional control element of a herein described nucleic acid may include a cis-acting regulatory sequence. Any suitable cis-acting regulatory sequence may find use in the herein described nucleic acids. For example, in some instances a cis-acting regulatory sequence may be or include an upstream activating sequence or upstream activation sequence (UAS). In some instances, a UAS of a herein described nucleic acid may be a Gal4 responsive UAS.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of an Ncrl (p46) promoter; see, e.g., Eckelhart et al. (2011) Blood 117:1565.

In some cases, a nucleic acid comprising a nucleotide sequence encoding a chimeric polypeptide of the present disclosure is a recombinant expression vector or is included in a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a chimeric polypeptide of the present disclosure is a recombinant lentivirus vector. In some cases, a nucleic acid comprising a nucleotide sequence encoding a chimeric polypeptide of the present disclosure is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

Nucleic acids of the instant disclosure may include nucleic acid sequence encoding a polypeptide of interest (POI). A POI may be essentially any polypeptide and may include but is not limited to polypeptides of research interest (e.g., reporter polypeptides, mutated polypeptides, novel synthetic polypeptides, etc.), polypeptides of therapeutic interest (e.g., naturally occurring therapeutic proteins, recombinant therapeutic polypeptides, etc.), polypeptides of industrial interest (e.g., polypeptides used in industrial applications such as e.g., manufacturing), and the like.

In some instances, a POI may be a transcriptional activator. In some instances, a POI may be a CAR. In some instances, a POI may be a TCR. In some instances, a POI may be an antibody. In some instances, a POI may be a chimeric bispecific binding member. In some instances, a POI may be an innate-immune response inducer. In some instances, a POI may be an immune suppression factor. In some instances, a POI may be a proteolytically cleavable chimeric polypeptide as described herein, e.g., as used in a multi-component circuit as describe herein.

Certain nucleic acids and components thereof that may be adapted for use in the chimeric polypeptides and the methods and circuits described herein include but are not limited to e.g., those described in PCT Application No. US2016/019188 (Pub. No. WO 2016/138034), the disclosure of which is incorporated herein by reference in its entirety.

### Cells

The present disclosure includes cells engineered to express a chimeric polypeptide as described herein. In some instances, cells of the instant disclosure will include a nucleic acid encoding a chimeric polypeptide as described herein. In some instances, cells of the instant disclosure will include a nucleic acid operably linked to a transcription control element, e.g., a transcriptional activator, that is responsive the freed intracellular domain of chimeric polypeptide of the instant disclosure thereby inducing expression of the nucleic acid upon activation of the chimeric polypeptide. Any polypeptide of interest may be encoded from a nucleic acid within a cell operably linked to a transcription control element responsive to a chimeric polypeptide of the instant disclosure.

A method of the present disclosure can be used to modulate an activity of any eukaryotic cell. In some cases, the cell is in vivo. In some cases, the cell is ex vivo. In some cases, the cell is in vitro. In some cases, the cell is a mammalian cell. In some cases, the cell is a human cell. In some cases, the cell is a non-human primate cell. In some cases, the cell is rodent cell. In some cases, the cell is mouse cell. In some cases, the cell is a rat cell.

Suitable cells include neural cells; liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs)

In some cases, the stem cell is a hematopoietic stem cell (HSC), and the transcription factor induces differentiation of the HSC to differentiate into a red blood cell, a platelet, a lymphocyte, a monocyte, a neutrophil, a basophil, or an eosinophil. In some cases, the stem cell is a mesenchymal stem cell (MSC), and the transcription factor induces differentiation of the MSC into a connective tissue cell such as a cell of the bone, cartilage, smooth muscle, tendon, ligament, stroma, marrow, dermis, or fat.

Cells of the subject disclosure may be genetically modified host cells, e.g., modified with a nucleic acid of the present disclosure, i.e., host cells genetically modified with a nucleic acid comprising a nucleotide sequence encoding a chimeric polypeptide of the present disclosure. In one embodiment, the present disclosure provides a method of inducing expression of a heterologous polypeptide in a cell, e.g., a host cell genetically modified to contain a nucleic acid of the instant disclosure. The method generally involves contacting the cell with the binding partner of the specific binding member of a chimeric polypeptide of the present disclosure. Such binding induces cleavage of the Notch receptor polypeptide at the one or more proteolytic cleavage sites, thereby releasing the intracellular domain. Release of the intracellular domain may modulate an activity of the cell, e.g., induce expression of a heterologous gene or coding sequence.

In one embodiment, a method of the present disclosure generally involves contacting a cell with a peptide-MHC that binds the specific binding member of a chimeric polypeptide of the present disclosure. Such binding induces cleavage of the Notch receptor polypeptide at the one or more proteolytic cleavage sites, thereby releasing the intracellular domain. Release of the intracellular domain may modulate an activity of the cell, e.g., induce expression of a heterologous gene or coding sequence.

In some cases, the cell is a eukaryotic cell. In some cases, the cell is a mammalian cell, an amphibian cell, a reptile cell, an avian cell, or a plant cell. In some cases, the cell is a plant cell.

In some cases, the cell is a mammalian cell. In some cases, the cell is a human cell. In some cases, the cell is a mouse cell. In some cases, the cell is rat cell. In some cases, the cell is non-human primate cell. In some cases, the cell is lagomorph cell. In some cases, the cell is an ungulate cell.

In some cases, the cell is an immune cell, e.g., a T cell, a B cell, a macrophage, a dendritic cell, a natural killer cell, a monocyte, etc. In some cases, the cell is a T cell. In some cases, the cell is a cytotoxic T cell (e.g., a CD8+ T cell). In some cases, the cell is a helper T cell (e.g., a CD4+ T cell). In some cases, the cell is a regulatory T cell (“Treg”). In some cases, the cell is a B cell. In some cases, the cell is a macrophage. In some cases, the cell is a dendritic cell. In some cases, the cell is a peripheral blood mononuclear cell. In some cases, the cell is a monocyte. In some cases, the cell is a natural killer (NK) cell. In some cases, the cell is a CD4+, FOXP3+ Treg cell. In some cases, the cell is a CD4+, FOXP3 Treg cell.

In some instances, the cell is obtained from an individual. For example, in some cases, the cell is a primary cell. As another example, the cell is a stem cell or progenitor cell obtained from an individual.

As one non-limiting example, in some cases, the cell is an immune cell obtained from an individual. As an example, the cell can be a T lymphocyte obtained from an individual. As another example, the cell is a cytotoxic cell (e.g., a cytotoxic T cell, a helper T cell, etc.) obtained from an individual. As another example, the cell can be a helper T cell obtained from an individual. As another example, the cell can be a regulatory T cell obtained from an individual. As another example, the cell can be an NK cell obtained from an individual. As another example, the cell can be a macrophage obtained from an individual. As another example, the cell can be a dendritic cell obtained from an individual. As another example, the cell can be a B cell obtained from an individual. As another example, the cell can be a peripheral blood mononuclear cell obtained from an individual.

In some cases, the host cell is a somatic cell, e.g. a fibroblast, a hematopoietic cell, a neuron, a pancreatic cell, a muscle cell, a bone cell, a hepatocyte, a pancreatic cell, an epithelial cell, an endothelial cell, a cardiomyocyte, a T cell, a B cell, an osteocyte, and the like.

In some cases, the cell is genetically modified to express two or more different chimeric polypeptides of the present disclosure, including but not limited to e.g., 2 different chimeric polypeptides of the present disclosure, 3 different chimeric polypeptides of the present disclosure, 4 different chimeric polypeptides of the present disclosure, 5 different chimeric polypeptides of the present disclosure, etc.

Certain cells and components and activities thereof that may be adapted for use in the chimeric polypeptides and/or be modulated in the methods and circuits described herein include but are not limited to e.g., those described in PCT Application No. US2016/019188 (Pub. No. WO 2016/138034), the disclosure of which is incorporated herein by reference in its entirety.

### Circuits

The intracellular domain of a chimeric polypeptide of the present disclosure, when released upon binding of the binding partner to the specific binding member of the extracellular domain, may induce the expression of various polypeptides as described herein. In some instances, induced expression of two or more polypeptides may generate a logic gated circuit. Such logic gated circuits may include but are not limited to e.g., “AND gates”, “OR gates”, “NOT gates” and combinations thereof including e.g., higher order gates including e.g., higher order AND gates, higher order OR gates, higher order NOT gates, higher order combined gates (i.e., gates using some combination of AND, OR and/or NOT gates).

“AND” gates of the present disclosure include where two or more inputs are required for propagation of a signal. For example, in some instances, an AND gate allows signaling through a chimeric polypeptide of the instant disclosure and a second binding-dependent molecule. In an AND gate two inputs, e.g., two antigens, are required for signaling through the circuit.

“OR” gates of the present disclosure include where either of two or more inputs may allow for the propagation of a signal. For example, in some instances, an OR gate allows signaling through either of two different chimeric polypeptides of the instant disclosure. In an OR gate any one input, e.g., either of two antigens, may induce the signaling output of the circuit. In one embodiment, an OR gate may be achieved through the use of two separate molecules or constructs. In another embodiment, an OR gate may be achieved through the use of a single construct that recognizes two antigens, including e.g., a proteolytically cleavable chimeric polypeptide having two different specific binding members that each bind a different binding partner but either can activate the chimeric polypeptide. In some instances, an OR gate may be achieved through the use of a single construct that recognizes two antigens, including e.g., a proteolytically cleavable chimeric polypeptide having two different antibody specific binding members that each bind a different antigen but either antigen can activate the chimeric polypeptide.

“NOT” gates of the present disclosure include where an input is capable of preventing the propagation of a signal. For example, in some instances, a NOT gate inhibits signaling through a chimeric polypeptide of the instant disclosure. In one embodiment, a NOT gate may include the inhibition of a binding interaction. For example, a competitive inhibitor that prevents the binding of parts of a split chimeric polypeptide of the instant disclosure may serve as a NOT gate that prevents signaling through the circuit. In another embodiment, a NOT gate may include functional inhibition of an element of a circuit. For example, an inhibitor that functionally prevents signaling through a chimeric polypeptide of the instant disclosure or the outcome of signaling through a circuit may serve as a NOT gate.

In some instances, the production of immunosuppressive agents (e.g., an immune suppression factor) may provide NOT gate functionality in a multi-input circuit described herein.

Multi-input gates may make use of a NOT gate in various different ways to prevent signaling through some other component of a circuit or turn off a cellular response when and/or where a signal activating the NOT gate (e.g., a particular negative antigen) is present. For example, an AND+NOT gate may include a chimeric polypeptide of the instant disclosure that positively influences a particular cellular activity in the presence of a first antigen and a chimeric polypeptide of the instant disclosure that negatively influences the cellular activity in the presence of a second antigen.

In some instances, circuits of the present disclosure may make use of the recognition of intracellular antigens provided by specific binding members specific for peptide-MHC complexes displaying a peptide of an intracellular antigen. Circuits making use of intracellular antigen recognition may, in some instances, couple intracellular antigen recognition with recognition of a second antigen where the second antigen may also be an intracellular antigen or may be an extracellular (e.g., surface expressed) antigen. Accordingly, in some instances, a circuit may be an “inside-inside” circuit where the circuit relies upon the recognition of two intracellular antigens. In some instances, a circuit may be an “inside-outside” circuit where the circuit relies upon the first recognition of an intracellular antigen and the second recognition of an extracellular antigen. In some instances, a circuit may be an “outside-inside” circuit where the circuit relies upon the first recognition of an extracellular antigen and the second recognition of an intracellular antigen. In some instances, a circuit may be an “outside-outside” circuit where the circuit relies upon the recognition of two extracellular antigens. Such circuits are not limited to two antigens may, in some instances, include e.g., inside-inside-inside antigen circuits, inside-inside-outside antigen circuits, inside-outside-inside antigen circuits, outside-inside-inside antigen circuits, inside-outside-outside antigen circuits, etc.

In one embodiment, an “inside-inside” circuit may include a first chimeric polypeptide that, upon binding a first intracellular antigen in the context of a peptide-MHC, induces the expression of a therapeutic that recognizes a second intracellular antigen in the context of a peptide-MHC including, e.g., a TCR, a CAR, an antibody, a chimeric bispecific binding member, and the like. In one embodiment, an “inside-inside” circuit may include a first chimeric polypeptide that, upon binding a WT1 intracellular antigen in the context of MHC, induces the expression of a therapeutic that recognizes a second intracellular antigen in the context of a peptide-MHC including, e.g., a TCR, a CAR, an antibody, a chimeric bispecific binding member, and the like. In one embodiment, an “inside-inside” circuit may include a first chimeric polypeptide that, upon binding a NY-ESO1 intracellular antigen in the context of MHC, induces the expression of a therapeutic that recognizes a second intracellular antigen in the context of a peptide-MHC including, e.g., a TCR, a CAR, an antibody, a chimeric bispecific binding member, and the like.

In one embodiment, an “inside-outside” circuit may include a chimeric polypeptide that, upon binding an intracellular antigen in the context of a peptide-MHC, induces the expression of a therapeutic that recognizes an extracellular antigen including, e.g., a TCR, a CAR, an antibody, a chimeric bispecific binding member, and the like. In one embodiment, an “inside-outside” circuit may include a chimeric polypeptide that, upon binding a WT1 intracellular antigen in the context of MHC, induces the expression of a therapeutic that recognizes an extracellular antigen including, e.g., a TCR, a CAR, an antibody, a chimeric bispecific binding member, and the like. In one embodiment, an “inside-outside” circuit may include a chimeric polypeptide that, upon binding a NY-ESO1 intracellular antigen in the context of MHC, induces the expression of a therapeutic that recognizes an extracellular antigen including, e.g., a TCR, a CAR, an antibody, a chimeric bispecific binding member, and the like.

Multi-input circuits and logic gated systems of the instant disclosure are not limited to those specifically described and may include alternative configurations and/or higher order gates as compared to those described. For example, in some instances a logic gated system of the instant disclosure may be a two input gate, a three input gate, a four input gate, a five input gate, a six input gate, a seven input gate, an eight input gate, a nine input gate, a ten input gate or greater. Any construct described herein including e.g., a chimeric polypeptide, a CAR, a TCR, a chimeric bispecific binding member, and the may find use in a circuit in conjunction with any other construct described herein including e.g., a chimeric polypeptide, a CAR, a TCR, a chimeric bispecific binding member, a second chimeric polypeptide, a second CAR, a second TCR, a second chimeric bispecific binding member, and the like.

Certain circuits and components thereof that may be adapted for use with the chimeric polypeptides and the methods described herein include but are not limited to e.g., those described in PCT Application No. US2016/019188 (Pub. No. WO 2016/138034), the disclosure of which is incorporated herein by reference in its entirety.

### Kits

The present disclosure provides a kit for carrying out a method as described herein and/or constructing one or more chimeric polypeptides, nucleic acids encoding chimeric polypeptides, components thereof, etc.

In some cases, a subject kit comprises an expression vector comprising a nucleotide sequence encoding a chimeric polypeptide of the present disclosure or one or more portions thereof. In some cases, a subject kit comprises a chimeric polypeptide of the present disclosure.

In some cases, a subject kit comprises a cell, e.g., a host cell or host cell line, that is or is to be genetically modified with a nucleic acid comprising a nucleotide sequence encoding a chimeric polypeptide of the present disclosure. In some cases, a subject kit comprises a cell, e.g., a host cell, that is or is to be genetically modified with a recombinant expression vector comprising a nucleotide sequence encoding a chimeric polypeptide of the present disclosure. Kit components can be in the same container, or in separate containers.

Any of the above-described kits can further include one or more additional reagents, where such additional reagents can be selected from: a dilution buffer; a reconstitution solution; a wash buffer; a control reagent; a control expression vector; a negative control polypeptide (e.g., a chimeric polypeptide that lacks the one or more proteolytic cleavage sites, such that, upon binding, the intracellular domain is not released); a positive control polypeptide; a reagent for in vitro production of the chimeric polypeptide, and the like.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered as below are provided. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A chimeric polypeptide comprising, from N-terminal to C-terminal and in covalent linkage:
   a) an extracellular domain comprising a specific binding member that specifically binds to a peptide-major histocompatibility complex (peptide-MHC);
   b) a proteolytically cleavable Notch receptor polypeptide comprising one or more proteolytic cleavage sites; and
   c) an intracellular domain comprising a transcriptional activator, wherein binding of the specific binding member to the peptide-MHC induces cleavage of the Notch receptor polypeptide at the one or more proteolytic cleavage sites, thereby releasing the intracellular domain.
2. The chimeric polypeptide of Aspect 1, wherein the specific binding member comprises an antibody.
3. The chimeric polypeptide of Aspect 2, wherein the antibody is a nanobody, a diabody, a triabody, or a minibody, a F(ab′)₂ fragment, a Fab fragment, a single chain variable fragment (scFv) or a single domain antibody (sdAb).
4. The chimeric polypeptide of any of the preceding aspects, wherein the specific binding member specifically binds a peptide-MHC comprising an intracellular cancer antigen peptide.
5. The chimeric polypeptide of Aspect 4, wherein the intracellular cancer antigen peptide is a WT1 peptide or a NY-ESO peptide.
6. The chimeric polypeptide of any of the preceding aspects, wherein the Notch receptor polypeptide comprises, at its N-terminus, one or more epidermal growth factor (EGF) repeats.
7. The chimeric polypeptide of Aspect 6, wherein the Notch receptor polypeptide comprises, at its N-terminus, 2 to 11 EGF repeats.
8. The chimeric polypeptide of any of the preceding aspects, wherein the Notch receptor polypeptide comprises a synthetic linker.
9. The chimeric polypeptide of Aspect 8, wherein the Notch receptor polypeptide comprises a synthetic linker between the one or more EGF repeats and the one or more proteolytic cleavage sites.
10. The chimeric polypeptide of any of the preceding aspects, wherein the Notch receptor polypeptide has a length from 50 amino acids to 1000 amino acids.
11 The chimeric polypeptide of Aspect 10, wherein the Notch receptor polypeptide has a length from 300 amino acids to 400 amino acids.
12. The chimeric polypeptide of any of the preceding aspects, wherein the one or more proteolytic cleavage sites comprises an S2 proteolytic cleavage site, an S3 proteolytic cleavage site or a combination thereof.
13. The chimeric polypeptide of Aspect 12, wherein the one or more proteolytic cleavage sites comprises an S2 proteolytic cleavage site that is an ADAM-17-type protease cleavage site comprising an Ala-Val dipeptide sequence.
14. The chimeric polypeptide of any of the preceding aspects, wherein the one or more proteolytic cleavage sites comprises an S3 proteolytic cleavage site that is a gamma-secretase (γ-secretase) cleavage site comprising a Gly-Val dipeptide sequence.
15. The chimeric polypeptide of any of the preceding aspects, wherein the one or more proteolytic cleavage sites further comprises an S1 proteolytic cleavage site.
16. The chimeric polypeptide of Aspect 15, wherein the S1 proteolytic cleavage site is a furin-like protease cleavage site comprising the amino acid sequence Arg-X-(Arg/Lys)-Arg (SEQ ID NO:130), where X is any amino acid.
17. The chimeric polypeptide of any of the preceding aspects, wherein the Notch receptor polypeptide lacks an S1 proteolytic cleavage site.
18. The chimeric polypeptide of any of the preceding aspects, wherein the Notch receptor polypeptide has at least 85% amino acid sequence identity to a sequence provided in FIG. 31A-32B.
19. The chimeric polypeptide of Aspect 18, wherein the Notch receptor polypeptide has at least 85% amino acid sequence identity to the sequence provided in FIG. 31A or the sequence provided in FIG. 31B.
20. A nucleic acid encoding the chimeric polypeptide according to any of Aspects 1-19.
21. The nucleic acid of Aspect 20, wherein the nucleic acid further comprises a transcriptional control element responsive to the transcriptional activator operably linked to a nucleic acid sequence encoding a polypeptide of interest (POI).
22. The nucleic acid of Aspect 21, wherein the POI is a heterologous polypeptide selected from the group consisting of: a reporter protein, a chimeric antigen receptor (CAR), an antibody, a chimeric bispecific binding member, an engineered T cell receptor (TCR) and an innate-immune response inducer.
23. A recombinant expression vector comprising the nucleic acid according to any of Aspects 20-22.
24. A method of inducing expression of a heterologous polypeptide in a cell, the method comprising: contacting a cell with a peptide-major histocompatibility complex (peptide-MHC), wherein the cell expresses a chimeric polypeptide according to any of Aspects 1-19 and comprises a sequence encoding the heterologous polypeptide operably linked to a transcriptional control element responsive to the transcriptional activator of the chimeric polypeptide, thereby releasing the intracellular domain of the chimeric polypeptide and inducing expression of the heterologous polypeptide.
25. The method according to Aspect 24, wherein the heterologous polypeptide is a reporter protein, a chimeric antigen receptor (CAR), an antibody, a chimeric bispecific binding member, an engineered T cell receptor (TCR) and an innate-immune response inducer.
26. A host cell comprising:
   a) a nucleic acid encoding a chimeric polypeptide according to any of Aspects 20-22 that specifically binds to a first peptide-major histocompatibility complex (peptide-MHC); and
   b) a transcriptional control element responsive to the transcriptional activator of the chimeric polypeptide operably linked to a nucleic acid encoding a polypeptide of interest (POI).
27. The host cell of Aspect 26, wherein the host cell is genetically modified and the nucleic acid and the transcriptional control element are present within the genome of the host cell.
28. The host cell of Aspect 26, wherein the nucleic acid and the transcriptional control element are present extrachromosomally within the host cell.
29. The host cell of any of Aspects 26-28, wherein the POI is a heterologous polypeptide.
30. The host cell of Aspect 29, wherein the heterologous polypeptide is selected from the group consisting of: a reporter protein, a chimeric antigen receptor (CAR), an antibody, a chimeric bispecific binding member, an engineered T cell receptor (TCR) and an innate-immune response inducer.
31. The host cell of Aspect 30, wherein the heterologous polypeptide is a CAR that specifically binds to a second peptide-MHC.
32. The host cell of Aspect 31, wherein the specific binding member of the chimeric polypeptide specifically binds to a first peptide-MHC comprising a first intracellular cancer antigen peptide and the CAR specifically binds to a second peptide-MHC comprising a second intracellular cancer antigen peptide.
33. The host cell of Aspect 32, wherein the first intracellular cancer antigen peptide is a WT1 peptide and the second intracellular cancer antigen peptide is a NY-ESO peptide.
34. The host cell of Aspect 32, wherein the first intracellular cancer antigen peptide is a NY-ESO peptide and the second intracellular cancer antigen peptide is a WT1 peptide.
35. The host cell of Aspect 30, wherein the heterologous polypeptide is an engineered TCR that specifically binds to a second peptide-MHC.
36. The host cell of Aspect 35, wherein the specific binding member of the chimeric polypeptide specifically binds to a first peptide-MHC comprising a first intracellular cancer antigen peptide and the engineered TCR specifically binds to a second peptide-MHC comprising a second intracellular cancer antigen peptide.
37. The host cell of Aspect 36, wherein the first intracellular cancer antigen peptide is a WT1 peptide and the second intracellular cancer antigen peptide is a NY-ESO peptide.
38. The host cell of Aspect 36, wherein the first intracellular cancer antigen peptide is a NY-ESO peptide and the second intracellular cancer antigen peptide is a WT1 peptide.
39. A host cell comprising:
   a) a nucleic acid encoding a chimeric polypeptide comprising, from N-terminal to C-terminal and in covalent linkage:
      i) an extracellular domain comprising a specific binding member that specifically binds to a target molecule present on the surface of a cancer cell;
      ii) a proteolytically cleavable Notch receptor polypeptide comprising one or more proteolytic cleavage sites; and
      iii) an intracellular domain comprising a transcriptional activator;
   b) a nucleic acid encoding a chimeric bispecific binding member operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the chimeric bispecific binding member to be expressed.
40. The host cell according to Aspect 39, wherein the chimeric bispecific binding member comprises a binding domain specific for a cancer antigen and a binding domain specific for a protein expressed on the surface of an immune cell.
41. The host cell according to Aspect 39 or 40, wherein the chimeric bispecific binding member comprises at least one antibody derived antigen-binding domains.
42. The host cell according to Aspect 41, wherein the chimeric bispecific binding member is a bispecific antibody or a fragment thereof.
43. The host cell according to any of Aspects 39-41, wherein the chimeric bispecific binding member comprises at least one receptor or ligand binding domain of a ligand-receptor binding pair.
44. The host cell according to any of Aspects 39-43, wherein the chimeric bispecific binding member comprises at least one antibody derived antigen-binding domain and at least one receptor or ligand binding domain of a ligand-receptor binding pair.
45. The host cell according to any of Aspects 40-44, wherein the protein expressed on the surface of an immune cell is CD3.
46. The host cell according to any of Aspects 40-44, wherein the protein expressed on the surface of an immune cell is Natural Killer Group 2D (NKG2D) receptor.
47. The host cell according to any of Aspects 39-46, wherein the target molecule is a cancer antigen.
48. The host cell according to any of Aspects 39-46, wherein the target molecule is a tissue specific molecule.
49 The host cell according to any of Aspects 39-46, wherein the target molecule is an organ specific molecule.
50. The host cell according to any of Aspects 39-46, wherein the target molecule is a cell type specific molecule.
51. A method of treating a subject for a neoplasia comprising administering to the subject an effective amount of host cells according to any of Aspects 39-50, wherein the neoplasia expresses the target molecule and the cancer antigen.
52. A host cell comprising:
   a) a nucleic acid encoding a chimeric polypeptide comprising, from N-terminal to C-terminal and in covalent linkage:
      i) an extracellular domain comprising a specific binding member that specifically binds to a target molecule present on the surface of a cancer cell;
      ii) a proteolytically cleavable Notch receptor polypeptide comprising one or more proteolytic cleavage sites; and
      iii) an intracellular domain comprising a transcriptional activator;
   b) a nucleic acid encoding an anti-Fc chimeric antigen receptor (CAR) operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the anti-Fc CAR to be expressed.
53. The host cell of Aspect 52, wherein the target molecule is a cancer antigen.
54. The host cell of Aspect 52, wherein the target molecule is a tissue specific molecule.
55. The host cell of Aspect 52, wherein the target molecule is an organ specific molecule.
56. The host cell of Aspect 52, wherein the target molecule is a cell type specific molecule.
57. The host cell of any of Aspects 52-56, wherein the host cell further comprises a nucleic acid encoding an antibody specific for a cancer antigen present on the surface of a cancer cell and comprising an Fc region that is bound by the anti-Fc CAR.
58. The host cell of Aspect 57, wherein the nucleic acid encoding the antibody is operably linked to the transcriptional control element.
59. A method of treating a subject for a neoplasia comprising administering to the subject an effective amount of host cells according to any of Aspects 52-58, wherein the neoplasia expresses the target molecule.
60. The method of Aspect 59, wherein the method further comprises administering to the subject an antibody specific for a cancer antigen present on the surface of a cancer cell and comprising an Fc region that is bound by the anti-Fc CAR.
61. A host cell comprising:
   a) a nucleic acid encoding a chimeric polypeptide comprising, from N-terminal to C-terminal and in covalent linkage:
      i) an extracellular domain comprising a specific binding member that specifically binds to a target molecule present on the surface of a cancer cell;
      ii) a proteolytically cleavable Notch receptor polypeptide comprising one or more proteolytic cleavage sites; and
      iii) an intracellular domain comprising a transcriptional activator;
   b) a nucleic acid encoding an innate-immune response inducer operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the innate-immune response inducer to be expressed.
62. The method of Aspect 61, wherein the target molecule is a tissue specific molecule.
63. The method of Aspect 61, wherein the target molecule is an organ specific molecule.
64. The method of Aspect 61, wherein the target molecule is a cell type specific molecule.
65. The method of Aspect 61, wherein the target molecule is a cancer antigen.
66. The method of any of Aspects 61-65, wherein the innate-immune response inducer is bacterial protein or fragment thereof.
67. The method of any of Aspects 61-65, wherein the innate-immune response inducer is viral protein or fragment thereof.
68. The method of any of Aspects 61-65, wherein the innate-immune response inducer is fungal protein or fragment thereof.
69. The method of any of Aspects 61-68, wherein the innate-immune response inducer is a protein or fragment thereof expressed by a mammalian parasite.
70. The method of Aspect 69, wherein the mammalian parasite is a human parasite.
71. A method of inducing a local innate immune response in an area of a subject, the method comprising administering to the subject an effective amount of host cells according to any of Aspects 61-70, wherein the area expresses the target molecule.
72. The method of Aspect 71, wherein the area of the subject comprises a neoplasia.
73. A host cell comprising:
   a) a nucleic acid encoding a chimeric polypeptide comprising, from N-terminal to C-terminal and in covalent linkage:
      i) an extracellular domain comprising a specific binding member that specifically binds to a target molecule present on the surface of a cancer cell;
      ii) a proteolytically cleavable Notch receptor polypeptide comprising one or more proteolytic cleavage sites; and
      iii) an intracellular domain comprising a transcriptional activator;
   b) a nucleic acid encoding an immune suppression factor operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the immune suppression factor to be expressed.
74. The method of Aspect 73, wherein the target molecule is a tissue specific molecule.
75. The method of Aspect 73, wherein the target molecule is an organ specific molecule.
76. The method of Aspect 73, wherein the target molecule is a cell type specific molecule.
77. The method of Aspect 73, wherein the target molecule is an autoantigen.
78. The method of any of Aspects 73-77, wherein the immune suppression factor is an immunosuppressive cytokine.
79. The method of Aspects 78, wherein the immunosuppressive cytokine is IL-10.
80. The method of any of Aspects 73-77, wherein the immune suppression factor is a cell-to-cell signaling immunosuppressive ligand.
81. The method of Aspects 80, wherein the cell-to-cell signaling immunosuppressive ligand is PD-L1.
82. A method of suppressing an immune response in a subject, the method comprising administering to the subject an effective amount of host cells according to any of Aspects 73-81, wherein the subject expresses the target molecule.
83. The method of Aspect 82, wherein the subject has an autoimmune disease.
84. A method of killing a heterogeneous tumor, the method comprising:
   contacting a heterogeneous tumor comprising a first cell expressing a killing antigen and a second cell expressing the killing antigen and a priming antigen with an engineered immune cell comprising:
   a proteolytically cleavable chimeric polypeptide that specifically binds the priming antigen;
   a nucleic acid sequence encoding a therapeutic polypeptide that specifically binds the killing antigen; and
   a transcriptional control element operably linked to the nucleic acid that is responsive to the proteolytically cleavable chimeric polypeptide,
   wherein binding of the proteolytically cleavable chimeric polypeptide to the priming antigen activates the transcriptional control element to induce expression of the therapeutic polypeptide which, when bound to the killing antigen, kills the first and second cells of the heterogeneous tumor.
85. The method of Aspect 84, wherein the therapeutic polypeptide is a chimeric antigen receptor (CAR).
86. The method of Aspect 84, wherein the therapeutic polypeptide is a T cell Receptor (TCR).
87. The method of Aspect 84, wherein the therapeutic polypeptide is a therapeutic antibody.
88. The method of Aspect 84, wherein the therapeutic polypeptide is a chimeric bispecific binding member.
89. The method of any of Aspects 84-88, wherein at least one of the priming antigen or the killing antigen is an intracellular antigen presented in the context of MHC.
90. The method of any of Aspects 84-89, wherein both the priming antigen and the killing antigen are intracellular antigens presented in the context of MHC.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Peptide-Major Histocompatibility Complex (Peptide-MHC) Induced Signaling

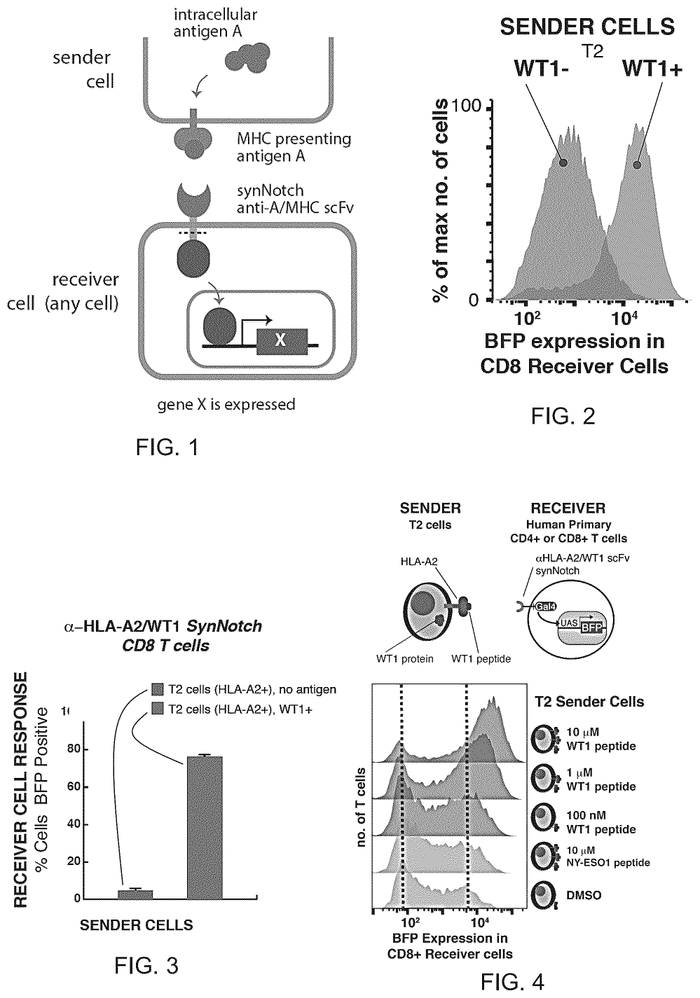

The ability to affect cellular signaling through recognition of a peptide-MHC by a cleavable chimeric Notch polypeptide was investigated. As diagramed in FIG. 1, chimeric polypeptides were designed that include a cleavable Notch polypeptide linked to an extracellular domain that includes a specific binding member that specifically binds to a peptide-MHC of an intracellular antigen of a “sender cell”. The extracellular domain of a chimeric polypeptide expressed by a “receiver cell” binds the peptide-MHC of the “sender cell”. Upon such binding, the cleavable Notch polypeptide is cleaved releasing the intracellular portion which can affect intracellular processes including e.g., the expression of a transgene (e.g., “X” in FIG. 1) operatively linked to a promoter that is responsive to the freed intracellular domain.

As a representative example, a chimeric polypeptide was designed with an extracellular domain that binds to a Wilms Tumor Protein 1 (WT1) peptide-MHC and a GAL4 transcription activator intracellular domain that, when freed, binds an Upstream Activation Sequence (UAS) operatively linked to a Blue Fluorescent Protein (BFP) to serve as an activation reporter when the system is engineered into CD8 receiver cells. FIG. 2 demonstrates the relative levels of receiver cells expressing BFP reporter when contacted with either peptide-MHC negative (WT1−) or positive (WT1+) T2 sender cells. FIG. 3 provides quantification of receiver CD8 T cell response assayed in the presence of T2 cells expressing HLA-A2 with or without target WT1 intracellular antigen expression. As can be seen in FIG. 2 and FIG. 3 T cell response is highly specific to and dependent upon the presence of the presented intracellular antigen.

As demonstrated in FIG. 4, receiver cell response is also dependent on the amount of presented intracellular antigen. T2 sender cells were engineered to express different amounts of WT1 peptide-MHC (10 μM, 1 μM and 100 nM) and receiver cell response was assayed with each population. Reporter expression was seen to correlate to WT1 peptide-MHC expression level indicating dose dependent activation.

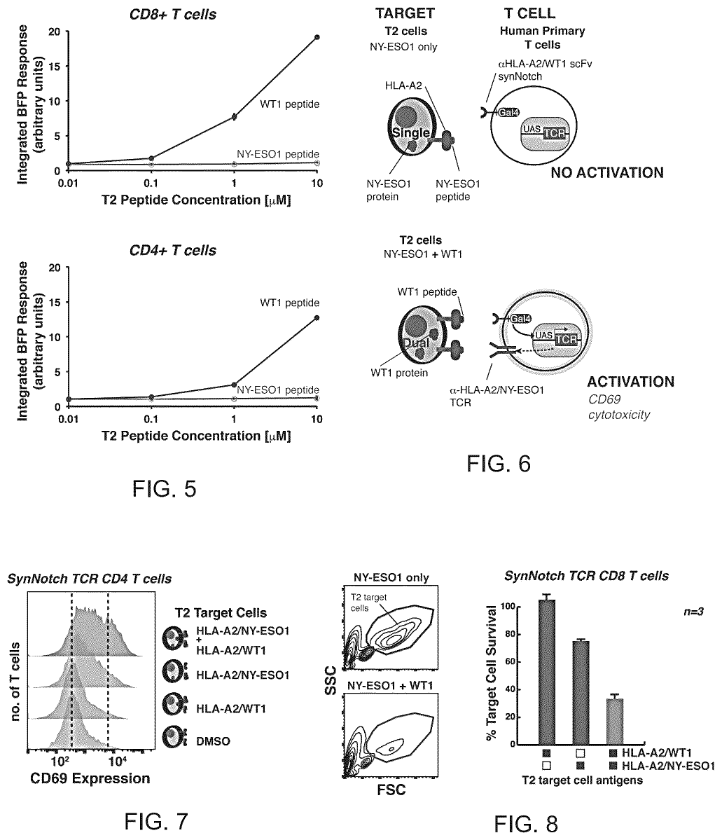

In addition, when assayed in the presence of irrelevant intracellular antigen peptide-MHC (NY-ESO1 peptide) expressing T2 cells reporter activation was similar to negative control (DMSO), further indicating that activation is antigen-specific. Across three different concentrations of either target intracellular antigen WT1 peptide-MHC or irrelevant intracellular antigen NY-ESO1 peptide-MHC, antigen concentration dependent reporter activation was seen in receiver cells in response to WT1 but not NY-ESO1 in both CD8+ T cells and CD4+ T cells (FIG. 5).

### Example 2: Dual Intracellular Antigen Dependent T Cell Activation

A dual intracellular antigen AND-gate was designed such that engineered T cell activation would be dependent upon the presentation of two intracellular antigens on a target cell. Specifically, as a representative example as presented in FIG. 6, a system was designed and engineered into T cells where expression of an anti-HLA-A2/NY-ESO1 TCR is induced by the freed intracellular domain of an anti-HLA-A2/WT1 scFv synNotch. When presented with a T2 target cell expressing NY-ESO1 only, expression of the anti-HLA-A2/NY-ESO1 TCR is not induced and no T cell activation occurs. However, when presented with a T2 target cell expressing both NY-ESO1 and WT1, binding of WT1 peptide-MHC to the anti-HLA-A2/WT1 scFV synNotch induces cleavage of the Notch polypeptide, releasing a GAL4 transcription activator which binds a UAS driving expression of the anti-HLA-A2/NY-ESO1 TCR. Binding of the expressed anti-HLA-A2/NY-ESO1 TCR to the NY-ESO1 peptide-MHC on the target cell causes activation of the T cell, e.g., as detected using CD69 and/or cytotoxicity assays.

T cell activation in such a system was tested using T2 target cells expressing either antigen or both. As can be seen in FIG. 7, robust activation, as measured using CD69 expression, was seen when the engineered T cells were contacted with dual antigen (HLA-A2/NY-ESO1 and HLA-A2/WT1) T2 target cells. However, an absence of activation, similar to negative control (DMSO), was seen when the engineered T cells were contacted with T2 cells expressing only one of the two intracellular antigens (HLA-A2/NY-ESO1 or HLA-A2/WT1). Corresponding results for T2 cell killing were seen when the engineered T cells were presented with either dual- or single-antigen expressing T2 target cells (FIG. 8). These results demonstrate the functionality of dual-intracellular antigen AND-gates and the specificity of such in activating engineered T cells only in response to target cells expressing both target intracellular antigens.

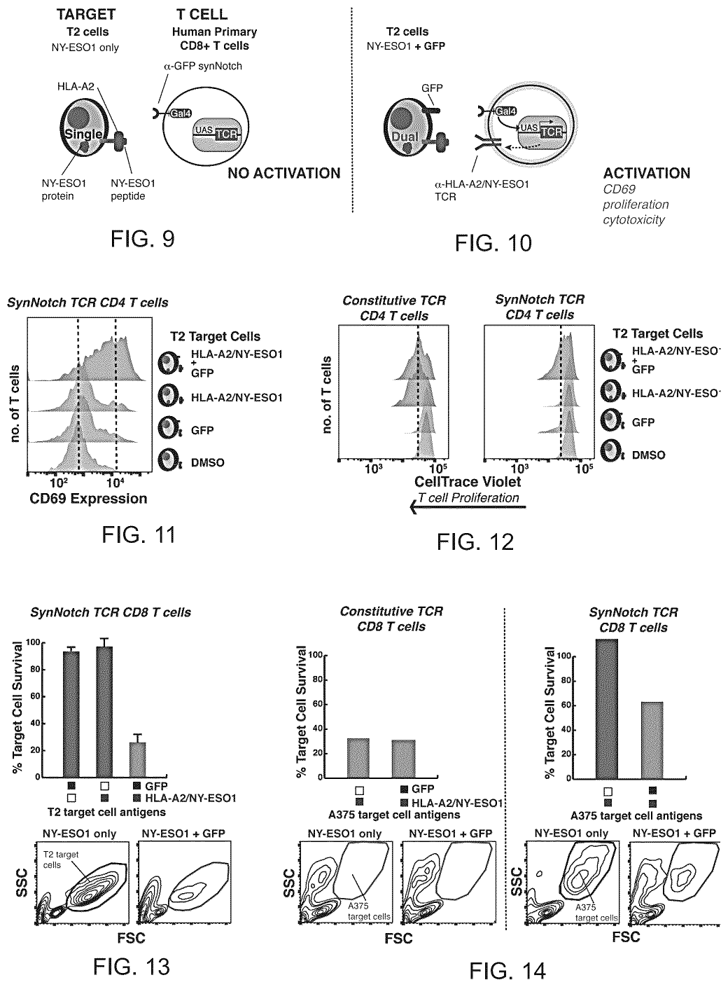

To further demonstrate the versatility of this approach, an additional dual-antigen AND-gate was designed and tested targeting cells expressing both a surface target antigen and an intracellular target antigen. Specifically, as a representative example as presented in FIG. 9 and FIG. 10, a system was designed and engineered into T cells where expression of an anti-HLA-A2/NY-ESO1 TCR is induced by the freed intracellular domain of an anti-surfaced-expressed-GFP synNotch. When presented with a T2 target cell expressing NY-ESO1 only, expression of the anti-HLA-A2/NY-ESO1 TCR is not induced and no T cell activation occurs. However, when presented with a T2 target cell expressing both NY-ESO1 and GFP, binding of surface-expressed-GFP to the anti-GFP synNotch induces cleavage of the Notch polypeptide, releasing a GAL4 transcription activator which binds a UAS driving expression of the anti-HLA-A2/NY-ESO1 TCR. Binding of the expressed anti-HLA-A2/NY-ESO1 TCR to the NY-ESO1 peptide-MHC on the target cell causes activation of the T cell.

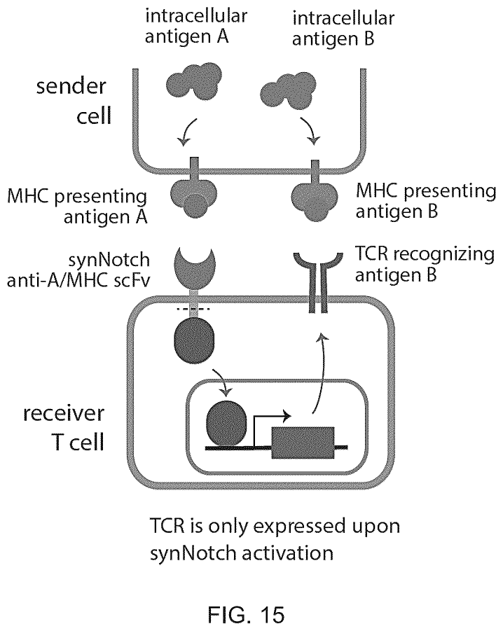

Activation of engineered CD4+ and CD8+ T cells expressing the dual-antigen circuit was assayed using CD69 expression (FIG. 11), T cell proliferation (FIG. 12), T2 target cell killing (FIG. 13) and A375 target cell killing (FIG. 14). In each assay T cell activation and downstream effects, such as target cell killing, was dependent on expression of both target antigens by the target cells, demonstrating the robust specificity of dual-antigen synNotch AND-gates regardless of whether a combination of intracellular antigens are used (e.g., as diagramed generally in FIG. 15) or surface expressed antigen responsive components are combined with one or more intracellular antigen recognizing elements.

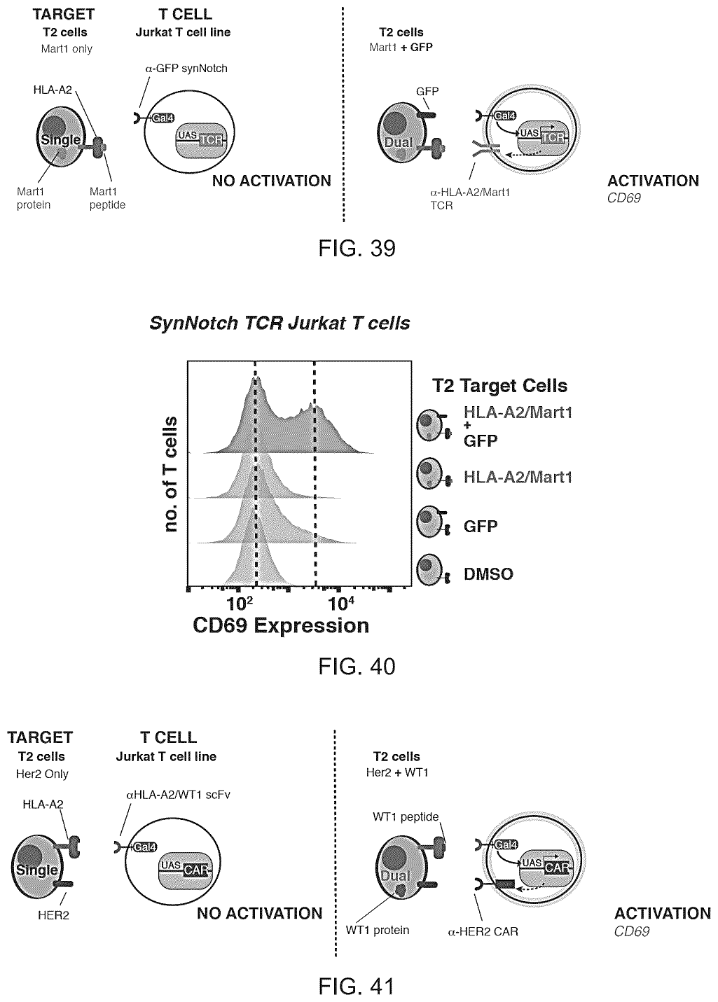

To further demonstrate the versatility of this approach, additional dual-antigen AND-gates were designed and tested targeting cells expressing both a surface target antigen and an intracellular target antigen other than NY-ESO1. Specifically, as schematically depicted in FIG. 39, a system was designed and engineered into T cells where expression of an anti-HLA-A2/Mart1 is induced by the freed intracellular domain of an anti-surfaced-expressed-GFP synNotch. When presented with a T2 target cell expressing Mart1 only, expression of the anti-HLA-A2/Mart1 TCR is not induced and no T cell activation occurs. However, when presented with a T2 target cell expressing both Mart1 and GFP, binding of surface-expressed-GFP to the anti-GFP synNotch induces cleavage of the Notch polypeptide, releasing a GAL4 transcription activator which binds a UAS driving expression of the anti-HLA-A2/Mart1 TCR. Binding of the expressed anti-HLA-A2/Mart1 TCR to the Mart1 peptide-MHC on the target cell causes activation of the T cell. A Jurkat T cell line was engineered to express this dual-antigen circuit, and activation of the T cells was assayed using CD69 expression. As provided in FIG. 40, the results of the assay showed that T cell activation, as measured by CD69 upregulation, was dependent on the expression of both target antigens by the target cells, thus further demonstrating the robust specificity of dual-antigen synNotch AND-gates targeting pMHC antigens.

### Example 3: Specific Local Delivery of Chimeric Bispecific Binding Members

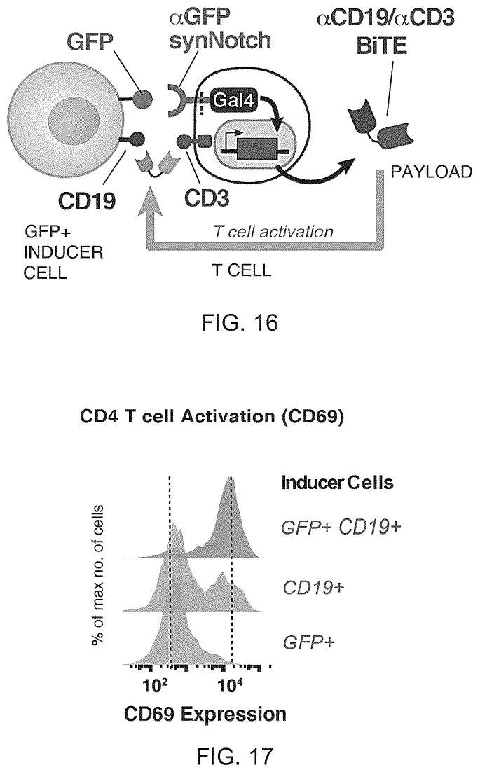

The use of synNotch to induce specific local expression of chimeric bispecific binding members was assayed using a representative two component system. Specifically, as depicted in FIG. 16, an anti-GFP synNotch was used to drive expression of an anti-CD19/anti-CD3 bispecific T cell engager (BiTE) (Blinatumomab) in an engineered T cell. Upon binding GFP expressed on the surface of an inducer target cell, the anti-GFP synNotch releases a GAL4 transcriptional activator which binds a UAS operably linked to sequence encoding the anti-CD19/anti-CD3 BiTE. Expression of the BiTE results in dual-binding of target cell expressed CD19 and T cell expressed CD3 and T cell activation.

CD4+ T cell activation was assayed using CD69 expression. CD4+ T cells expressing an anti-GFP-GAL4 synNotch and having sequence encoding an anti-CD19/anti-CD3 BiTE operably linked to a GAL4 responsive UAS were contacted with inducer cells expressing CD19 only, GFP only or both CD19 and GFP. Significant T cell activation was seen only when inducer cells expressing both CD19 and GFP were used (FIG. 17). These in vitro results demonstrate that a chimeric bispecific binding member payload can be effectively and specifically expressed using a synNotch gated circuit and used to control cellular responses such as, e.g., T cell activation.

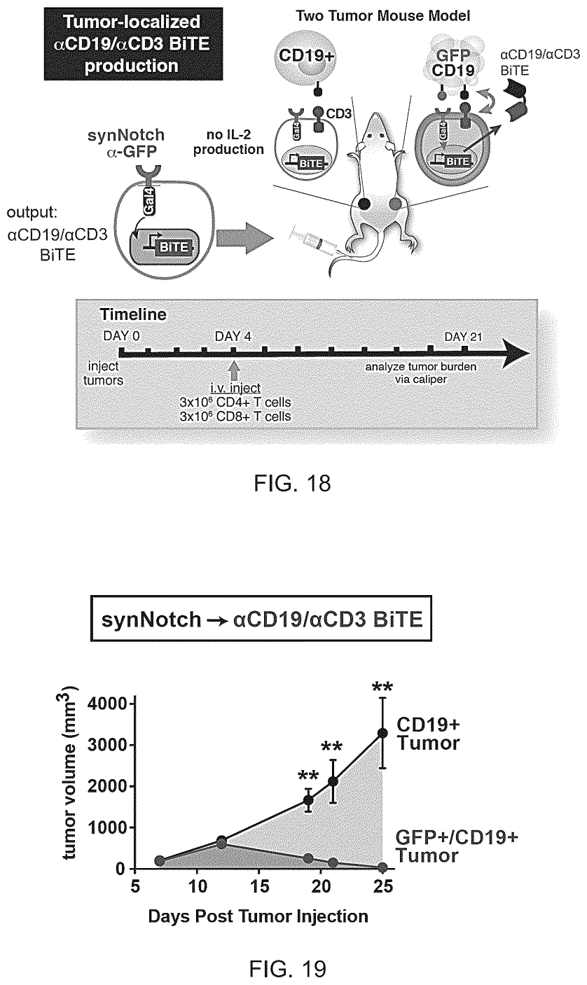

The use of synNotch gated expression of a chimeric bispecific binding member was further investigated in vivo, using, as a representative example, a model of tumor-localized α-CD19/α-CD3 BiTE production (FIG. 18). Mice were subcutaneously injected with priming antigen negative (GFP−) target antigen positive (CD19+) tumor cells in the left flank and dual positive (GFP+/CD19+) tumor cells in the right flank. α-GFP synNotch T cells (CD4+ and CD8+) in control of α-CD19/α-CD3 bispecific T cell engager (BiTE) expression were injected into the mice after tumors were established. Tumors were harvested at the indicated timepoints to analyze tumor burden by caliper measurement.

In the described model tumor-localized production of α-CD19/α-CD3 BiTE was seen to effectively reduce tumor burden in a priming antigen-dependent manner (FIG. 19). Tumor volume was measured for left flank and right flank tumors as described above in relation to FIG. 18. Over the course of treatment tumor volume was reduced in tumors expressing both the priming antigen (GFP) and the BiTE targeted antigen (CD19) (GFP+/CD19+ Tumor). In comparison, tumor volume was not reduced in the right flank tumor expressing the BiTE targeted antigen but not the priming antigen (CD19+ Tumor). These results demonstrate the effective use of a two antigen T cell AND-gate to drive spatial specific targeting of a BiTE payload to effectively reduce tumor volume in vivo. The α-GFP synNotch could be replaced with any other SynNotch targeting effectively any other antigen, endogenously or heterologously expressed, for tissue specific, organ specific or cell type specific T cell priming.

### Example 4: Specific Induction of Innate Immune Response

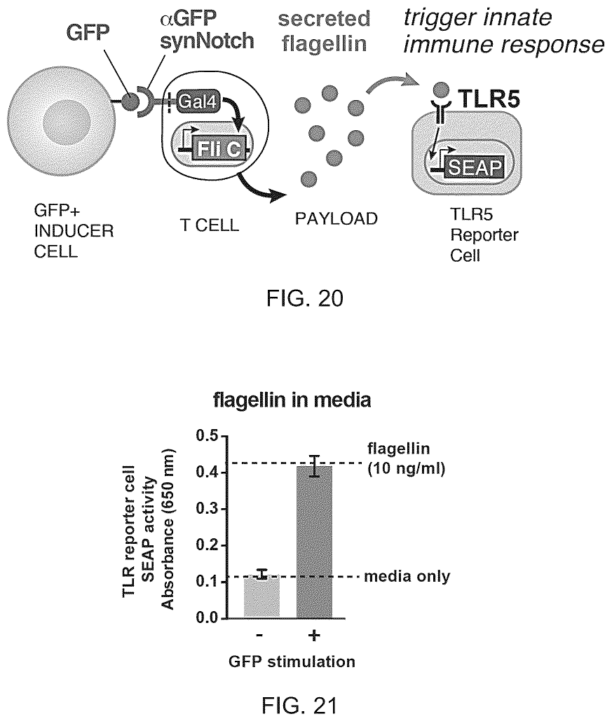

The use of synNotch to specifically and/or locally induce innate immune response was investigated using a representative model system. Specifically, as depicted in FIG. 20, a system was devised using a synNotch specific for surface expressed GFP to drive expression of a reporter operably linked to nucleic acid sequence encoding flagellin (FliC) in an engineered T cell. Secretion of the flagellin payload from the T cell is dependent on synNotch binding GFP present on the surface of an inducer cell. To detect and measure innate immune response induction a Secreted Alkaline Phosphatase (SEAP) reporter was used where binding of flagellin to Toll-like receptor 5 (TLR5) on the surface of a TLR5 reporter cell drives expression of SEAP allowing quantification of innate immune response activity.

T cells engineered with the above described system were cultured in the presence of TLR5 reporter cells. TLR reporter cell SEAP activity was measured by absorbance at 650 nm with or without GFP stimulation using surface-expressing GFP+ inducer cells. The results of this assay, presented in FIG. 21, showed that the innate immune response was specifically activated in a GFP dependent manner. This example demonstrates that synNotch can be used to specifically and/or locally induce an innate immune response by driving the expression of an innate immune response inducing polypeptide using an antigen dependent synNotch polypeptide.

### Example 5: Treating Heterogeneous Tumors

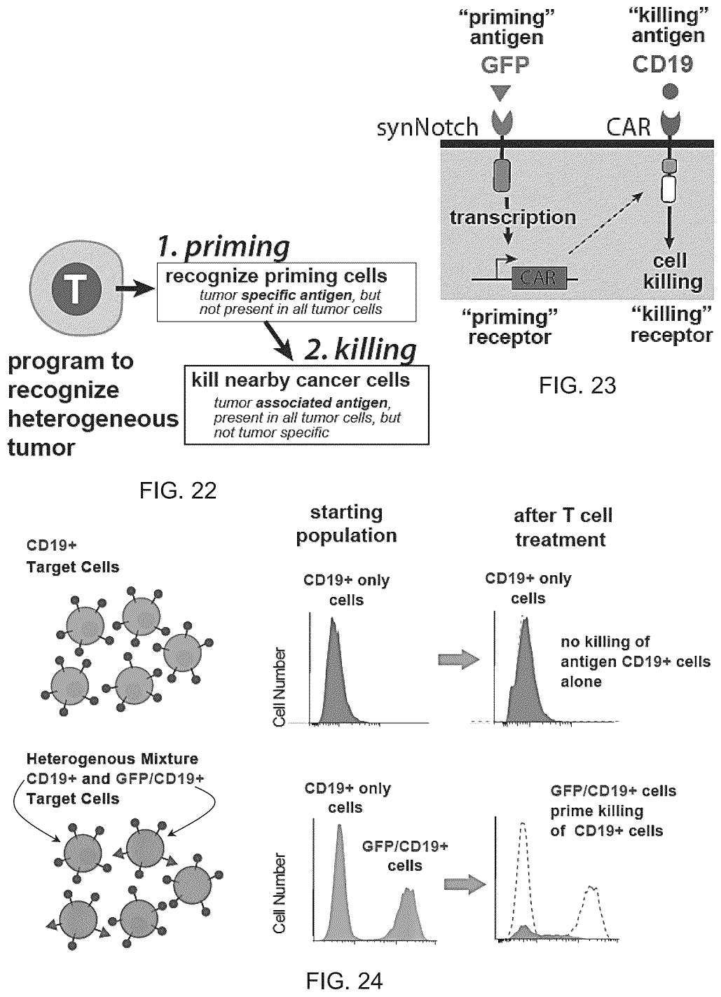

A multi-component strategy was developed, as depicted in FIG. 22, to program a T cell to recognize a heterogeneous tumor. The T cell is programed to (1) recognize priming cells present in the heterogeneous tumor using a tumor specific antigen that is specific to the tumor but not present in all tumor cells and (2) kill nearby cells of the heterogeneous tumor using a tumor associated antigen that is present in all tumor cells but is not tumor specific.

The use of a synNotch system to treat a heterogeneous tumor was investigated using a representative model system. Specifically, as depicted in FIG. 23, a T cells was engineered to produce a “priming” synNotch receptor specific for a GFP priming antigen and a “killing” CAR specific for CD19. The system was designed such that when the synNotch priming receptor binds the GFP priming antigen the freed intracellular domain of the synNotch drives expression of the CD19-specific killing CAR, resulting in cell killing when CD19 is present.

As depicted in FIG. 24, when a population of target cells expressing CD19 but not the GFP priming antigen were treated with the engineered T cells no killing of the target cells was observed. When a heterogeneous population of cells that included dual-expressing CD19+/GFP+ target cells as well as CD19+/GFP-target cells was treated with the engineered T cells, killing of the both the CD19+/GFP+ target cells and the CD19+/GFP-target cells was observed. This result demonstrated that the presence of the GFP antigen in the heterogeneous population primed the engineered T cells for CD19-specific killing generally and that cell killing by the engineered T cells was not limited to those cells expressing the priming antigen.

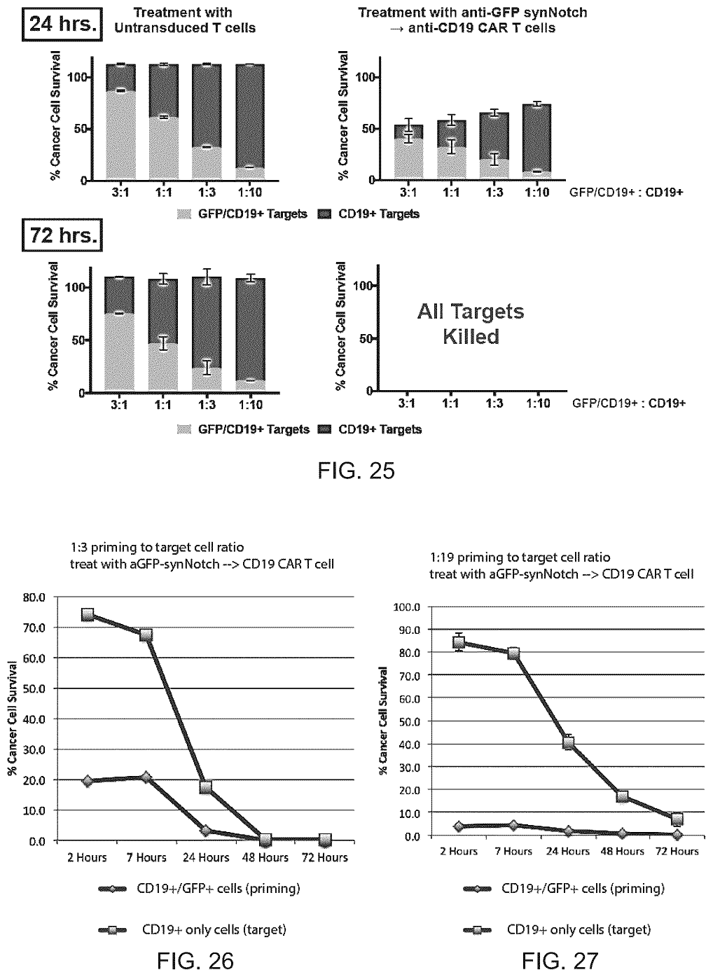

This effect of heterogeneous tumor cell killing was further quantified using populations with various ratios of GFP/CD19+ target cells to CD19+ only target cells. The heterogeneous population was treated with either anti-GFP synNotch→anti-CD19 CAR T cells or untransduced negative control T cells and time points were taken at 24 hours and 72 hours (FIG. 25). No cell killing was observed in any of the populations when treated with the untransduced cells. However, at 24 hours substantial cell killing was observed in all populations following treatment with the anti-GFP synNotch→anti-CD19 CAR T cells and by 72 hours all target cells were eliminated.

These results demonstrate that, regardless of the degree of tumor heterogeneity or the relative abundance/scarcity of the priming antigen, specific and effective tumor cells killing of heterogeneous tumors is achieved using such a priming→killing synNotch gated system. Put another way, the described system is effective and specific whether the heterogeneous tumor contains a high or a low percentage of priming antigen expressing cells.

Higher temporal resolution of the killing effects of the system on the priming and non-priming (“target”) populations in a 1:3 priming to target heterogeneous mix is provided in FIG. 26. As described above, all cells of both populations are eliminated by 72 hours post treatment.

As can be seen in FIG. 27, even when the ratio of priming cells to non-priming target cells is as low as 1:19, the CD19+ non-priming antigen target cells are effectively killed with similar temporal dynamics to that seen in FIG. 26. By 72 hours less than 10% of the original population of target cells remained. Collectively, these results demonstrate that synNotch gated tumor cell killing systems are effective at specifically treating heterologous tumors regardless of the relative representation of cells expressing the synNotch priming antigen within the tumor.

### Example 6: Controlled Expression of Immuno Suppressive Agents

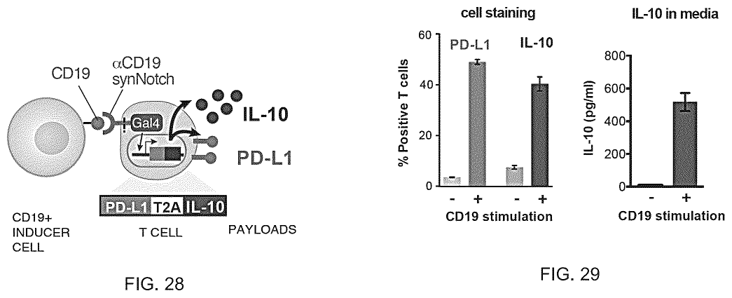

In addition to driving immune responses in cancer immunotherapy, the proteolytically cleavable chimeric polypeptides were also investigated for useful functions outside of cancer therapy. One application that was investigated using an exemplary synNotch was immunosuppression, e.g., in an an autoimmune setting. SynNotch T cells were engineered that drive the simultaneous production of paracrine inhibitory signals such as the cytokine, IL-10, and the T cell inhibitory ligand, PD-L1, which drives cell-to-contact inhibition (FIG. 28).

As assayed using CD19 stimulation, the anti-CD19 synNotch receptor was able to drive both suppressive agents (PD-L1 and IL-10) in response to stimulation with CD19+ K562s (FIG. 29). This example, as well as other examples of synNotch receptor T cells controlling non-native synthetic T cell responses, highlight the widely diverse applications of synNotch engineered cells to produce a spectrum of therapeutic agents that can enhance the effectiveness of the therapeutic cells or reprogram a tissue or disease environment to restore homeostasis and natural function.

### Example 7: Dual-Antigen Gated Cytokine Secretion Using Wild-Type Affinity and Enhanced Affinity TCRs

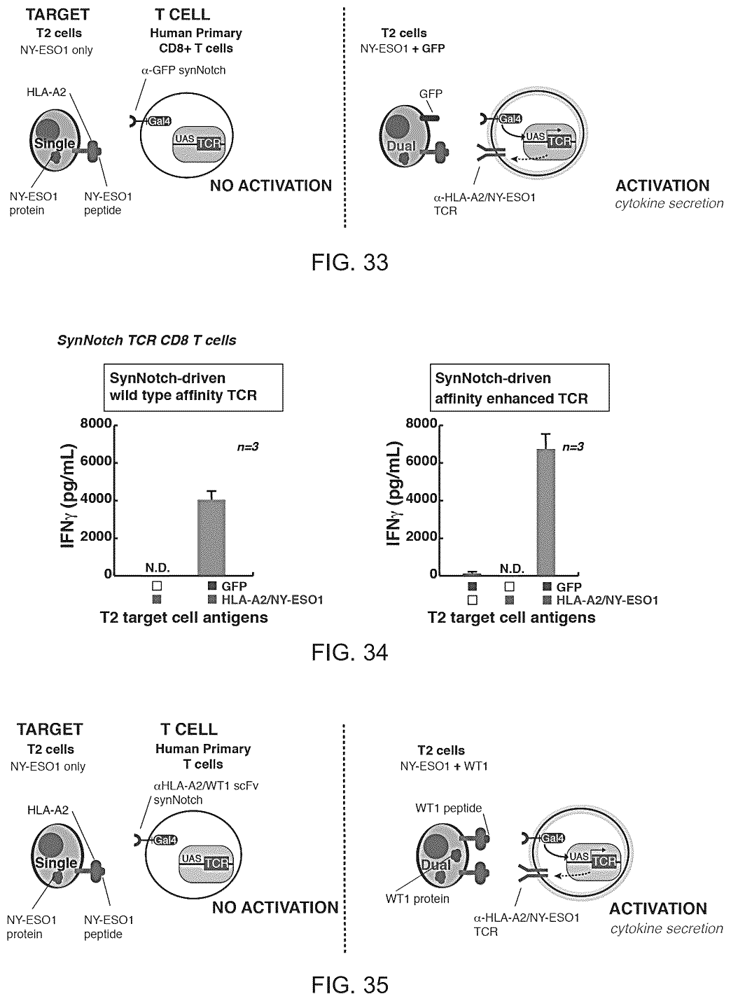

An additional dual-antigen AND-gate was designed for surface antigen gated TCR expression to test CD8 T cell cytokine secretion in response to targeting cells expressing both the target surface antigen (surface-expressed GFP) and an intracellular target antigen (NY-ESO). Specifically, as schematically presented in FIG. 33, CD8 T cells were engineered with an anti-GFP synNotch controlling the expression of an anti-HLA-A2/NY-ESO1 TCR such that release the cytokine interferon gamma occurs in response to T2 target cells expressing both surface-expressed-GFP and HLA-A2/NY-ESO1. The system was tested with both wild-type affinity and affinity enhanced anti-HLA-A2/NY-ESO1 TCR.

As shown in FIG. 34, CD8 T cells having anti-GFP synNotch gated expression of either TCR were activated and secreted IFNγ only in the presence of dual-antigen GFP+/HLA-A2/NY-ESO1+ T2 target cells. Furthermore, the level of CD8 T cell activation (as measured by IFNγ release) was dependent on the affinity of the TCR for its target antigen, as the affinity enhanced TCR cells produced nearly twice as much IFNγ, as compared to the wild-type affinity TCR cells, upon target cell engagement.

These results demonstrate dual-antigen (surface antigen and intracellular antigen) gated cytokine secretion using wild-type affinity and enhanced affinity TCRs. The data also show that the level of T cell activation and cytokine secretion may be modulated through the gated expression of TCRs having different affinities to their target antigen.

### Example 8: Dual-Antigen Gated CD4 T Cell Cytokine Secretion

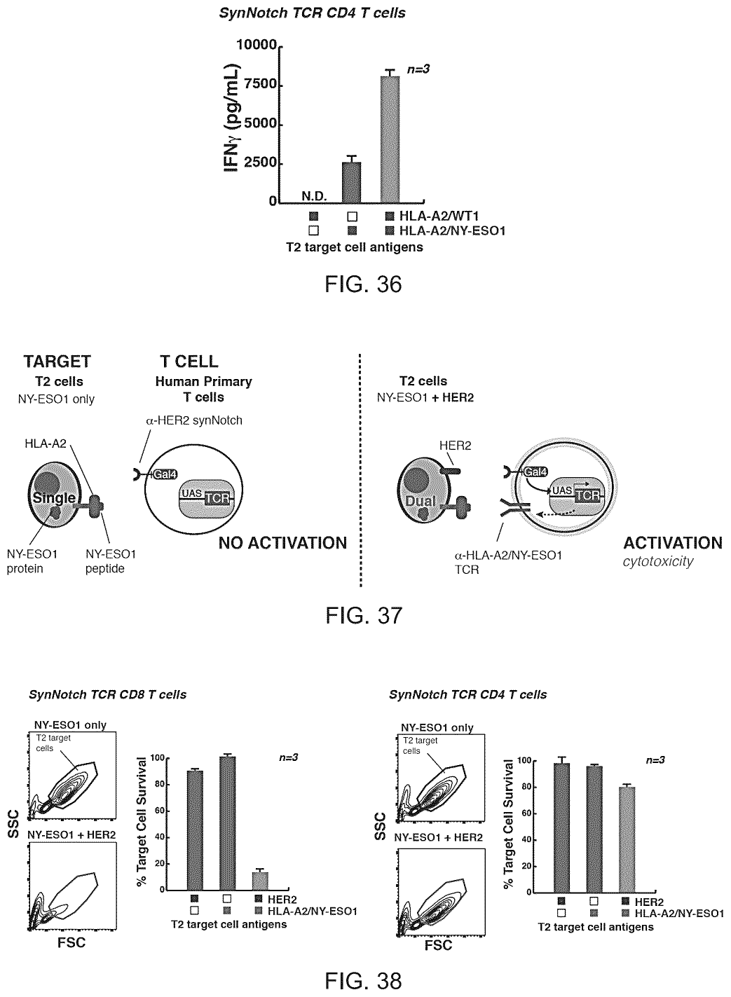

CD4 T cell activation and cytokine secretion was tested in CD4 T cells engineered with an anti-HLA-A2/WT1 synNotch controlling an anti-HLA-A2/NY-ESO1 TCR (as schematized in FIG. 35). As shown by the data provided in FIG. 36, the anti-HLA-A2/WT1 synNotch and anti-HLA-A2/NY-ESO1 TCR expressing cells release high levels of IFNγ specifically in response to T2 target cells expressing both HLA-A2/WT1 and HLA-A2/NY-ESO1 antigens. Some low level of cytokine secretion was seen in response to T2 target cells expressing only HLA-A2/NY-ESO1 due to partial recognition of HLA-A2 alone by the anti-HLA-A2/WT1 synNotch. Testing of multiple different anti-HLA-A2/WT1 scFvs in synNotch receptors has revealed low level measureable recognition of anti-HLA-A2 alone regardless of the specific WT1 peptide targeted. However, the high level increase of cellular activation and cytokine release in response to target cells expressing both antigens indicates that such background HLA-A2 partial recognition does not appreciably impact dual-antigen targeting and specificity.

### Example 9: Clinically Relevant Dual-Antigen Targeting

To further demonstrate the versatility of the described surface antigen/pMHC antigen AND-gating strategy using a clinically relevant antigen pair, both CD8 and CD4 T cells were engineered with an anti-HER2 synNotch controlling an anti-HLA-A2/NY-ESO1 TCR. Her2 and HLA-A2/NY-ESO1 are found co-expressed in various cancers, including breast cancer and glioma. A schematic depicting this gating/targeting strategy is provided in FIG. 37.

As shown in the results provided in FIG. 38, both the engineered CD8 and CD4 T cells specifically killed target T2 cells expressing both target antigens (HER2 and HLA-A2/NY-ESO1). Control cells expressing only one of the two antigens were not affected. Notably, the specific killing of dual-antigen target cells by the engineered CD4 cells demonstrated that surface antigen/pMHC antigen recognition circuits can induce target killing in a CD8-independent manner.

Collectively, the results of this example demonstrate that cells expressing clinically relevant antigen pairs, including those targeting at least one intracellular antigen, can be effectively targeted and killed using engineered dual-antigen gated CD8 T cells and/or dual-antigen gated CD4 T cells.

### Example 10: Dual-Antigen Targeting with pMHC-Sensing synNotch Controlling CAR Expression

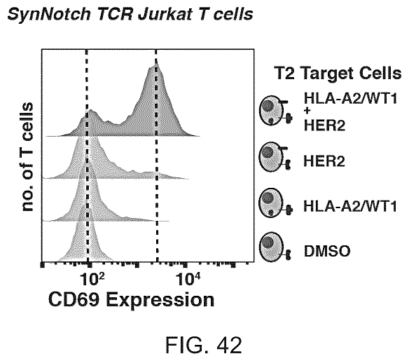

To further demonstrate the versatility of this approach, additional dual-antigen AND-gates containing a CAR were designed and tested targeting cells expressing both a surface target antigen and an intracellular target antigen. An exemplary system is depicted in FIG. 41, where, as designed and engineered into T cells, expression of an anti-Her2 CAR is induced by the freed intracellular domain of an anti-HLA-A2/WT1 synNotch. This circuit serves as an example of a pMHC-specific synNotch receptor controlling expression of a surface antigen-specific CAR. When presented with a T2 target cell expressing Her2 only, expression of the anti-Her2 CAR is not induced and no T cell activation occurs. However, when presented with a T2 target cell expressing both WT1 and Her2, binding of surface-HLA-A2/WT1 to the anti-HLA-A2/WT1 synNotch induces cleavage of the Notch polypeptide, releasing a GAL4 transcription activator which binds a UAS driving expression of the anti-Her2 CAR. Binding of the expressed anti-Her2 CAR to Her2 on the target cell causes activation of the T cell. The resulting activation, as measured using CD69 expression, of a Jurkat T cell line engineered to express this dual-antigen circuit is provided in FIG. 42. The results of this assay showed that T cell activation (i.e. CD69 upregulation) was dependent on expression of both target antigens by the target cells, again demonstrating the robust specificity of dual-antigen synNotch AND-gates. This example, with a pMHC-specific synNotch receptor controlling expression of a surface antigen-specific CAR, also shows the wide diversity of antigen combinations that may be employed in dual-antigen synNotch AND-gates.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A chimeric polypeptide comprising, from N- terminal to C- terminal and in covalent linkage:
a) an extracellular domain comprising a specific binding member that specifically binds to a peptide-major histocompatibility complex (peptide-MHC);
b) a proteolytically cleavable Notch receptor polypeptide comprising one or more proteolytic cleavage sites; and
c) an intracellular domain comprising a transcriptional activator, wherein binding of the specific binding member to the peptide-MHC induces cleavage of the Notch receptor polypeptide at the one or more proteolytic cleavage sites, thereby releasing the intracellular domain.

2. The chimeric polypeptide of claim 1, wherein the specific binding member comprises an antibody.

3. The chimeric polypeptide of claim 2, wherein the antibody is a nanobody, a diabody, a triabody, or a minibody, a F(ab′)₂ fragment, a Fab fragment, a single chain variable fragment (scFv) or a single domain antibody (sdAb).

4. The chimeric polypeptide of claim 1, wherein the specific binding member specifically binds a peptide-MHC comprising an intracellular cancer antigen peptide.

5. The chimeric polypeptide of claim 4, wherein the intracellular cancer antigen peptide is a WT1 peptide or a NY-ESO peptide.

6-72. (canceled)

73. A host cell comprising:
a) a nucleic acid encoding a chimeric polypeptide comprising, from N-terminal to C-terminal and in covalent linkage:
i) an extracellular domain comprising a specific binding member that specifically binds to a target molecule present on the surface of a cancer cell;
ii) a proteolytically cleavable Notch receptor polypeptide comprising one or more proteolytic cleavage sites; and
iii) an intracellular domain comprising a transcriptional activator;
b) a nucleic acid encoding an immune suppression factor operably linked to a transcriptional control element responsive to the transcriptional activator, wherein binding of the specific binding member to the target molecule induces cleavage of the Notch receptor polypeptide at the one or more proteolytic cleavage sites, thereby releasing the intracellular domain, activating the transcriptional control element and causing the immune suppression factor to be expressed.

74. The method of claim 73, wherein the target molecule is a tissue specific molecule.

75. The method of claim 73, wherein the target molecule is an organ specific molecule.

76. The method of claim 73, wherein the target molecule is a cell type specific molecule.

77. The method of claim 73, wherein the target molecule is an autoantigen.

78. The method of claim 73, wherein the immune suppression factor is an immunosuppressive cytokine.

79. The method of claim 78, wherein the immunosuppressive cytokine is IL-10.

80. The method of claim 73, wherein the immune suppression factor is a cell-to-cell signaling immunosuppressive ligand.

81-83. (canceled)

84. A method of killing a heterogeneous tumor, the method comprising:
contacting a heterogeneous tumor comprising a first cell expressing a killing antigen and a second cell expressing the killing antigen and a priming antigen with an engineered immune cell comprising:
a proteolytically cleavable chimeric polypeptide that specifically binds the priming antigen and contains a Notch regulatory region;
a nucleic acid sequence encoding a therapeutic polypeptide that specifically binds the killing antigen; and
a transcriptional control element operably linked to the nucleic acid that is responsive to the proteolytically cleavable chimeric polypeptide,
wherein binding of the proteolytically cleavable chimeric polypeptide to the priming antigen activates the transcriptional control element to induce expression of the therapeutic polypeptide which, when bound to the killing antigen, kills the first and second cells of the heterogeneous tumor.

85. The method of claim 84, wherein the therapeutic polypeptide is a chimeric antigen receptor (CAR).

86. The method of claim 84, wherein the therapeutic polypeptide is a T cell Receptor (TCR).

87. The method of claim 84, wherein the therapeutic polypeptide is a therapeutic antibody.

88. The method of claim 84, wherein the therapeutic polypeptide is a chimeric bispecific binding member.

89. The method of claim 84, wherein at least one of the priming antigen or the killing antigen is an intracellular antigen presented in the context of MHC.

90. The method of claim 84, wherein both the priming antigen and the killing antigen are intracellular antigens presented in the context of MHC.
